(19) United States
(12) Patent Application Publication
(10) Pub. No.: US 2020/0071326 A1
(43) Pub. Date: Mar. 5, 2020

(54) TAM KINASE INHIBITORS

(71) Applicant: Syros Pharmaceuticals, Inc., Cambridge, MA (US)

(72) Inventors: Yi Zhang, Belmont, MA (US); Kathryn Austgen, Hingham, MA (US); Claudio Chuaqui, Arlington, MA (US); Goran Malojcic, Boston, MA (US); William Sinko, Roslindale, MA (US); Huiping Amy Guan, Acton, MA (US); Tracey Lodie Savoie, Northbridge, MA (US)

(21) Appl. No.: 16/604,658

(22) PCT Filed: Apr. 13, 2018
(86) PCT No.: PCT/US2018/027437

§ 371 Date: Oct. 11, 2019

Related U.S. Application Data
(60) Provisional application No. 62/485,631, filed on Apr. 14, 2017.

(51) Int. Cl. C07D 471/04

(52) U.S. Cl. CPC C07D 471/04

(57) ABSTRACT

Described herein are compounds, methods of making such compounds, compositions (e.g., pharmaceutical compositions/medicaments) that include such compounds, and methods of using such compounds to treat diseases, such as cancer.

### CROSS-REFERENCE TO RELATED APPLICATIONS

This application is a U.S. National Phase Application under 35 U.S.C. § 371 of International Application No. PCT/US2018/027437, filed Apr. 13, 2018, which claims priority to and the benefit of the priority date of U.S. provisional application No. 62/485,631, filed Apr. 14, 2017, the entire content of each of which is hereby incorporated by reference herein.

### BACKGROUND

The TAM receptor tyrosine kinases (TYRO3, AXL and MERTK; the “TAM kinases”) constitute a family of receptor tyrosine kinases (RTKs) that play several important roles in normal macrophage physiology, including regulation of cytokine secretion and clearance of apoptotic cells. Modulation of TAM kinases has been shown to be useful in the treatment of a variety of diseases.

### SUMMARY

The present disclosure provides compounds represented by structural Formula I:

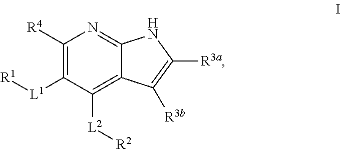

a prodrug thereof, or a pharmaceutically acceptable salt thereof, wherein:

L¹ is a bond, C₁-C₃ alkylene, —CH═CH—*, —C≡C—*, —N(R⁵)—C(O)—*, —N(R⁵)—C(O)—CH₂*, —C(O)—N(R⁵)—*, —C(O)—N(R⁵)—CH₂^{-*}, —O—(C₀—C₂ alkylene)-*, —N(R⁵)—S(O)₂—*, —N(R⁵)—S(O)₂—CH₂—*, —S(O)₂—N(R⁵)—*, —S(O)₂—N(R⁵)—CH₂—*, —N(R⁵)—(C₀-C₂ alkylene)—*, —O—C(O)—*, —O—C(O)—CH₂—*, —C(O)—O—*, or —C(O)—O—CH₂—*, wherein “*” represents a portion of L¹ bound to R¹; and R⁵ is hydrogen, C₁-C₄ alkyl, or C₃-C₇ cycloalkyl, and any alkylene, alkyl or cycloalkyl portion of L¹, if present, is optionally substituted;

R¹ is halogen, C₁-C₃ alkyl, aryl, heteroaryl, heterocyclyl or carbocyclyl, wherein R¹ is optionally substituted with up to four different substituents;

L² is —O—(C₀-C₃ alkylene)-† or —N(R⁶)—(C₀-C₃ alkylene)-†, wherein “†” represents a portion of L² bound to R², R⁶ is hydrogen, C₁-C₄ alkyl, or C₃-C₇ cycloalkyl, and any alkylene, alkyl or cycloalkyl portion of L², if present, is optionally substituted;

R² is C₁-C₃ alkyl, aryl, heteroaryl, heterocyclyl or carbocyclyl, wherein R² is optionally substituted with up to four different substituents;

R^{3a} is —C₁-C₆ alkyl, —C₂-C₆ alkenyl, —C₂-C₆ alkynyl, —(C₁-C₆ alkylene)—O—(C₁-C₆ alkyl), —(C₀-C₃ alkylene)-aryl, —(C₀-C₃ alkylene)-carbocyclyl, —(C₀-C₃ alkylene)-heterocyclyl, or —(C₀-C₃ alkylene)-heteroaryl, wherein R^{3a} is optionally substituted;

R^{3b} is hydrogen, halogen, or optionally substituted —C₁-C₄ alkyl; and

R⁴ is hydrogen, halogen, —C₁-C₄ alkyl, or —O—(C₁-C₄ alkyl), wherein any alkyl portion of R⁴ is optionally substituted.

In some embodiments of Formula I, when L₁ is a bond, C₁ alkylene, —NH—, —C(O)—O—*, or —O—, then R¹ is other than C₁ alkyl or C₁ alkyl substituted with halogen. In some embodiments of Formula I, when L₁ is a bond, then R¹ is other than cyclopropyl. Thus, and for example, a compound disclosed herein can be represented by structural formula I, with the proviso that when L₁ is a bond, then R¹ is other than cyclopropyl, and when L¹ is C₁ alkylene, —NH—, —C(O)—O—*, or —O—, then R¹ is other than C₁ alkyl or C₁ alkyl substituted with halogen.

A provided compound and/or a pharmaceutical composition containing the compound may be used to inhibit one or more TAM kinases, at least at a site of interest (e.g., in a tissue, cell, or subcellular location in vivo or in vitro). The provided compound or pharmaceutical composition may be used to inhibit cell proliferation and/or to inhibit an activity of one or more TAM kinases (i.e., MERTK, AXL and/or TYRO3) and may have increased specificity for the TAM kinase(s) relative to FLT3 (e.g., a provided compound or composition may inhibit one or more TAM kinases without inhibiting FLT3 or while inhibiting FLT3 to a significantly lesser extent).

In some embodiments, a provided compound or pharmaceutical composition is contacted with and/or administered to cells, such as cancer cells, which may be or comprise cells of a breast cancer, ovarian cancer, glioblastoma, pancreatic ductal adenocarcinoma, non-small cell lung cancer (NSCLC), colorectal cancer, leukemia, lymphoma, gastric cancer, prostate cancer, pituitary adenoma, melanoma or rhabdomyosarcoma. A provided compound and/or pharmaceutical composition can be administered to a cancer patient who is resistant to a checkpoint inhibitor. Checkpoint inhibitors include, but are not limited to, PD-1 inhibitors (e.g., avelumab, nivolumab, and pembrolizumab), PD-L1 inhibitors (e.g., atezolizumab and durvalumab), and CTLA4 inhibitors (e.g., ipilimumab). In some embodiments, a provided compound or pharmaceutical composition is administered to a patient who has a cancer associated with elevated myeloid infiltration.

### DETAILED DESCRIPTION

Compounds of this invention include those described generally above, and those further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito, 1999, and “March's Advanced Organic Chemistry,” ^{5th} Ed., Eds. M. B. Smith and J. March, John Wiley & Sons, New York, 2001, the entire contents of which are hereby incorporated by reference.

Compounds described herein may be biologically and/or therapeutically active. Further, in some embodiments, one or more compounds described herein can be provided and/or utilized in any of a variety of forms such as, for example, salt forms, protected forms, isomeric forms (e.g., optical and/or structural isomers), and isotopic forms. One of ordinary skill in the art will appreciate that certain compounds have structures that can exist in one or more stereoisomeric forms. In some embodiments, such compounds may be utilized in accordance with the present disclosure in the form of an individual enantiomer, diastereomer or geometric isomer, or may be in the form of a mixture of stereoisomers; in some embodiments, such compounds may be utilized in accordance with the present disclosure in a racemic mixture form. One of ordinary skill in the art will appreciate that certain compounds have structures that can exist in one or more tautomeric forms and that certain compounds have structures that permit isotopic substitution (e.g. ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ³⁵C; ¹⁸F for ¹⁹F; ¹³¹I for ¹²⁷I; etc.). In some embodiments, such compounds may be utilized as described herein in one or more isotopically modified forms or mixtures thereof, and reference to a particular compound may relate to a specific form of that compound. Where a compound exists or is found in nature, that compound may be provided and/or utilized in accordance with the present invention in a form different from that in which it exists or is found in nature. Similarly, a composition described herein (e.g., a pharmaceutical composition) can be non-naturally occurring. A compound preparation including a different level, amount, or ratio of one or more individual forms than a reference preparation or source (e.g., a natural source) of the compound may be considered a different form of the compound as described herein. Thus, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may also be considered to be a different form.

The terms “aliphatic” or “aliphatic group,” as used herein, mean a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclyl,” “cycloaliphatic” or “cycloalkyl”), that has a single point of attachment to the rest of the molecule. Unless otherwise specified, aliphatic groups contain 1-6 aliphatic carbon atoms (“C₁-C₆”). In various embodiments, aliphatic groups can contain 1-5 aliphatic carbon atoms (“C₁-C₅”), 1-4 aliphatic carbon atoms (“C₁-C₄”), 1-3 aliphatic carbon atoms (“C₁-C₃”), or 1-2 aliphatic carbon atoms (“C₁-C₂”). In some embodiments, “cycloaliphatic” (or “carbocycle” or “cycloalkyl”) refers to a monocyclic hydrocarbon containing 3-6 aliphatic carbon atoms (“C₃-C₆”) that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl and (cycloalkyl)alkenyl.

The term “alkyl,” as used herein, means an unbranched or branched chain, saturated, monovalent hydrocarbon residue containing 1 to 10 carbon atoms. Suitable alkyl groups include methyl, ethyl, n- and iso-propyl, n-, sec-, iso- and tert-butyl, neopentyl, and the like.

The term “alkenyl,” as used herein, means monovalent straight or branched chain groups of, unless otherwise specified, from 2 to 10 carbon atoms (“C₂-C₁₀”) containing one or more carbon-carbon double bonds and is exemplified by ethenyl, propenyl, butenyl, pentenyl, hexenyl, and the like.

The term “alkynyl,” as used herein, means monovalent straight or branched chain groups from 2 to 10 carbon atoms (“C₂-C₁₀”) containing at least one carbon-carbon triple bond. Suitable alkynyl groups include ethynyl, propynyl, butynyl, pentynyl, hexynyl, and the like.

The term “heteroatom,” as used herein, means one or more of oxygen, sulfur, nitrogen, phosphorus, or silicon (including any oxidized form of nitrogen, sulfur, phosphorus, or silicon; and the quaternized form of any basic nitrogen or a substitutable nitrogen of a heterocyclic ring, for example N (as in 3,4-dihydro-2H-pyrrolyl-

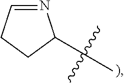 [SMILES: *C1CCC=N1]

NH (as in pyrrolidinyl-

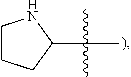 [SMILES: *C1CCCN1]

NR\textcircled{ } (as in N-substituted 2-pyrrolidinyl-

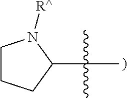

or ⁺NR\textcircled{ } (as in N-substituted 1-pyrrolidinyl-

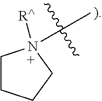

The term “heteroalkyl,” as used herein, refers to an alkyl group, wherein one or more carbon atoms is replaced with a heteroatom selected from oxygen, sulfur, or nitrogen.

The term “unsaturated,” as used herein, means that a moiety has one or more units of unsaturation. A unit of unsaturation can be a carbon-carbon double bond (i.e., —C═C—) or a carbon-carbon triple bond (i.e., —C≡C).

The term “bivalent C₂₋₈ (or C₂₋₆) unsaturated, straight or branched, hydrocarbon chain,” as used herein, means bivalent alkenylene and alkynylene chains that are straight or branched as defined herein and have one or more units of unsaturation.

The term “alkylene,” as used herein, means a straight or branched bivalent alkyl group. Exemplary alkylenes include —CH₂—, —CH₂CH₂—, —CH(CH₃)—, —CH₂CH(CH₃)—, —CH(CH₃)CH₂—, etc. In some embodiments, an “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a bivalent alkyl group in which one or more hydrogen atoms are replaced with a substituent. Suitable substituents include those described below for a substituted aliphatic group.

The term “alkenylene,” as used herein, means a bivalent alkenyl group. A substituted alkenylene chain is a bivalent alkenyl group containing at least one double bond in which one or more hydrogen atoms are optionally replaced with a substituent. Suitable substituents include those described below for a substituted aliphatic group.

The term “halogen,” as used herein, means F, Cl, Br, or I.

The term “aryl,” as used herein, means monocyclic, bicyclic and tricyclic ring systems having a total of six to fourteen ring atoms, wherein each ring atom is carbon and at least one ring in the system is aromatic and wherein each ring in the system contains three to seven ring members. The term “aryl” may be used interchangeably with the term “aryl ring.” In certain embodiments, “aryl” refers to an aromatic ring system that includes, but is not limited to, phenyl, biphenyl, naphthyl, and anthracyl, which may bear one or more substituents. Also encompassed by the term “aryl,” as it is used herein, is a group in which an aromatic ring is fused to one or more non-aromatic rings, such as indanyl, naphthimidyl, or tetrahydronaphthyl.

The terms “heteroaryl” and “heteroar-,” used alone or as part of a larger moiety (e.g., “heteroaralkyl” or “heteroaralkoxy”), refer to groups having 5 to 14 ring atoms, preferably 5, 6, or 9 ring atoms; having 6, 10, or 14 7C electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. When used in reference to a ring atom of a heteroaryl, the term “nitrogen” includes a substituted nitrogen. As an example, in a heteroaryl ring having 0-3 heteroatoms selected from oxygen, sulfur or nitrogen, nitrogen may be N (as in pyridinyl-

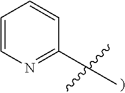 [SMILES: *c1ccccn1]

or ⁺NR\textcircled{ } (as in N-substituted pyridinyl-

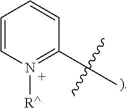

Heteroaryl groups may be mono-, bi- or tricyclic and include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, and pteridinyl. The terms “heteroaryl” and “heteroar-”, as used herein, also include groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, 5,6,7,8-tetrahydroquinolinyl, 5,6,7,8-tetrahydroisoquinolinyl, and pyrido[2,3-b]-1,4-oxazin-3(4H)-one. When a heteroaryl ring is fused to an aryl ring, the term “heteroaro” is used to refer to the heteroaryl ring that is fused to the aryl ring. The term “heteroaryl” may be used interchangeably with the terms “heteroaryl ring”, “heteroaryl group”, or “heteroaromatic”, any of which are optionally substituted.

As used herein, the terms “heterocycle,” “heterocyclyl,” “heterocyclic radical,” and “heterocyclic ring” are used interchangeably and refer to a stable 4- to 7-membered monocyclic, 7-11-membered bicyclic or 10-16-membered tricyclic heterocyclic moiety that is either saturated or partially unsaturated, and having, in addition to carbon atoms, one or more, preferably one to four, heteroatoms, as defined above. When used to refer to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl-

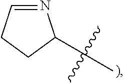 [SMILES: *C1CCC=N1]

NH (as in pyrrolidinyl-

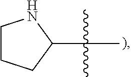 [SMILES: *C1CCCN1]

NR\textcircled{ } (as in N-substituted 2-pyrrolidinyl-

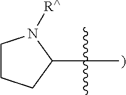

or ⁺NR\textcircled{ } (as in N-substituted 1-pyrrolidinyl-

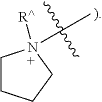

A heterocyclic ring can be attached to its pendant group at any heteroatom or carbon atom that results in a stable structure and any of the ring atoms can be optionally substituted. A heterocyclyl group may be mono-, bi- or tricyclic. Examples of such saturated or partially unsaturated heterocyclic radicals include, without limitation, tetrahydrofuranyl, tetrahydrothiophenyl, pyrrolidinyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heterocyclyl, or cycloaliphatic rings, such as indolinyl, 3H-indolyl, chromanyl, 1,2,3,4-tetrahydroisoquinolinyl or 1,2,3,4-tetrahydroquinolinyl. For purposes of clarity, a “heterocyclic” ring system includes a saturated or partially unsaturated, but not aromatic, ring having one or more heteroatoms, wherein the ring is either monocyclic or fused to one or more aryl heterocyclyl or cycloaliphatic rings. When a heterocyclic ring is fused to an aryl ring, the term “heterocyclo” is used to refer to the heterocyclic ring that is fused to the aryl ring. A “saturated heterocyclic ring” refers to a saturated ring having one or more heteroatoms, wherein the ring is monocyclic or fused to one or more saturated cycloaliphatic rings.

As used herein, the term “partially unsaturated” refers to a ring moiety that includes at least one double or triple bond. The term “partially unsaturated” is intended to encompass rings having multiple sites of unsaturation, but it is not intended to include aryl or heteroaryl moieties, as herein defined.

Compounds may contain one or more “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be independently deuterium, halogen; —(CH₂)_{0.4}R^{o}; —(CH₂)₀₋₄OR^{o}; —O(CH₂)₀₋₄R^{o}; —O—(CH₂)₀₋₄C(O)OR^{o};—(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o}C(O)R^{o}; —N(R^{o}C(S)R^{o}; (CH₂)₀₋₄N(R^{o}C(O)NR^{o}₂; —N(R^{o}C(S)NR^{o}₂; —(CH₂)₀₋₄N(R^{o}C(O)OR^{o}; —N(R^{o}N(R^{o}C(O)R^{o}; —N(R^{o}N(R^{o}C(O)NR^{o}₂; —N(R^{o}N(R^{o}C(O)OR^{o}; —(CH₂)₀₋₄C(O)R^{o}; —C(S)R^{o}; —(CH₂)₀₋₄C(O)OR^{o}; —(CH₂)₀₋₄—C(O)—N(R^{o})—S(O)₂—R^{o}; —C(NCN)NR^{o}₂; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}; SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; C(O)CH₂C(O)R^{o}; —C(NOR^{o}R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o}S(O)₂NR^{o}₂; —N(R^{o}S(O)₂R^{o}; —N(OR^{o}R^{o}; —C(NOR^{o}NR^{o}₂; —C(NH)NR^{o}₂; —P(O)₂R^{o}; —P(O)R^{o}₂; —P(O)(OR^{o}₂; —OP(O)R^{o}₂; —OP(O)(OR^{o}₂; —OP(O)(OR^{o}R^{o}, —SiR^{o}₃; —(C₁₋₄ straight or branched)alkylene)O—N(R^{o}₂; or —(C₁₋₄ straight or branched)alkylene)C(O)O—N(R^{o}₂, wherein each R^{o}may be substituted as defined below and is independently hydrogen, C₁₋₆ aliphatic, —CH₂Ph, —O(CH₂)₀₋₁Ph, —CH₂—(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o}(or the ring formed by taking two independent occurrences of R^{o}together with their intervening atoms), may be, independently, halogen, —(CH₂)₋₂R^{⋅}, —(halole), —(CH₂)₀₋₂OH, —(CH₂)₀₋₂OR^{⋅}, —(CH₂)₀₋₂CH(OR^{⋅})₂; —O(halole^{⋅}), —CN, —N₃, —(CH₂)₀₋₂C(O)R^{⋅}, —(CH₂)₀₋₂C(O)OH, —(CH₂)₀₋₂C(O)OR^{⋅}, —(CH₂)₀₋₂SR^{⋅}, —(CH₂)₀₋₂SR^{⋅}, —(CH₂)_{0- 2}NH₂, —(CH₂)₀₋₂NHR^{⋅}, —(CH₂)₀₋₂NR^{⋅}₂, —NO₂, —SiR^{⋅}₃, —OSiR^{⋅}₃, —C(O)SR^{⋅}, —(C₁₋₄ straight or branched alkylene)C(O)OR^{⋅}, or —SSR^{⋅} wherein each R^{⋅} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o}include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{⋅}, —(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{⋅}, —(haloR^{⋅}), —OH, —OR^{†}, —O(haloR^{†}), —CN, —C(O)OH, —C(O)OR^{†}, —NH₂, —NUR^{†}, —NR^{†}₂, or —NO₂, wherein each R^{†} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

Structures/compounds depicted or described herein may include all isomeric (e.g., enantiomeric, diastereomeric, and geometric (or conformational)) forms of the depicted structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein may encompass compounds that differ from the depicted structure(s) only in the presence of one or more isotopically enriched atoms. For example, compounds having the presented structures including the replacement of hydrogen by deuterium or tritium, or the replacement of a carbon by a ¹³C- or ¹⁴C-enriched carbon are within the scope of this invention. Such compounds may be useful, for example, as analytical tools, as probes in biological assays, or as therapeutic agents in accordance with the present invention. In some embodiments, the R¹ group of formula I comprises one or more deuterium atoms.

As used herein, “ [symbol] ” appearing on a structure and joining a functional group to the structure in the position of a bond generally indicates a mixture of, or either of, the possible isomers, e.g., containing (R)-and (S)-stereochemistry.

As used herein, “ [symbol] ” appearing across or at the end of a bond indicates a point of attachment between two atoms. For example:

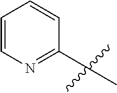 [SMILES: *c1ccccn1]

means that the pyridine ring above is bound through the indicated ring carbon atom to an undepicted structure on which it is a substituent.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, the term “pharmaceutical composition” refers to a composition in which an active agent (e.g., a compound described herein or a prodrug thereof) is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. A pharmaceutical composition may be specially formulated for administration in solid or liquid form, including those adapted for: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. A compound or pharmaceutical composition described herein can also be referred to as a “medicament.”

As used herein, the term “pharmaceutically acceptable” characterizes an agent (e.g., a carrier used to formulate a pharmaceutical composition described herein) as one that is compatible with the other ingredients of the composition and not deleterious to the recipient thereof (e.g., non-toxic in the amount provided).

Other Definitions: As used herein, the term “administration” typically refers to the administration of a compound or pharmaceutical composition to a subject/patient or system. One of ordinary skill in the art will be aware of a variety of routes that may, in appropriate circumstances, be utilized for administration to a subject (e.g., a human). For example, administration may be ocular, oral, topical, etc. In some embodiments, administration may be bronchial (e.g., by bronchial instillation), buccal, dermal (which may be or comprise, for example, one or more of topical application to the dermis, intradermal, interdermal, transdermal, etc.), enteral, intra-arterial, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time). Administration may involve continuous dosing (e.g., perfusion) for at least a selected period of time. In some embodiments, administration of a particular compound may be achieved by administration of a composition to a system or by administration of a pharmaceutical composition to a subject, the composition including or otherwise delivering the compound to the system or subject (or to a relevant part thereof or site therein). Thus, administration of a compound to a subject may be achieved by administration of a pharmaceutical composition comprising the compound, a prodrug thereof, or a pharmaceutically acceptable salt thereof. Thus, administration of a compound may be achieved by administration of the compound per se or by administration of a prodrug or any other variant of the compound that is metabolized to the compound upon administration of the composition. It is to be understood that where a compound of the invention is useful, a prodrug that provides that compound is also useful. Accordingly, the treatments and methods of use described herein can be carried out with a compound described herein or a prodrug thereof.

Two events or entities are “associated” with one another, as that term is used herein, if the presence, level and/or form of one is correlated with that of the other. For example, a particular entity (e.g., polypeptide (e.g., a TAM kinase), genetic signature, metabolite, microbe, event (e.g., myeloid infiltration), etc.) is considered to be associated with a particular disease (e.g., cancer) or disorder if its presence, level and/or form correlates with incidence of and/or susceptibility to the disease or disorder (e.g., across a relevant population). Two or more entities can be physically “associated” with one another if they interact, directly or indirectly, so that they are and/or remain in physical proximity with one another. Two or more entities that are physically associated with one another can be covalently linked to one another or non-covalently associated, for example by means of hydrogen bonds, van der Waals interaction, hydrophobic interactions, magnetism, or combinations thereof.

As used herein, the term “binding” typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts, including where interacting entities or moieties are studied in isolation or in the context of more complex systems (e.g., while covalently or otherwise associated with a carrier entity and/or in a biological system or cell (e.g., in culture)).

As used herein, the term “biologically active” refers to an observable biological effect or result achieved by an agent or entity of interest (e.g., a compound described herein). For example, a specific binding interaction can be a biological activity. In some embodiments, modulation (e.g., induction, enhancement, or inhibition) of a biological pathway or event is a biological activity. The presence or extent of a biological activity can be assessed through detection of a direct or indirect product produced by a biological pathway or event of interest.

As used herein, the term “biological sample” typically refers to a sample obtained or derived from a biological source (e.g., a tissue or organism (e.g., an animal or human patient) or cell culture) of interest. The biological sample can be or can comprise a biological tissue or fluid. For example, a biological sample may be or may comprise bone marrow; blood; blood cells; ascites; tissue or fine needle biopsy samples; cell-containing body fluids; free floating nucleic acids; sputum; saliva; urine; cerebrospinal fluid (CSF), peritoneal fluid; pleural fluid; feces; lymph; gynecological fluids; tissue swabbed from the skin or mucus membrane (e.g., in the nose, mouth, or vagina); washings or lavages such as a ductal lavages or broncheoalveolar lavages; aspirates; scrapings; tissue biopsy specimens; surgical specimens; or other body fluids, secretions, and/or excretions and/or cells therefrom. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means (e.g., by biopsy (e.g., fine needle aspiration or tissue biopsy), surgery, collection of body fluid (e.g., blood, lymph, feces etc.)). In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

As used herein, the term “biomarker” refers to an entity whose presence, level, or form, correlates with a particular biological event or state of interest, so that it is considered to be a “marker” of that event or state. For example, in some embodiments, a biomarker may be or comprises a marker for a particular disease (e.g., cancer or a particular type of cancer) or disease state, or for likelihood that a particular disease or disorder may develop. In some embodiments, a biomarker may be or comprise a marker for a particular disease or therapeutic outcome, or the likelihood thereof. Thus, a biomarker can be predictive, prognostic, or diagnostic of a biological event or state of interest. A biomarker may be an entity of any chemical class. For example, a biomarker can be or can comprise a nucleic acid, polypeptide, lipid, carbohydrate, small molecule, inorganic agent (e.g., a metal or ion), or a combination thereof. In some embodiments, a biomarker is a cell surface marker; in other embodiments, the biomarker is intracellular; in yet other embodiments, the biomarker is found outside of cells (e.g., it is secreted or is otherwise generated or present outside of cells, e.g., in a body fluid such as blood, plasma, urine, tears, saliva, CSF, etc.).

As used herein, the terms “cancer,” “malignancy,” “neoplasm,” “tumor,” and “carcinoma,” refer to cells that exhibit relatively abnormal, uncontrolled, and/or autonomous growth, so that they exhibit an aberrant growth phenotype characterized by a significant loss of control of cell proliferation. A tumor may consist of or comprise cells that are precancerous (e.g., benign), malignant, pre-metastatic, metastatic, and/or non-metastatic. The present disclosure specifically identifies certain cancers to which its teachings may be particularly relevant. A relevant cancer may be characterized by a solid tumor or by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

As used herein, “carrier” refers to a diluent, adjuvant, excipient, or vehicle with which a composition (e.g., a compound disclosed herein) is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. Carriers can include one or more solid components.

As used herein, the term “comparable” refers to two or more agents, entities, situations, sets of conditions, etc., that are not identical to one another but are sufficiently similar to permit comparison therebetween so that one of ordinary skill in the art will appreciate that conclusions may reasonably be drawn based on differences or similarities observed. In some embodiments, comparable sets of conditions, circumstances, individuals, or populations are characterized by a plurality of substantially identical features and one or a small number of varied features. One of ordinary skill in the art will understand, in context, what degree of identity is required in any given circumstance for two or more such agents, entities, situations, sets of conditions, etc. to be considered comparable. For example, one of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by, or indicative of the variation in, those features that are varied.

As used herein, the term “combination therapy” refers to situations in which a subject is simultaneously exposed to two or more therapeutic regimens (e.g., two or more therapeutic agents, including one or more compounds as described herein). The two or more regimens may be administered simultaneously or sequentially (e.g., all doses of a first regimen are administered prior to administration of any doses of a second regimen). In other embodiments, such compounds are administered in overlapping dosing regimens. “Administration” of a combination therapy may involve administration of one or more compounds to a subject receiving the other compound(s) in the combination. For clarity, combination therapy does not require that individual compounds be administered together in a single composition (or even necessarily at the same time or by the same route of administration), although in some embodiments, two or more compounds may be administered together in a combination composition, or even in a combination compound (e.g., as part of a single chemical complex or covalent entity).

As used herein, the terms “dosage form” or “unit dosage form” refer to a physically discrete unit of an active agent (e.g., a therapeutic or diagnostic agent (e.g., a compound described herein) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). One of ordinary skill in the art would appreciate that the total amount of an agent administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic agent (e.g., compound) has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. The doses within a dosing regimen may contain the same unit dose amount or may contain at least two different dose amounts. For example, a dosing regimen can comprise a first dose in a first dose amount, followed by one or more additional doses in a second dose amount that is the same as the first dose amount.

Alternatively, a dosing regimen can comprise a first dose in a first dose amount followed by one or more additional doses in a second dose amount that is different from the first dose amount. The dosing regimen can be correlated with a desired or beneficial outcome when administered across a relevant population (i.e., the dosing regimen can be a therapeutic dosing regimen).

As used herein, the term “inhibitor” refers to an agent, condition, or event whose presence, level, degree, type, or form correlates with a decreased level or activity of another agent (i.e., the inhibited agent, or target). In general, an inhibitor may be or include an agent of any chemical class including, for example, small molecules, polypeptides, nucleic acids, carbohydrates, lipids, metals, and/or any other entity, condition or event that shows the relevant inhibitory activity. In some embodiments, an inhibitor may be direct (in which case it exerts its influence directly upon its target, for example by binding to the target); in some embodiments, an inhibitor may be indirect (in which case it exerts its influence by interacting with and/or otherwise altering a regulator of the target, so that level and/or activity of the target is reduced).

As used herein, the terms “patient” or “subject” refer to any organism to which a provided compound(s) or composition(s) described herein are administered in accordance with the present invention e.g., for experimental, diagnostic, prophylactic, and/or therapeutic uses. Typical subjects include animals (e.g., mammals such as mice, rats, rabbits, dogs, cats, non-human primates, and humans; insects; worms; etc.). In some embodiments, a subject may be suffering from a disease (e.g., cancer) or disorder.

As used herein, the terms “prevent,” “prevention,” or “preventing” when used in connection with the occurrence of a disease or disorder, refer to reducing the risk of developing the disease or disorder and/or to delaying the onset of one or more signs or symptoms of the disease or disorder. Prevention may be considered complete when onset of the disease or disorder has been delayed for a predefined period of time.

As used herein, the term “reference” describes a standard or control relative to which a comparison is performed. For example, an agent, animal, cell, individual person, population, sample, sequence or value of interest can be compared with a reference agent, animal, cell, individual person, population, sample, sequence or value. In some embodiments, a reference is tested and/or determined substantially simultaneously with the testing or determination of interest. In some embodiments, a reference is a historical reference or control, optionally embodied in a tangible medium. Typically, as would be understood by one of ordinary skill in the art, a reference is determined or characterized under comparable conditions or circumstances to those under assessment. One of ordinary skill in the art will appreciate when sufficient similarities are present to justify reliance on and/or comparison to a particular possible reference.

With respect to a method of treatment or prophylaxis described herein, a “response” may refer to any beneficial alteration in a subject's condition that occurs as a result of, or correlates with, execution of the method. Such alteration may include stabilization of the condition (e.g., inhibiting deterioration that would have taken place in the absence of the treatment), amelioration of one or more signs or symptoms of the condition, and/or improvement in the prospects for cure of the condition. The alteration may refer to a subject's response or to a tumor's response. Response may be measured according to a wide variety of criteria, including clinical criteria and objective criteria. Techniques for assessing responses include, but are not limited to, assay assessment, clinical examination, positron emission tomatography, X-ray, CT scan, MRI, ultrasound, endoscopy, laparoscopy, presence or level of tumor markers in a sample obtained from a subject, cytology, and/or histology. Regarding a tumor's response, methods and guidelines for assessment are discussed in Therasse et. al. (J. Natl. Cancer Inst., 2000, 92(3):205-216). The exact response criteria can be selected in any appropriate manner, provided that when comparing groups of patients and/or tumors, the groups to be compared are assessed based on the same or comparable criteria for determining a response rate. One of ordinary skill in the art will be able to select appropriate criteria.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, an individual who is “susceptible to” a disease or disorder is at risk for developing the disease or disorder and does not display symptoms of the disease or disorder. In some embodiments, such an individual has not been diagnosed with the disease or disorder. An individual who is susceptible to a disease or disorder can be an individual who has been exposed to conditions associated with development of the disease or disorder (e.g., an individual who is susceptible to cancer may have been exposed to high levels of radiation or carcinogens). In some embodiments, a risk of developing a disease or disorder is a population-based risk (e.g., family members of individuals suffering from the disease or disorder may be susceptible to, or have an elevated risk of developing, the disease or disorder).

As used herein, “symptoms are reduced” when one or more symptoms of a particular disease or disorder are reduced in magnitude (e.g., intensity, severity, etc.) and/or frequency. For purposes of clarity, a delay in the onset of a particular symptom is considered one form of reducing the frequency of that symptom.

As used herein, “improve,” “increase” or “reduce” or grammatical equivalents thereof, indicate values that are relative to a reference measurement, such as a measurement in the same individual prior to initiation of a treatment described herein, or a measurement in a control individual (or multiple control individuals) in the absence of the treatment described herein. In some embodiments, a “control individual” is an individual afflicted with the same form of disease or disorder as an individual being treated.

As used herein, a “therapeutic regimen” is a dosing regimen whose administration across a relevant population is correlated with a desired or beneficial therapeutic outcome.

As used herein, a “therapeutically effective amount” refers to an amount that produces the desired effect for which it is administered. In some embodiments, the term refers to an amount that is sufficient, when administered to a population suffering from or susceptible to a disease or disorder in accordance with a therapeutic dosing regimen, to treat the disease or disorder. In some embodiments, a therapeutically effective amount is one that reduces the incidence and/or severity of, and/or delays onset of, one or more symptoms of the disease or disorder. One of ordinary skill in the art will appreciate that the term “therapeutically effective amount” does not in fact require successful treatment to be achieved in a particular patient/subject. Rather, a therapeutically effective amount may be that amount that provides a particular desired pharmacological response in a significant number of subjects when administered to patients in need of such treatment. Reference to a therapeutically effective amount may be a reference to an amount as measured in one or more specific tissues or cell types (e.g., a tissue or cell type affected by the disease or disorder) or fluids (e.g., blood, saliva, serum, sweat, tears, urine, etc.). One of ordinary skill in the art will appreciate that, in some embodiments, a therapeutically effective amount of a particular agent (e.g., a compound or composition described herein) may be formulated and/or administered in a single dose or in a plurality of doses (e.g., as part of a dosing regimen).

As used herein, the terms “treat,” “treatment,” or “treating” refer to any administration of a therapy that partially or completely alleviates, ameliorates, relives, inhibits, reduces the severity of, and/or reduces the incidence of one or more symptoms, features, and/or causes of a particular disease or disorder. Such treatment may be of a subject who exhibits only early signs or symptoms of the disease or disorder or may be of a subject who exhibits one or more established or advanced signs or symptoms of the relevant disease or disorder. In some embodiments, treatment is of a subject who has been diagnosed as suffering from the relevant disease or disorder. The term “treatment” as used herein, is distinguished from “prophylaxis,” which relates, for example, to delaying onset of one or more signs or symptoms of the particular disease or disorder; to administration to a subject who does not exhibit signs or symptoms of the relevant disease or disorder; and/or to a subject known to have one or more susceptibility factors that are statistically correlated with increased risk of development of the relevant disease or disorder.

Compounds: As described above, provided compounds conform to formula I:

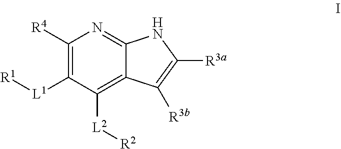

or a pharmaceutically acceptable salt thereof, wherein each of L¹, R¹, L², R², R^{3a}, R^{3b} and R⁴ is as defined above and described in classes and subclasses herein, both singly and in combination.

As used herein, unless otherwise stated, references to formula I also include all subgenera of formula I defined and described herein (e.g., formulae II-a, II-b, II-c, III-a, III-b, III-c, IV-a, IV-b, V-a, V-b, V-c, V-d, VI-a and VI-b).

In some embodiments, L¹ is C₁-C₃ alkylene, —CH═CH—, —C≡C—*, —NH—C(O)—*, —NH—C(O)—CH₂—*, —C(O)—NH—*, —C(O)—NH—CH₂^{-*}, —O—(C₀-C₂ alkylene)-*, —NH—S(O)₂—*, —NH—S(O)₂—CH₂—*, —S(O)₂—NH—*, —S(O)₂—NH—CH₂—*, —NH—(C₀-C₂ alkylene)-*, —O—C(O)—*, —O—C(O)—CH₂—*, —C(O)—O—*, or —C(O)—O—CH₂—*, wherein “*” represents a portion of L¹ bound to R¹.

In some embodiments, L¹ is C₁-C₃ alkylene, —CH═CH—, —CH≡C—*, —NH—C(O)—*, —NH—C(O)—CH₂—*, —C(O)—NH—*, —C(O)—NH—CH₂^{-*}, —O—(C₀-C₂ alkylene)-*, —NH—S(O)₂—*, —NH—S(O)₂—CH₂—*, —S(O)₂—NH—*, —S(O)₂—NH—CH₂—*, —NH—(C₁-C₂ alkylene)-*, —O—C(O)—*, —O—C(O)—CH₂—*, —C(O)—O—*, or —C(O)—O—CH₂—*, wherein “*” represents a portion of L¹ bound to R¹.

In some embodiments, L¹ is a bond, C₁-C₃ alkylene, —CH═CH—, —NH—C(O)—*, —NH—C(O)—CH₂—*, —C(O)—NH—*, —C(O)—NH—CH₂⁻*, —O—(C₁-C₂ alkylene)-*, —NH—S(O)₂—*, —NH—S(O)₂—CH₂—*, —S(O)₂—NH—*, —S(O)₂—NH—CH₂—*, —NH—(C₀-C₂ alkylene)-*, —O—C(O)—*, —O—C(O)—CH₂—*, —C(O)—O—*, or —C(O)—O—CH₂—*, wherein “*” represents a portion of L¹ bound to R¹.

In some embodiments, L¹ is C₁-C₃ alkylene, —CH═CH—, —C≡C—*, —NH—C(O)—*, —NH—C(O)—CH₂—*, —C(O)—NH—*, —C(O)—NH—CH₂^{-*}, —O—(C₁-C₂ alkylene)-*, —NH—S(O)₂—*, —NH—S(O)₂—CH₂—*, —S(O)₂—NH—*, —S(O)₂—NH—CH₂—*, —NH—(C₁-C₂ alkylene)-*, —O—C(O)—*, —O—C(O)—CH₂—*, —C(O)—O—*, or —C(O)—O—CH₂—*, wherein “*” represents a portion of L¹ bound to R¹.

In some embodiments, L¹ is a bond, C₁-C₃ alkylene, —CH═CH—, —C≡C—*, —NH—C(O)—*, —NH—C(O)—CH₂—*, —C(O)—NH—*, —C(O)—NH—CH₂^{-*}, —NH—S(O)₂—*, —NH—S(O)₂—CH₂—*, —S(O)₂—NH—*, —S(O)₂—NH—CH₂—*, —NH—(C₀-C₂ alkylene)-*, —O—C(O)—*, —O—C(O)—CH₂—*, —C(O)—O—*, or —C(O)—O—CH₂—*, wherein “*” represents a portion of L¹ bound to R¹.

In some embodiments, L¹ is —C(O)—NH—CH₂—*, —C(O)—NH—*, C(O)—O—*, or a bond, wherein “*” represents a portion of L¹ bound to R¹. In some embodiments, L¹ is a bond. In some embodiments, L¹ is —C(O)—NH—CH₂—*, wherein “*” represents a portion of L¹ bound to R¹. In some embodiments, L¹ is —C(O)—NH—*, wherein “*” represents a portion of L¹ bound to R¹. In some embodiments, L¹ is C(O)—O—*, wherein “*” represents a portion of L¹ bound to R¹.

In some embodiments, R¹ is halogen, C₁-C₃ alkyl, aryl, heteroaryl, heterocyclyl or carbocyclyl, wherein R¹ is optionally substituted with up to four different substituents. In some embodiments, R¹ is halogen, C₁-C₃ alkyl, aryl, heteroaryl, heterocyclyl or C₄-C₆ carbocyclyl, wherein R¹ is optionally substituted with up to four different substituents.

In some embodiments, R¹ is halogen, C₁-C₃ alkyl, aryl, heteroaryl, heterocyclyl or carbocyclyl, wherein R¹ is optionally substituted with up to four different substituents. In some embodiments, R¹ is halogen, C₁-C₃ alkyl, aryl, heteroaryl, heterocyclyl or C₅-C₆ carbocyclyl, wherein R¹ is optionally substituted with up to four different substituents.

In some embodiments, R¹ is optionally substituted —CH₃, phenyl, cyclohexyl, piperidinyl, azetidinyl, pyridinyl, morpholinyl, pyrimidinyl, pyridazinyl, thiazolyl, tetrahydropyranyl, or pyrazolyl.

In some embodiments, R¹ is halogen. In some embodiments, R¹ is optionally substituted —CH₃. In some embodiments, R¹ is optionally substituted phenyl. In some embodiments, R¹ is optionally substituted cyclohexyl. In some embodiments, R¹ is optionally substituted piperidinyl. In some embodiments, R¹ is optionally substituted azetidinyl. In some embodiments, R¹ is optionally substituted pyridinyl. In some embodiments, R¹ is optionally substituted morpholinyl. In some embodiments, R¹ is optionally substituted pyrimidinyl. In some embodiments, R¹ is optionally substituted pyridazinyl. In some embodiments, R¹ is optionally substituted thiazolyl. In some embodiments, R¹ is optionally substituted tetrahydropyranyl. In some embodiments, R¹ is optionally substituted pyrazolyl.

In some embodiments, R¹ is substituted with —R^{1a}-R^{1b}. In some embodiments, R^{1a} is a bond, —C₁-C₃ alkylene, —S(O)₂, —CH₂—NH—CH₂—, or —C(O)—NH—. In some embodiments, R^{1b} is hydrogen, —F, —N(C₁-C₄ alkyl)₂, —NH(C₁-C₄ alkyl), —NH₂, —O—(C₁-C₄ alkyl), imidazolyl, morpholinyl, pyrimidinyl, adamantyl, piperidinyl, tetrahydropyranyl, tetrahydrofuranyl, pyrrolidinyl, or pyrazolyl, wherein the imidazolyl, morpholinyl, pyrimidinyl, adamantyl, piperidinyl, tetrahydropyranyl, tetrahydrofuranyl, pyrrolidinyl, or pyrazolyl is optionally substituted with up to two independent halogen substituents. In some embodiments, R^{1b} is additionally selected from tetrahydropyrimidinyl.

In some embodiments, R^{1a} is a bond, —C₁-C₃ alkylene, —S(O)₂, —CH₂—NH—CH₂—, or —C(O)—NH— and R^{1b} is hydrogen, —F, —N(C₁-C₄ alkyl)₂, —NH(C₁-C₄ alkyl), —NH₂, —O—(C₁-C₄ alkyl), imidazolyl, morpholinyl, pyrimidinyl, adamantyl, piperidinyl, tetrahydropyranyl, tetrahydrofuranyl, pyrrolidinyl, or pyrazolyl, wherein the imidazolyl, morpholinyl, pyrimidinyl, adamantyl, piperidinyl, tetrahydropyranyl, tetrahydrofuranyl, pyrrolidinyl, or pyrazolyl is optionally substituted with up to two independent halogen substituents. In some embodiments, R^{1b} is additionally selected from optionally substituted tetrahydropyrimidinyl.

In some embodiments, R^{1a} is a bond, —C₁-C₃ alkylene, —S(O)₂ or —CH₂—NH—CH₂—. In some embodiments, R^{1b} is hydrogen, —F, —N(C₁-C₄ alkyl)₂, —NH(C₁-C₄ alkyl), —NH₂, —O—(C₁-C₄ alkyl), imidazolyl, morpholinyl, pyrimidinyl, adamantyl, pyrrolidinyl, or pyrazolyl, wherein the imidazolyl, morpholinyl, pyrimidinyl, adamantyl, piperidinyl, pyrrolidinyl, or pyrazolyl is optionally substituted with up to two independent halogen substituents. In some embodiments, R^{1b} is additionally selected from optionally substituted tetrahydropyrimidinyl.

In some embodiments, R^{1a} is a bond, —C₁-C₃ alkylene, —S(O)₂, or —CH₂—NH—CH₂— and R^{1b} is hydrogen, —F, —N(C₁-C₄ alkyl)₂, —NH(C₁-C₄ alkyl), —NH₂, —O—(C₁-C₄ alkyl), imidazolyl, morpholinyl, pyrimidinyl, adamantyl, pyrrolidinyl, or pyrazolyl, wherein the imidazolyl, morpholinyl, pyrimidinyl, adamantyl, piperidinyl, pyrrolidinyl, or pyrazolyl is optionally substituted with up to two independent halogen substituents. In some embodiments, R^{1b} is additionally selected from optionally substituted tetrahydropyrimidinyl.

In some embodiments, R¹ is —Br, —CH₃, —CH₂—O—CH(CH₃)₂, —C(O)NHCH₃, 1-(2-dimethylaminoethyl)pyrazol-4-yl, 1-(piperidin-4-yl)pyrazol-4-yl, 1-(pyrimidin-2-yl)piperidin-4-yl, 1-(tetrahydrofuran-3-yl)pyrazol-4-yl, 1-(tetrahydrofuran-3-ylmethyl)pyrazol-4-yl, 1-(tetrahydropyran-4-yl)pyrazol-4-yl, 1-(tetrahydropyran-4-ylmethyl)pyrazol-4-yl, 1-(tetrahydropyran-4-ylsulfonyl)azetidin-3-yl, 1-(tetrahydropyran-4-yl sulfonyl)piperidin-4-yl, 1-methylazetidin-3-yl, 1-methylpiperidin-4-yl, 1-methylpyrazol-4-yl, 2-(pyrrolidin-1-yl)pyrimidin-4-yl, 2-aminopyrimidin-4-yl, 4-(1H-imidazol-1-yl)phenyl, 4-(morpholin-4-yl)piperidin-1-yl, 4-(morpholin-4-ylmethyl)cyclohexyl, 4-(morpholin-4-ylmethyl)phenyl, 4-(morpholin-4-ylmethyl)phenyl, 4-aminopyridin-2-yl, 4-fluorophenyl, 5-((((adamant-1-yl)methyl)amino)methyl)pyridin-2-yl, 5-(morpholin-4-ylmethyl)pyridin-2-yl, 5-(morpholin-4-ylsulfonyl)-pyridin-2-yl, 5-aminopyridin-2-yl, 5-fluoropyridin-2-yl, 5-isopropxymethylpyridin-2-yl, 5-(pyrrolidin-1-ylmethyl)pyridin-2-yl, 6-(i sopropylamino)pyrimidin-4-yl, 6-(methylamino)pyrimidin-4-yl, 6-aminopyrimidin-4-yl, 6-methoxypyrimidin-4-yl, 6-methylpyridazin-3-yl, morpholin-2-yl, pyridin-2-yl, tetrahydropyran-4-yl, or thiazol-2-yl. In some embodiments, R¹ is additionally selected from 1-(1,4,5,6-tetrahydropyrimindin-2-yl)piperidin-4-yl.

In some embodiments, R¹ is —CH₃, 1-(2-dimethylaminoethyl)pyrazol-4-yl, 1-(pyrimidin-2-yl)piperidin-4-yl, 1-methylpyrazol-4-yl, 2-(pyrrolidin-1-yl)pyrimidin-4-yl, 2-aminopyrimidin-4-yl, 4-(1H-imidazol-1-yl)phenyl, 4-(morpholin-4-ylmethyl)phenyl, 4-(morpholin-4-ylmethyl)phenyl, 4-aminopyridin-2-yl, 4-fluorophenyl, 5-((((adamant-1-yl)methyl)amino)methyl)pyridin-2-yl, 5-(morpholin-4-ylmethyl)pyridin-2-yl, 5-(morpholin-4-ylsulfonyl)-pyridin-2-yl, 5-aminopyridin-2-yl, 5-fluoropyridin-2-yl, 5-(pyrrolidin-1-ylmethyl)pyridin-2-yl, 6-(isopropylamino)pyrimidin-4-yl, 6-(methylamino)pyrimidin-4-yl, 6-aminopyrimidin-4-yl, 6-methoxypyrimidin-4-yl, 6-methylpyridazin-3-yl, pyridin-2-yl, tetrahydropyran-4-yl, or thiazol-2-yl. In some embodiments, R¹ is additionally selected from 1-(1,4, 5,6-tetrahydropyrimindin-2-yl)piperidin-4-yl.

In some embodiments, when L¹ is a bond, C₁ alkylene, —NH—, —C(O)—O—*, or —O—, R¹ is other than C₁ alkyl or C₁ alkyl substituted with halogen.

In some embodiments, when L¹ is a bond, R¹ is other than cyclopropyl.

In some embodiments, L² is —O—(C₀-C₃ alkylene)-† or —NH—(C₀-C₃ alkylene)-†, wherein “†” represents a portion of L² bound to R², and the alkylene portion of L² if present, is optionally substituted. In some embodiments, L² is —NH— or —O—. In some embodiments, L² is —NH—. In some embodiments, L² is —O—.

In some embodiments, R² is C₁-C₃ alkyl, aryl, heteroaryl, heterocyclyl or carbocyclyl, wherein R² is optionally substituted with up to four different substituents. In some embodiments, R² is optionally substituted with a single substituent selected from —OH and —NH₂. In some embodiments, R² is optionally substituted cyclohexyl, bicyclo[2.2.2]octyl, or bicyclo[1.1.1]pentyl. In some embodiments, R² is optionally substituted cyclohexyl. In some embodiments, R² is 4-aminocyclohexyl, 4-hydroxycyclohexyl, 4-aminobicyclo[2.2.2]octyl, or 4-amino-bicyclo[1.1.1]pentyl. In some embodiments, R² is 4-aminocyclohexyl or 4-hydroxycyclohexyl.

In some embodiments, R^{3a} is optionally substituted —C₁-C₆ alkyl, —C₂-C₆ alkenyl, —C₂-C₆ alkynyl, —(C₁-C₆ alkylene)—O—(C₁-C₆ alkyl), —(C₀-C₃ alkylene)-aryl, —(C₀-C₃ alkylene)-carbocyclyl, —(C₀-C₃ alkylene)-heterocyclyl, or —(C₀-C₃ alkylene)-heteroaryl. In some embodiments, R^{3a} is hydrogen, C₁-C₅ alkyl, C₂-C₅ alkynyl, C₁-C₄ alkylene-O—C₁-C₄ alkyl, C₃-C₆ cycloalkyl, phenyl, benzyl, or an oxygen-containing heterocyclyl, wherein the C₁-C₅ alkyl is optionally substituted with one or more substituents independently selected from halogen, hydroxyl and —OCH₃; and wherein the C₃-C₆ cycloalkyl, phenyl, benzyl, or oxygen-containing containing heterocyclyl is optionally substituted with one or more C₁-C₃ alkyl.

In some embodiments, R^{3a} is hydrogen, C₁-C₅ alkyl, C₂-C₅ alkynyl, or unsubstituted phenyl, wherein the C₁-C₅ alkyl is optionally substituted with one or more substituents independently selected from halogen and hydroxyl.

In some embodiments, R^{1a} is hydrogen, methyl, ethyl, n-propyl, isopropyl, n-butyl, sec-butyl, n-pentyl, —CF₃, hydroxymethyl, methoxymethyl, 1-hydroxyethyl, 2-hydroxyethyl, 1-methoxyethyl, 2-methoxyethyl, 2-fluoroethyl, 1-(fluoromethyl)ethyl, 1-(hydroxymethyl)ethyl, 1-methyl-2-methoxyethyl, trifluoromethyl, 1-hydroxybutyl, 4-hydroxybutyl, 1,4-dihydroxybutyl, 1,4-dimethoxybutyl, 1-(hydroxymethyl)butyl, 1,1,4-trifluorobutyl, n-amyl, sec-amyl, cyclopropyl, 2-ethylcyclopropyl, 2-methylcyclopropyl, cyclobutyl, 3-methylcyclobutyl, cyclopentyl, phenyl, benzyl, oxetan-3-yl, or tetrahydrofuran-2-yl. In some embodiments, R^{1a} is additionally selected from 1-methoxypropyl, pentan-2-yl, pentan-3-yl, 1-hydroxyethyl, 1-ethoxyethyl, 1-methoxybutyl and 1-methoxypropan-2-yl.

In some embodiments, R^{3a} is hydrogen, methyl, ethyl, n-propyl, n-pentyl, isopropyl, sec-butyl, —C≡CH, 1-hydroxyethyl, cyclobutyl, cyclopentyl, or phenyl.

In some embodiments, R^{3b} is hydrogen, halogen, or optionally substituted —C₁-C₄ alkyl. In some embodiments, R^{3b} is hydrogen or —Cl. In some embodiments, R^{3b} is hydrogen. In some embodiments, R^{3b} is —Cl.

In some embodiments, R⁴ is hydrogen, halogen, —C₁-C₄ alkyl, or —O—(C₁-C₄ alkyl), wherein any alkyl portion of R⁴ is optionally substituted. In some embodiments, R⁴ is hydrogen.

In some embodiments, provided compounds are of formula II-a, II-b or II-c:

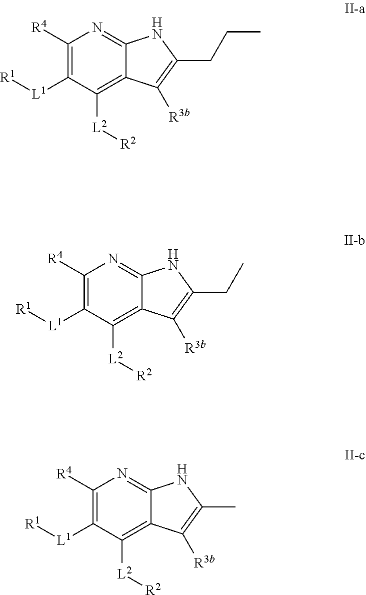

or a pharmaceutically acceptable salt thereof, wherein each of L¹, R¹, L², R², R^{3b} and R⁴ is as defined above and described in classes and subclasses herein, both singly and in combination.

In some embodiments, provided compounds are of formula III-a, III-b and III-c:

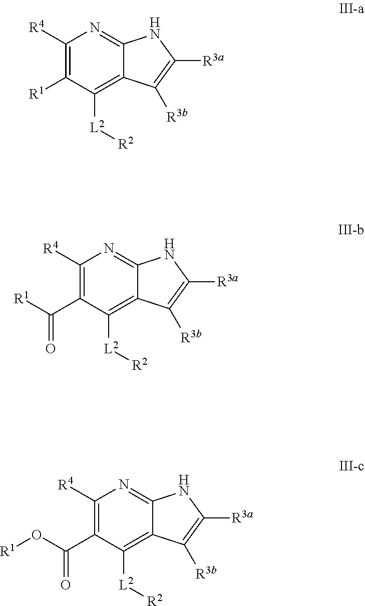

or a pharmaceutically acceptable salt thereof, wherein each of R¹, L², R², R^{3a}, R^{3b} and R⁴ is as defined above and described in classes and subclasses herein, both singly and in combination.

In some embodiments, provided compounds are of formula IV-a and IV-b:

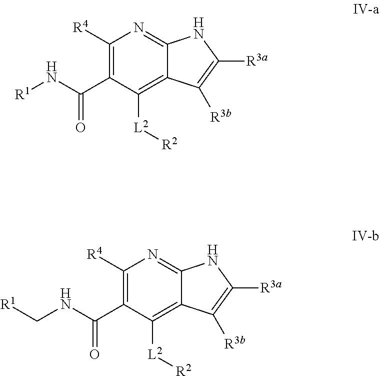

or a pharmaceutically acceptable salt thereof, wherein each of R¹, L², R², R^{3a}, R^{3b} and R⁴ is as defined above and described in classes and subclasses herein, both singly and in combination.

In some embodiments, provided compounds are of formula V-a, V-b, V-c or V-d:

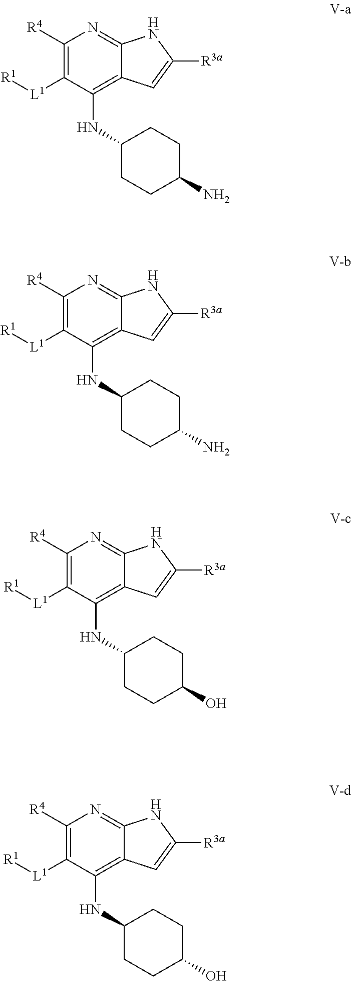

or a pharmaceutically acceptable salt thereof, wherein each of R^{3a} and R⁴ is as defined above and described in classes and subclasses herein, both singly and in combination.

In some embodiments, provided compounds are of formula VI-a or VI-b:

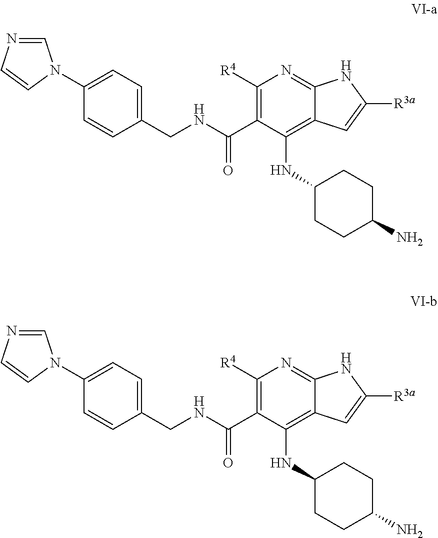

or a pharmaceutically acceptable salt thereof, wherein each of R^{1a} and R⁴ is as defined above and described in classes and subclasses herein, both singly and in combination.

In some embodiments, a provided compound is a compound depicted in Table 1, or a pharmaceutically acceptable salt thereof.

TABLE 1

| | |
|---|---|
| 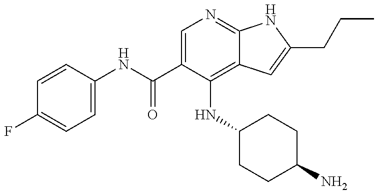 [SMILES: CCCc1cc2c(N[C@H]3CC[C@H](N)CC3)c(C(=O)Nc3ccc(F)cc3)cnc2[nH]1] | 100 |
| 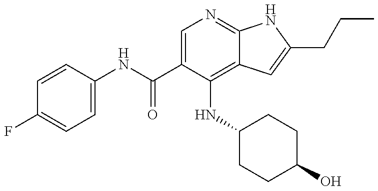 [SMILES: CCCc1cc2c(N[C@H]3CC[C@H](O)CC3)c(C(=O)Nc3ccc(F)cc3)cnc2[nH]1] | 101 |
| 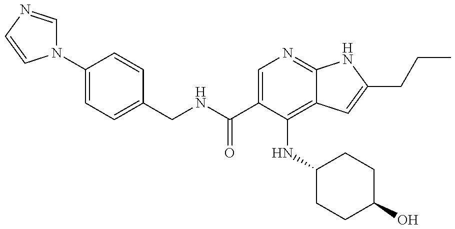 [SMILES: CCCc1cc2c(N[C@H]3CC[C@H](O)CC3)c(C(=O)NCc3ccc(-n4ccnc4)cc3)cnc2[nH]1] | 102 |
| 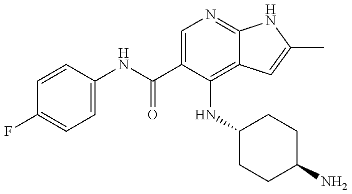 [SMILES: Cc1cc2c(N[C@H]3CC[C@H](N)CC3)c(C(=O)Nc3ccc(F)cc3)cnc2[nH]1] | 103 |
| 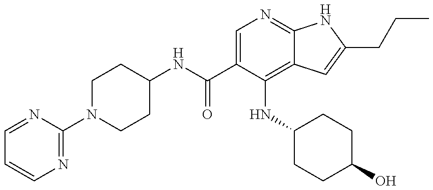 [SMILES: CCCc1cc2c(N[C@H]3CC[C@H](O)CC3)c(C(=O)NC3CCN(c4ncccn4)CC3)cnc2[nH]1] | 104 |
| 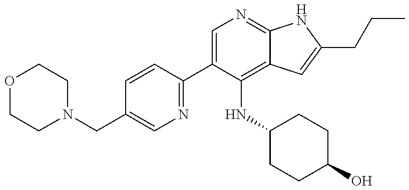 [SMILES: CCCc1cc2c(N[C@H]3CC[C@H](O)CC3)c(-c3ccc(CN4CCOCC4)cn3)cnc2[nH]1] | 105 |
| 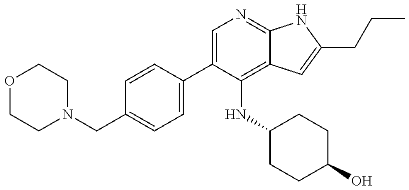 [SMILES: CCCc1cc2c(N[C@H]3CC[C@H](O)CC3)c(-c3ccc(CN4CCOCC4)cc3)cnc2[nH]1] | 106 |
| 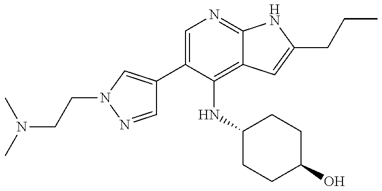 [SMILES: CCCc1cc2c(N[C@H]3CC[C@H](O)CC3)c(-c3cnn(CCN(C)C)c3)cnc2[nH]1] | 107 |
| 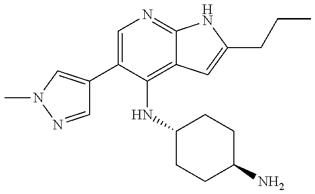 [SMILES: CCCc1cc2c(N[C@H]3CC[C@H](N)CC3)c(-c3cnn(C)c3)cnc2[nH]1] | 108 |
| 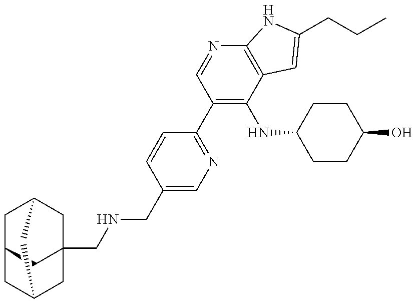 [SMILES: CCCc1cc2c(N[C@H]3CC[C@H](O)CC3)c(-c3ccc(CNCC45C[C@H]6C[C@@H](C4)C[C@@H](C5)C6)cn3)cnc2[nH]1] | 109 |
| 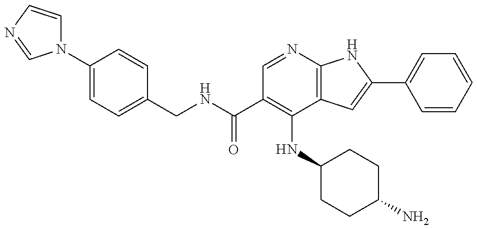 [SMILES: N[C@H]1CC[C@H](Nc2c(C(=O)NCc3ccc(-n4ccnc4)cc3)cnc3[nH]c(-c4ccccc4)cc23)CC1] | 110 |
| 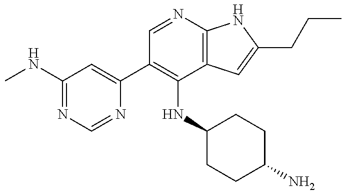 [SMILES: CCCc1cc2c(N[C@H]3CC[C@H](N)CC3)c(-c3cc(NC)ncn3)cnc2[nH]1] | 111 |
| 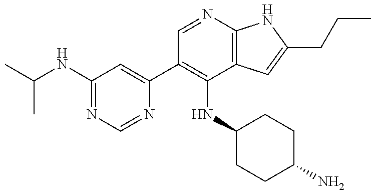 [SMILES: CCCc1cc2c(N[C@H]3CC[C@H](N)CC3)c(-c3cc(NC(C)C)ncn3)cnc2[nH]1] | 112 |
| 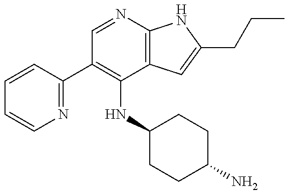 [SMILES: CCCc1cc2c(N[C@H]3CC[C@H](N)CC3)c(-c3ccccn3)cnc2[nH]1] | 113 |
| 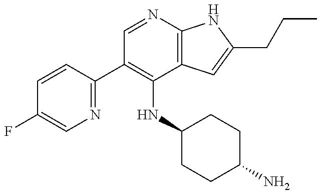 [SMILES: CCCc1cc2c(N[C@H]3CC[C@H](N)CC3)c(-c3ccc(F)cn3)cnc2[nH]1] | 114 |
| 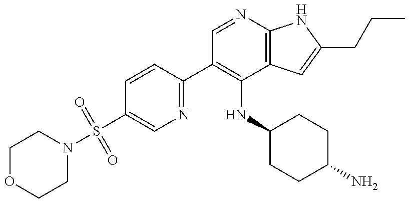 [SMILES: CCCc1cc2c(N[C@H]3CC[C@H](N)CC3)c(-c3ccc(S(=O)(=O)N4CCOCC4)cn3)cnc2[nH]1] | 115 |
| 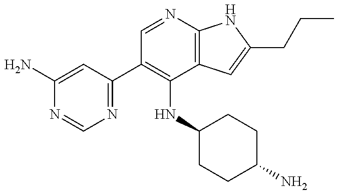 [SMILES: CCCc1cc2c(N[C@H]3CC[C@H](N)CC3)c(-c3cc(N)ncn3)cnc2[nH]1] | 116 |
| 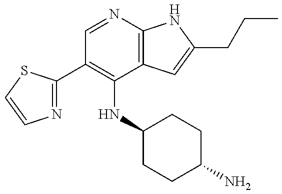 [SMILES: CCCc1cc2c(N[C@H]3CC[C@H](N)CC3)c(-c3nccs3)cnc2[nH]1] | 117 |
| 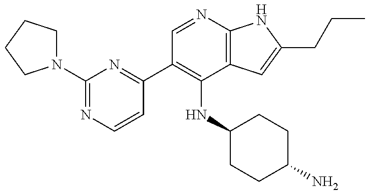 [SMILES: CCCc1cc2c(N[C@H]3CC[C@H](N)CC3)c(-c3ccnc(N4CCCC4)n3)cnc2[nH]1] | 118 |
| 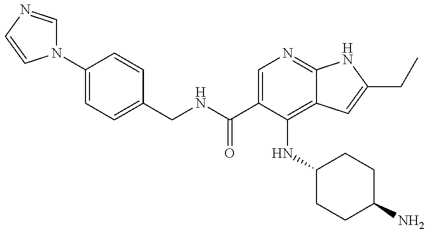 [SMILES: CCc1cc2c(N[C@H]3CC[C@H](N)CC3)c(C(=O)NCc3ccc(-n4ccnc4)cc3)cnc2[nH]1] | 119 |
| 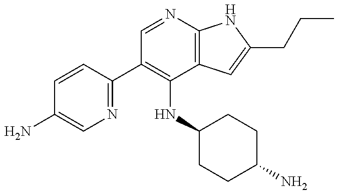 [SMILES: CCCc1cc2c(N[C@H]3CC[C@H](N)CC3)c(-c3ccc(N)cn3)cnc2[nH]1] | 120 |
| 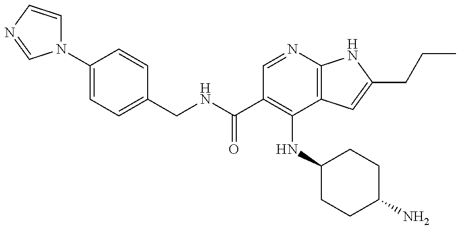 [SMILES: CCCc1cc2c(N[C@H]3CC[C@H](N)CC3)c(C(=O)NCc3ccc(-n4ccnc4)cc3)cnc2[nH]1] | 121 |
| 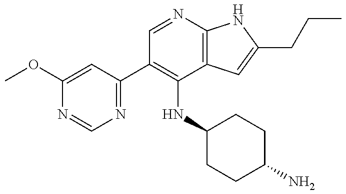 [SMILES: CCCc1cc2c(N[C@H]3CC[C@H](N)CC3)c(-c3cc(OC)ncn3)cnc2[nH]1] | 122 |
| 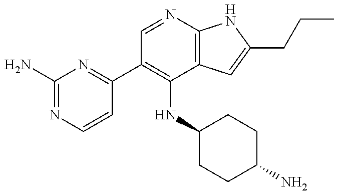 [SMILES: CCCc1cc2c(N[C@H]3CC[C@H](N)CC3)c(-c3ccnc(N)n3)cnc2[nH]1] | 123 |
| 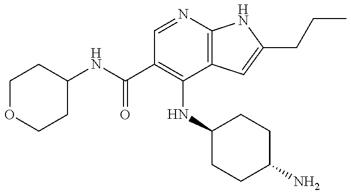 [SMILES: CCCc1cc2c(N[C@H]3CC[C@H](N)CC3)c(C(=O)NC3CCOCC3)cnc2[nH]1] | 124 |
| 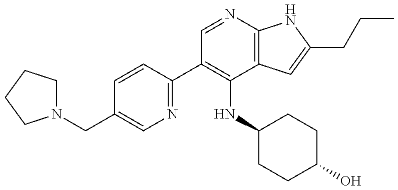 [SMILES: CCCc1cc2c(N[C@H]3CC[C@H](O)CC3)c(-c3ccc(CN4CCCC4)cn3)cnc2[nH]1] | 125 |
| 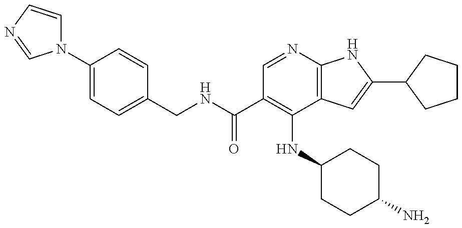 [SMILES: N[C@H]1CC[C@H](Nc2c(C(=O)NCc3ccc(-n4ccnc4)cc3)cnc3[nH]c(C4CCCC4)cc23)CC1] | 126 |
| 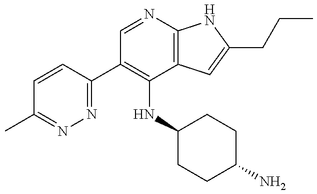 [SMILES: CCCc1cc2c(N[C@H]3CC[C@H](N)CC3)c(-c3ccc(C)nn3)cnc2[nH]1] | 127 |
| 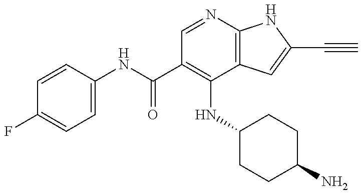 [SMILES: C#Cc1cc2c(N[C@H]3CC[C@H](N)CC3)c(C(=O)Nc3ccc(F)cc3)cnc2[nH]1] | 128 |
| 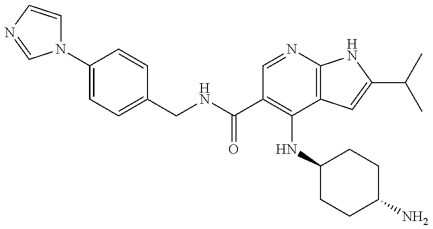 [SMILES: CC(C)c1cc2c(N[C@H]3CC[C@H](N)CC3)c(C(=O)NCc3ccc(-n4ccnc4)cc3)cnc2[nH]1] | 129 |
| 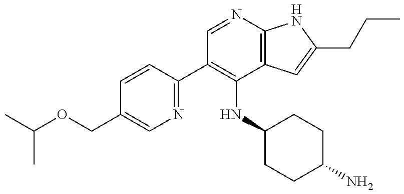 [SMILES: CCCc1cc2c(N[C@H]3CC[C@H](N)CC3)c(-c3ccc(COC(C)C)cn3)cnc2[nH]1] | 130 |
| 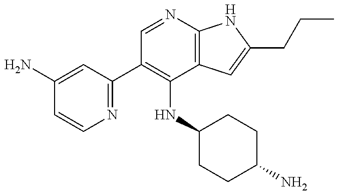 [SMILES: CCCc1cc2c(N[C@H]3CC[C@H](N)CC3)c(-c3cc(N)ccn3)cnc2[nH]1] | 131 |
| 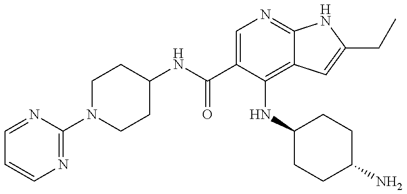 [SMILES: CCc1cc2c(N[C@H]3CC[C@H](N)CC3)c(C(=O)NC3CCN(c4ncccn4)CC3)cnc2[nH]1] | 132 |
| 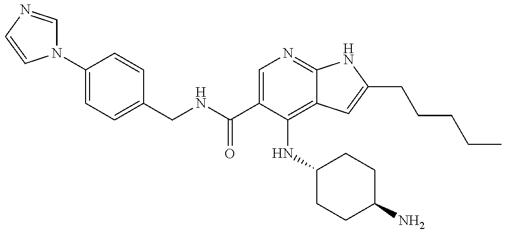 [SMILES: CCCCCc1cc2c(N[C@H]3CC[C@H](N)CC3)c(C(=O)NCc3ccc(-n4ccnc4)cc3)cnc2[nH]1] | 133 |
| 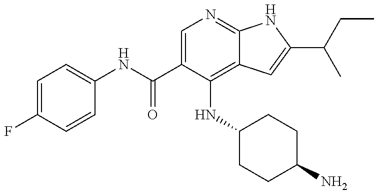 [SMILES: CCC(C)c1cc2c(N[C@H]3CC[C@H](N)CC3)c(C(=O)Nc3ccc(F)cc3)cnc2[nH]1] | 134 |
| 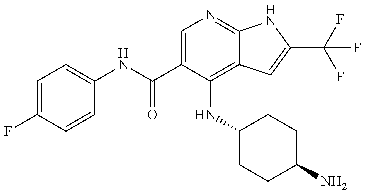 [SMILES: N[C@H]1CC[C@H](Nc2c(C(=O)Nc3ccc(F)cc3)cnc3[nH]c(C(F)(F)F)cc23)CC1] | 135 |
| 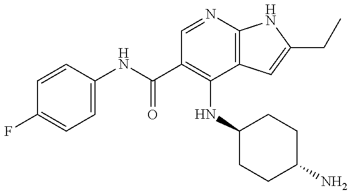 [SMILES: CCc1cc2c(N[C@H]3CC[C@H](N)CC3)c(C(=O)Nc3ccc(F)cc3)cnc2[nH]1] | 136 |
| 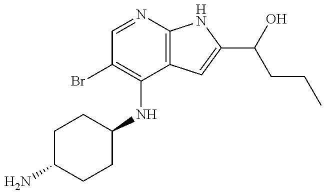 [SMILES: CCCC(O)c1cc2c(N[C@H]3CC[C@H](N)CC3)c(Br)cnc2[nH]1] | 137 |
| 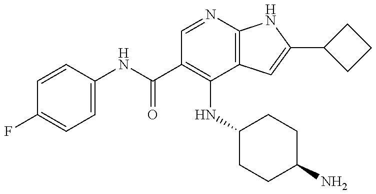 [SMILES: N[C@H]1CC[C@H](Nc2c(C(=O)Nc3ccc(F)cc3)cnc3[nH]c(C4CCC4)cc23)CC1] | 138 |
| 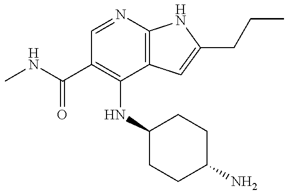 [SMILES: CCCc1cc2c(N[C@H]3CC[C@H](N)CC3)c(C(=O)NC)cnc2[nH]1] | 139 |
| 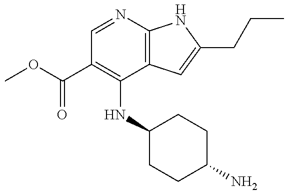 [SMILES: CCCc1cc2c(N[C@H]3CC[C@H](N)CC3)c(C(=O)OC)cnc2[nH]1] | 140 |
| 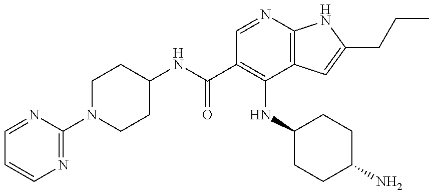 [SMILES: CCCc1cc2c(N[C@H]3CC[C@H](N)CC3)c(C(=O)NC3CCN(c4ncccn4)CC3)cnc2[nH]1] | 141 |
| 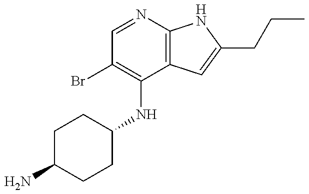 [SMILES: CCCc1cc2c(N[C@H]3CC[C@H](N)CC3)c(Br)cnc2[nH]1] | 142 |
| 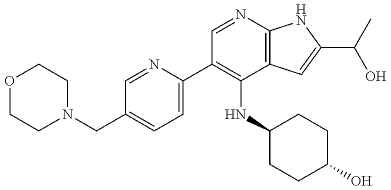 [SMILES: CC(O)c1cc2c(N[C@H]3CC[C@H](O)CC3)c(-c3ccc(CN4CCOCC4)cn3)cnc2[nH]1] | 143 |
| 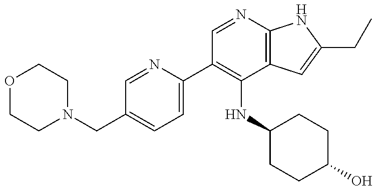 [SMILES: CCc1cc2c(N[C@H]3CC[C@H](O)CC3)c(-c3ccc(CN4CCOCC4)cn3)cnc2[nH]1] | 144 |
| 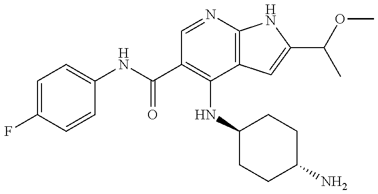 [SMILES: COC(C)c1cc2c(N[C@H]3CC[C@H](N)CC3)c(C(=O)Nc3ccc(F)cc3)cnc2[nH]1] | 145 |
| 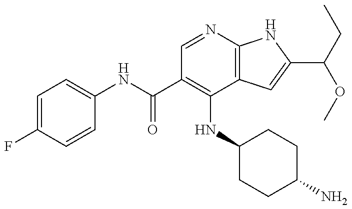 [SMILES: CCC(OC)c1cc2c(N[C@H]3CC[C@H](N)CC3)c(C(=O)Nc3ccc(F)cc3)cnc2[nH]1] | 146 |
| 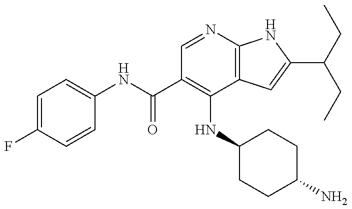 [SMILES: CCC(CC)c1cc2c(N[C@H]3CC[C@H](N)CC3)c(C(=O)Nc3ccc(F)cc3)cnc2[nH]1] | 147 |
| 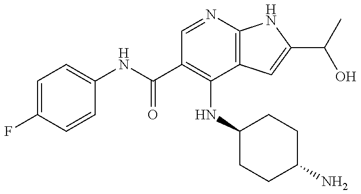 [SMILES: CC(O)c1cc2c(N[C@H]3CC[C@H](N)CC3)c(C(=O)Nc3ccc(F)cc3)cnc2[nH]1] | 148 |
| 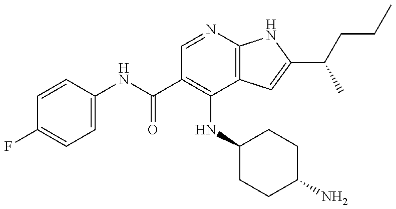 [SMILES: CCC[C@H](C)c1cc2c(N[C@H]3CC[C@H](N)CC3)c(C(=O)Nc3ccc(F)cc3)cnc2[nH]1] | 149 |
| 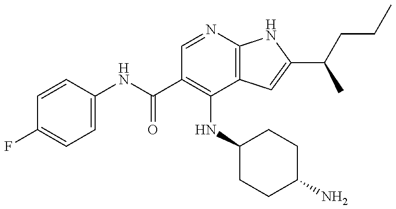 [SMILES: CCC[C@@H](C)c1cc2c(N[C@H]3CC[C@H](N)CC3)c(C(=O)Nc3ccc(F)cc3)cnc2[nH]1] | 150 |
| 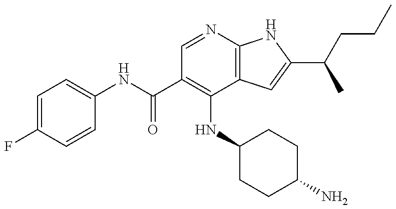 [SMILES: CCC[C@@H](C)c1cc2c(N[C@H]3CC[C@H](N)CC3)c(C(=O)Nc3ccc(F)cc3)cnc2[nH]1] | 151 |
| 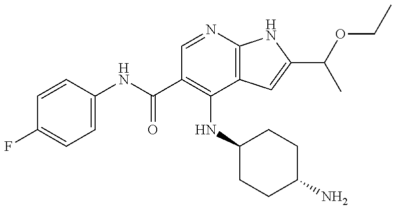 [SMILES: CCOC(C)c1cc2c(N[C@H]3CC[C@H](N)CC3)c(C(=O)Nc3ccc(F)cc3)cnc2[nH]1] | 152 |
| 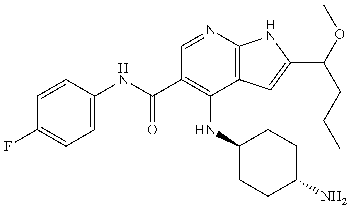 [SMILES: CCCC(OC)c1cc2c(N[C@H]3CC[C@H](N)CC3)c(C(=O)Nc3ccc(F)cc3)cnc2[nH]1] | 153 |
| 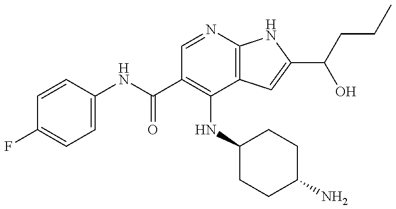 [SMILES: CCCC(O)c1cc2c(N[C@H]3CC[C@H](N)CC3)c(C(=O)Nc3ccc(F)cc3)cnc2[nH]1] | 154 |
| 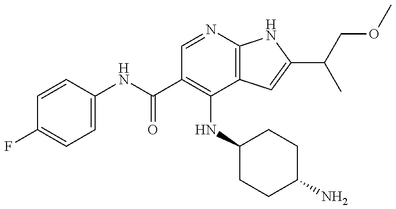 [SMILES: COCC(C)c1cc2c(N[C@H]3CC[C@H](N)CC3)c(C(=O)Nc3ccc(F)cc3)cnc2[nH]1] | 155 |
| 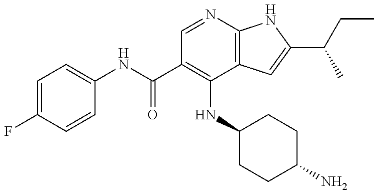 [SMILES: CC[C@H](C)c1cc2c(N[C@H]3CC[C@H](N)CC3)c(C(=O)Nc3ccc(F)cc3)cnc2[nH]1] | 156 |
| 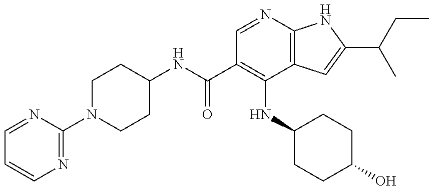 [SMILES: CCC(C)c1cc2c(N[C@H]3CC[C@H](O)CC3)c(C(=O)NC3CCN(c4ncccn4)CC3)cnc2[nH]1] | 157 |
| 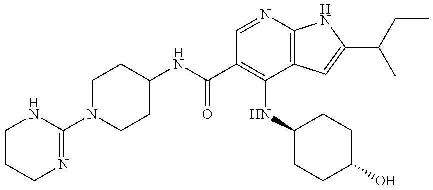 [SMILES: CCC(C)c1cc2c(N[C@H]3CC[C@H](O)CC3)c(C(=O)NC3CCN(C4=NCCCN4)CC3)cnc2[nH]1] | 158 |
| 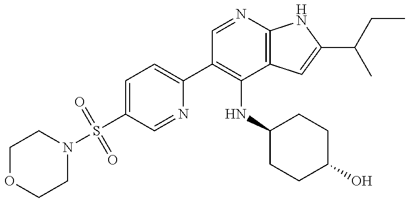 [SMILES: CCC(C)c1cc2c(N[C@H]3CC[C@H](O)CC3)c(-c3ccc(S(=O)(=O)N4CCOCC4)cn3)cnc2[nH]1] | 159 |

The present disclosure provides compositions (e.g., pharmaceutical compositions) comprising any one or more of the compounds described herein.

Exemplary Synthesis of Certain Compounds: Compounds described herein may be prepared using methodologies described herein and/or available in the art. Techniques useful in synthesizing the compounds of the invention are both readily apparent and accessible to one of ordinary skill in the art. The discussion below illustrates certain of the diverse methods available for use in assembling the compounds described herein, but it is not intended to define the scope of reactions or reaction sequences that are useful in preparing the compounds of the invention.

The present compounds can be generally prepared according to Schemes 1-4.

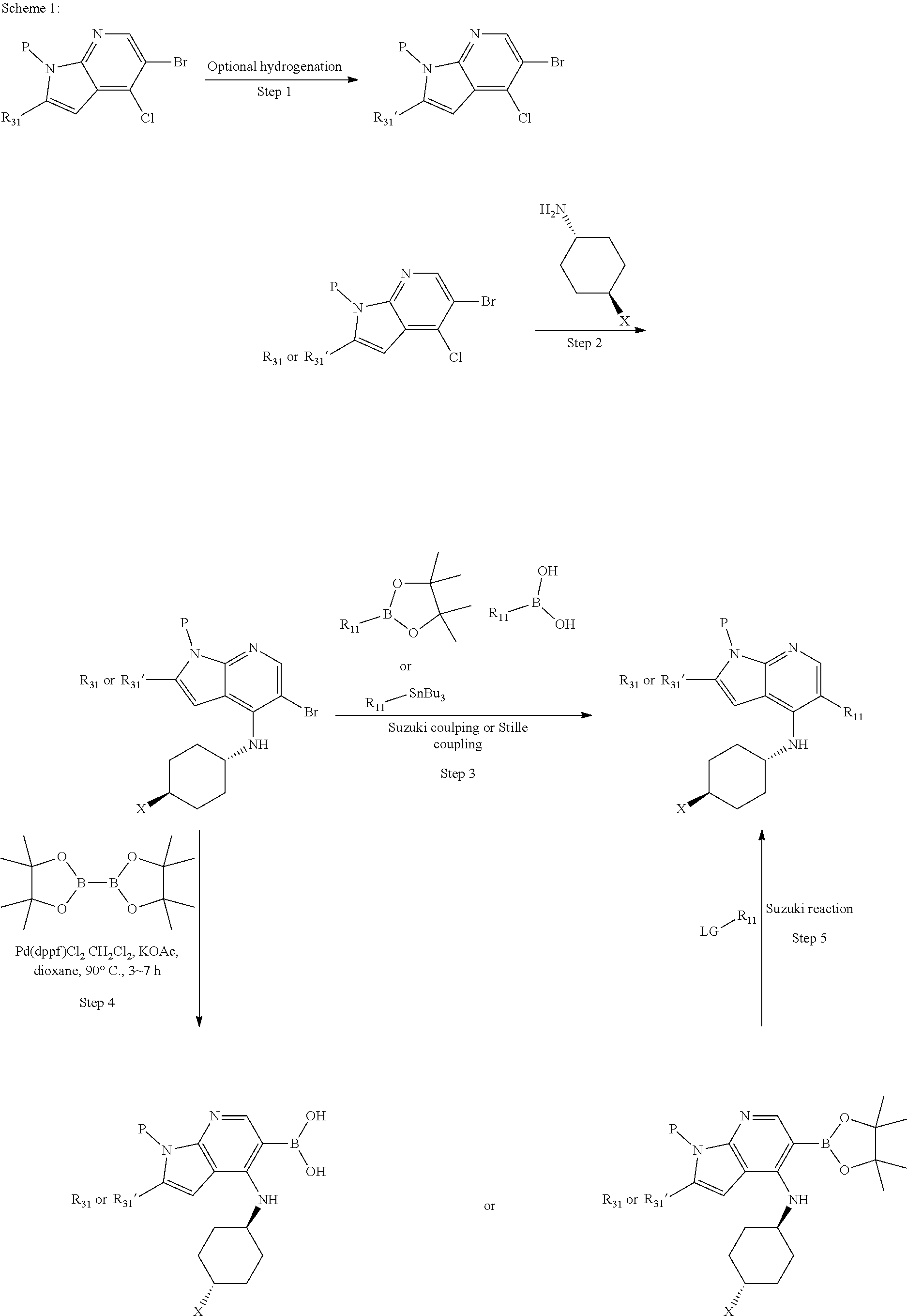

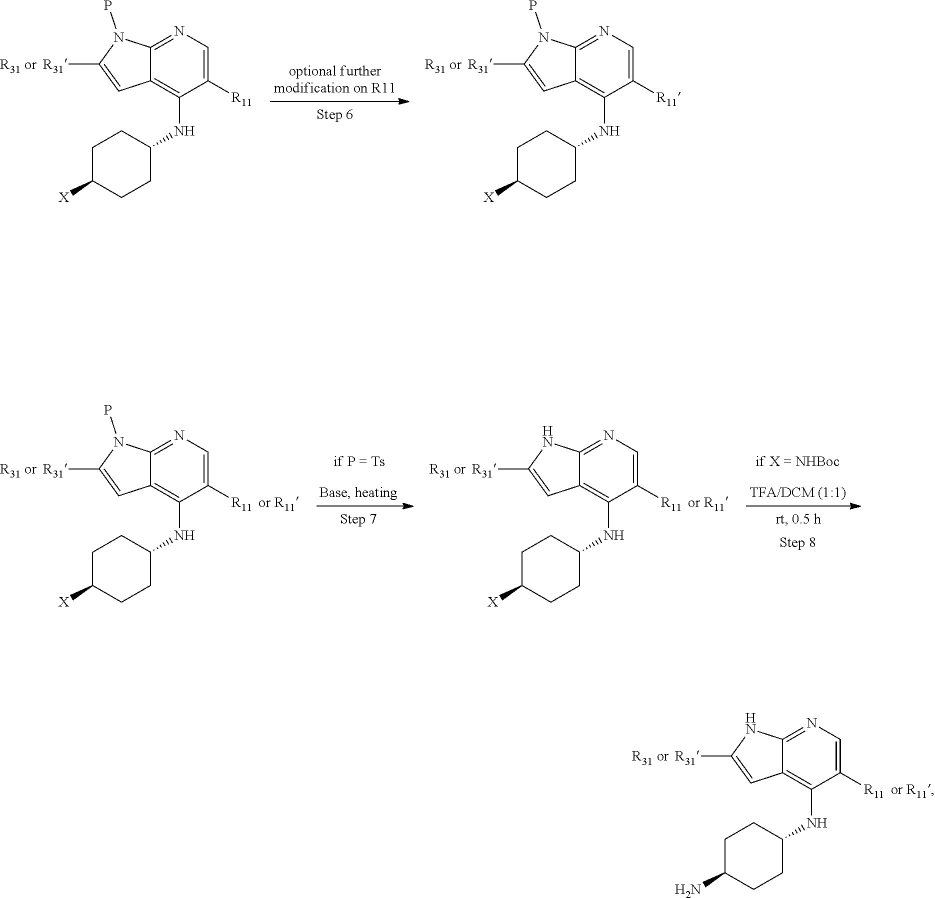

wherein:
R₁₁ is halogen, C₁-C₃ alkyl, aryl, heteroaryl, heterocyclyl or carbocyclyl optionally substituted with up to four independently selected substituents;
R₁₁′ is C₁-C₃ alkyl, aryl, heteroaryl, heterocyclyl or carbocyclyl substituted with one to four independently selected substituents;
R₃₁ is optionally substituted —C₂-C₆ alkenyl, —(C₁-C₆ alkylene)—O—(C₁-C₆ alkyl), —(C₀-C₃ alkylene)-aryl, —(C₀-C₃ alkylene)-carbocyclyl, —(C₀-C₃ alkylene)-heterocyclyl, or —(C₀-C₃ alkylene)-heteroaryl;
R₃₁′ is optionally substituted —C₁-C₆ alkyl;
P is H or a tosyl group;
X is OH, NH₂ or NHBoc; and
LG is a leaving group (e.g. Cl, Br, I, etc.).

Each of the aforementioned synthetic steps may be performed sequentially with isolation of each intermediate performed after each step. Alternatively, each of Step 1, Step 2, Step 3, Step 4, Step 5, Step 6, Step 7, and Step 8, as depicted in Scheme 1 above, may be performed in a manner whereby no isolation of one or more intermediates is performed. It will be readily apparent to one of ordinary skill in the art that additional steps may be performed to accomplish particular protection group and/or deprotection strategies.

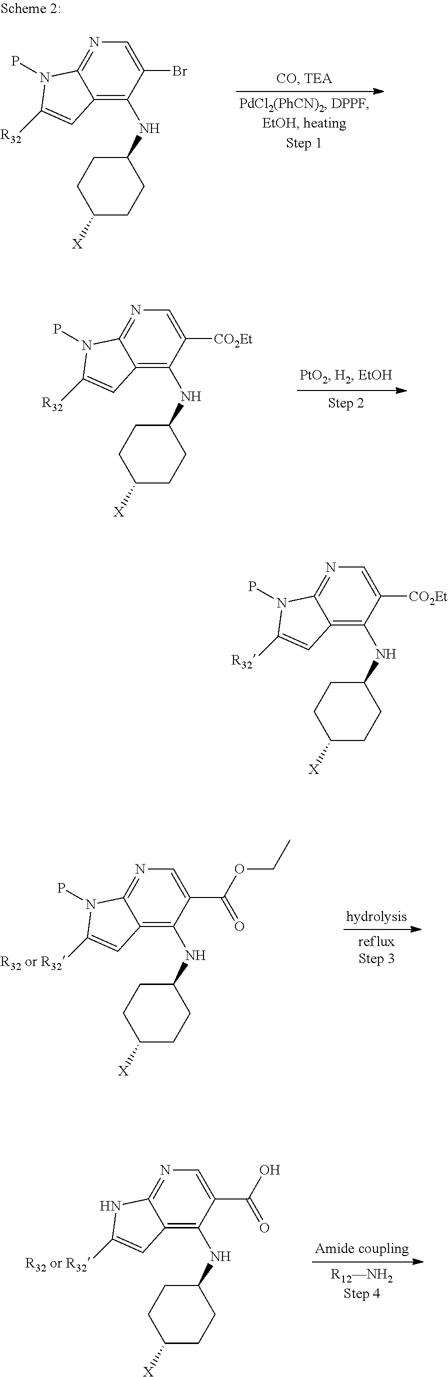

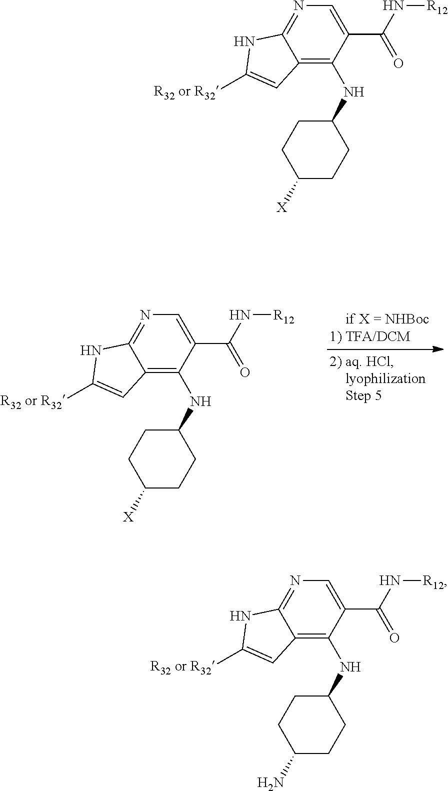

wherein: R₁₂ is C₁-C₃ alkyl, aryl, heteroaryl, heterocyclyl or carbocyclyl optionally substituted with up to four independently selected substituents; R₃₂ is optionally substituted —C₂-C₆ alkenyl, —(C₁-C₆ alkylene)—O—(C₁-C₆ alkyl), —(C₀-C₃ alkylene)-aryl, —(C₀-C₃ alkylene)-carbocycl yl, —(C₀-C₃ alkyl ene)-heterocyclyl, or —(C₀-C₃ alkylene)-heteroaryl; R₃₂′ is optionally substituted —C₁-C₆ alkyl; P is H or tosyl group; and X is OH or NHBoc.

Each of the aforementioned synthetic steps may be performed sequentially with isolation of each intermediate performed after each step. Alternatively, each one of Steps 1-5 as depicted in Scheme 2 may be performed in a manner whereby no isolation of one or more intermediates is performed. Furthermore, it will be apparent to one of ordinary skill in the art that additional steps may be performed to accomplish particular protection group and/or deprotection strategies.

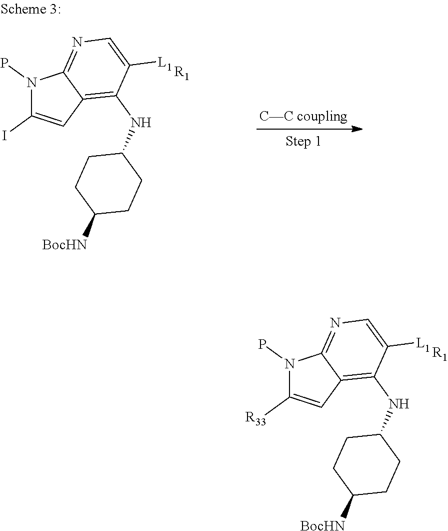

C—C coupling conditions include but are not limited to Suzuki reaction, Sonagashira reaction, photochemistry, Heck reaction, Stille reaction, Negishi reaction, and CF3 installation method.

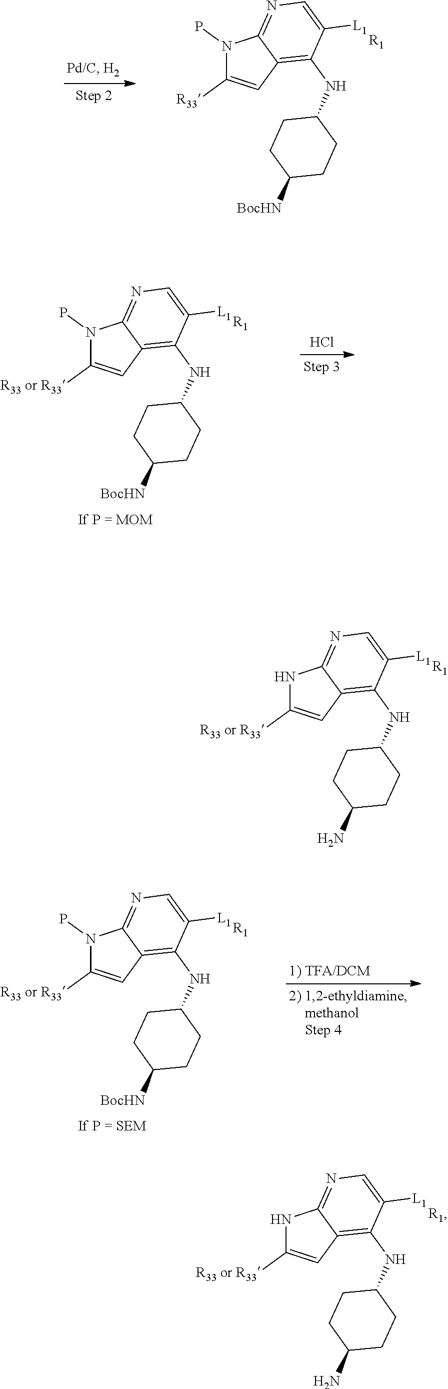

wherein:
L¹ and R¹ are as defined in Formula I;
R₃₃ is optionally substituted -C₂-C₆ alkenyl, —(C₁-C₆ alkylene)—O—(C₁-C₆ alkyl), —(C₀-C₃ alkylene)-aryl, —(C₀-C₃ al kyl ene)-carbocyclyl, —(C₀-C₃ alkylene)-heterocyclyl, or —(C₀-C₃ alkylene)-heteroaryl;
R₃₃′ is optionally substituted —C₁-C₆ alkyl; and
P is MOM or SEM.

Each of the aforementioned synthetic steps may be performed sequentially with isolation of each intermediate performed after each step. Alternatively, each of Step 1, Step 2, Step 3, and Step 4 as depicted in Scheme 3 may be performed in a manner whereby no isolation of one or more intermediates is performed. Furthermore, it will be readily apparent to one of ordinary skill in the art that additional steps may be performed to accomplish particular protection group and/or deprotecti on strategies.

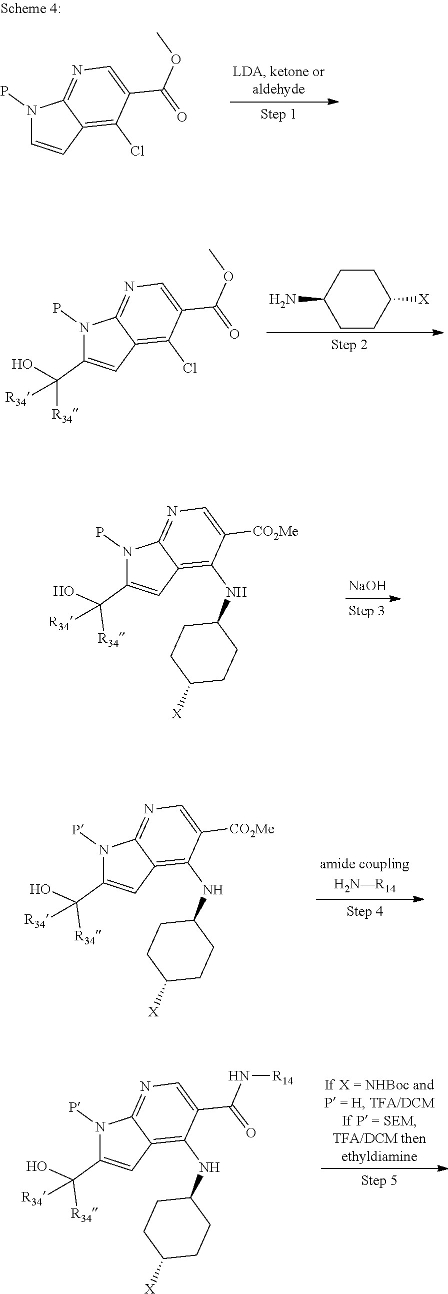

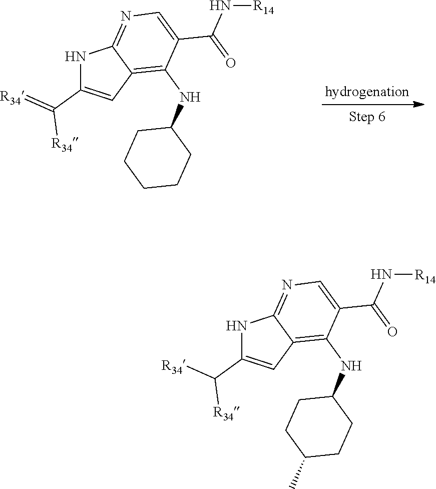

wherein:
R₁₄ is optionally substituted C₁-C₃ alkyl, aryl, heteroaryl, heterocyclyl or carbocyclyl with up to four different substituents;
R₃₄′ and R₃₄″ are independently optionally substituted —C₁-C₆ alkyl, —C₂-C₆ alkenyl, —C₂-C₆ alkynyl, —(C₁-C₆ alkylene)—O—(C₁-C₆ alkyl), —(C₀-C₃ alkylene)-aryl, —(C₀-C₃ alkylene)-carbocyclyl, —(C₀-C₃ alkylene)-heterocyclyl, or —(C₀-C₃ alkylene)-heteroaryl;
P is SEM or tosyl group;
P′ is SEM or H;
X is NHBoc or OH; and
X′═NH₂ or OH.

Each of the aforementioned synthetic steps may be performed sequentially with isolation of each intermediate performed after each step. Alternatively, each of Step 1, Step 2, Step 3, Step 4, Step 5, and Step 6 as depicted in Scheme 4 may be performed in a manner whereby no isolation of one or more intermediates is performed. Furthermore, it will be apparent to one of ordinary skill in the art that additional steps may be performed to accomplish particular protection group and/or deprotection strategies.

Any of the steps of the aforementioned syntheses may be performed to prepare the desired final product. In other embodiments, two, three, four, five, or more sequential steps may be performed to prepare an intermediate or the desired final product.

It will be appreciated by one of ordinary skill in the art that certain starting materials depicted in Schemes 1-4 may be readily interchanged with other starting materials or reagents to provide additional compounds of formula I. Such substitutions could be made with routine experimentation.

TAM Kinases: TAM family expression is heterogeneous among macrophage subsets, being mostly restricted to anti-inflammatory M2 macrophages, which contribute significantly to the immunosuppression present in the tumor microenvironment. Through inhibition of the TAM kinases on tumor-infiltration macrophages, the immunosuppressive environment can be reduced by repolarizing M2 macrophages. This can result in increased effector killer immune cell function and tumor regression. Current CD8T and NK cell-directed immunotherapies have shown promising efficacy but only in a limited percentage of patients. The reason for this limitation is not well understood but it is hypothesized that the immunosuppressive environment is inhibiting efficacy. Since M2 macrophages contribute significantly to this environment, reversal of the M2 phenotype may increase the number of patients who will respond to CD8T and NK cell-directed immunotherapies. Therefore, TAM kinase inhibitors can be used in oncology indications where checkpoint inhibitors have shown limited efficacy and have high myeloid infiltration, such as pancreatic ductal adenocarcinoma, ovarian cancer, triple-negative breast cancer, glioblastoma and colorectal cancer.

In addition, abnormally increased (relative to that amount observed in a reference non-cancer cell or tissue) MERTK expression has been reported in multiple human cancer types including leukemias, lymphomas, gastric cancers, prostate cancers, breast cancers, pituitary adenomas, NSCLCs, melanomas, glioblastomas, ovarian cancers and rhabdomyosarcomas. The overexpression of MERTK in cancer cells results in increased survival and resistance to apoptosis resulting in oncogenesis. In some embodiments, compounds of the present invention inhibit all three members of the TAM family. In some embodiments, inhibition of the TAM family kinases in oncology clinical indications where one or more of the TAM family members are overexpressed can result in tumor burden reduction and increased patient survival.

Compounds previously reported to have potent inhibitory activity against one or more TAM kinases have limitations. For example, some of these inhibitors may be potent against only one or two of the TAM kinases. Such partial activity may be sufficient for a direct anti-cancer effect in a cancer with dependency on a specific TAM kinase. However, given the redundancy of the TAM kinases' role in driving the M2 immunosuppressive functional state, without potent inhibition of all three kinases, the uninhibited kinase(s) can likely compensate for the inhibited kinases.

The present disclosure identifies that when CD14⁺ cells are isolated from patient tumors heterogeneity is observed in the expression of the three TAM kinases amongst patients, but in the majority of patients studied all three TAMs are expressed. Many compounds have undesirable off-target effects. Compounds which report TAM inhibition, also potently inhibit kinases that are required for immune system maintenance and renewal (RET, KIT, MET, FLT3, etc.). Inhibition of these kinases results in cytopenia and a lack of immune system in patients. These activities negate the possibility of using these compounds in a therapeutic treatment predicated upon an immune-based mechanism of action.

The present disclosure encompasses treating a cancer in a patient in need thereof, which patient expresses or overexpresses one or more TAM kinases (e.g., TYRO3, AXL and MERTK). A patient may be (or have been) determined to express such kinases by a process comprising obtaining a sample from the patient and determining whether the kinases (e.g., TYRO3, AXL and/or MERTK) are present and, optionally, at what level(s). The patient can be treated by administering, to the patient, a therapeutically effective amount of a compound of the present invention, a prodrug thereof, or a pharmaceutically acceptable salt thereof.

The term “specific,” when used herein with reference to an agent having an activity (e.g., a compound disclosed herein, a prodrug thereof, or a pharmaceutically acceptable salt thereof), is understood by one of ordinary skill in the art to mean that the agent discriminates between potential target entities or states. For example, an agent binds “specifically” to its target if it binds preferentially with that target in the presence of one or more competing alternative targets. In many embodiments, specific interaction depends upon the presence of a particular structural feature of the target entity (e.g., an epitope, a cleft, a binding site). It is to be understood that specificity need not be absolute; specificity may be evaluated relative to that of a binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, the binding agent binds with higher on-rate, lower off-rate, increased affinity, decreased dissociation, and/or increased stability to its target entity as compared with the competing alternative target(s).

A provided compound may have a comparable level of activity against each of TYRO3, AXL and MERTK or it may show activity above a particular reference level with respect to each of TYRO3, AXL and MERTK. A provided compound may show specificity for one or more of, or for each of, TYRO3, AXL and MERTK. In some embodiments, a provided compound shows more specificity for one or more TAM kinases (e.g., for one or more of TYRO3, AXL and MERTK) relative to other kinases. In some embodiments, a provided compound shows more specificity for each of TYRO3, AXL and MERTK relative to other kinases.

In various embodiments, a provided compound is considered to be specific for a given kinase or set of kinases when: (a) it shows at least 2×, 5×, 10×, 20×, 30×, 40×, 50×, 60×, 70×, 80×, 90×, 100× or more activity for the specific kinase(s) than for one or more appropriate comparator kinase(s); (b) it shows at least 2-fold, 3-fold, 4-fold, 5-fold, 10-fold, 15-fold, 20-fold, 50-fold, 100-fold or more activity for the specific kinase(s) than for one or more appropriate comparator kinase(s); and/or (c) when it shows at least 101%, 105%, 110%, 120%, 130%, 140%, 150%, 200%, 300%, 400%, 500% or more activity for the specific kinase(s) than for one or more appropriate comparator kinase(s). In any of the embodiments just described (a-c), the activity of the specific kinase(s) may be the activity of one or more TAM kinases relative to one or more non-TAM kinases, of one or more of TYRO3, AXL and MERTK relative to one or more kinases other than TYRO3, AXL and MERTK, or of one or more of TYRO3, AXL, and MERTK relative to one another.

Specificity can be characterized by “specific binding,” which refers to an ability to discriminate between possible binding partners in the environment in which binding is to occur. A binding agent (e.g., a compound described herein or a pharmaceutically acceptable salt thereof) that interacts with one particular target (e.g., a TAM kinase) when other potential targets are present is said to “bind specifically” to the target with which it interacts. Specific binding can be assessed by detecting or determining a degree of association between the binding agent and its target or a degree of dissociation of a binding agent-target complex. Specific binding can also be assessed by detecting or determining an ability of the binding agent to compete in an alternative interaction between its target and another entity. Any such detections or determinations can be performed across a range of concentrations.

An activity with respect to which a provided compound demonstrates specificity is, or includes, one or more of binding activity, inhibitory activity, ability to compete with an alternative ligand for binding (e.g., a reference compound or composition), and/or other effects on the kinase, etc. An activity with respect to which a provided compound demonstrates specificity may be assessed, for example, as an IC₅₀. In various embodiments, an activity with respect to which a provided compound demonstrates specificity may be assessed by competitive inhibition; by determining a inhibitory constant; and/or by determining kinase inhibition potency.

FLT3: FLT3 (FMS-like tyrosine kinase 3, also known as Flk2) is a member of the type III RTK family and plays an important role in the proliferation and differentiation of hematopoietic stem cells. Activating mutation or overexpression of this receptor is found in acute myeloid leukemia (AML), acute lymphocytic leukemia (ALL), mastocytosis and gastrointestinal stromal tumor (GIST). In addition to activating mutations, autocrine or paracrine ligand stimulation of overexpressed wild type FLT3 can contribute to the malignant phenotype. The ligand for FLT3 is expressed by the marrow stromal cells and other cells and synergizes with other growth factors to stimulate proliferation of stem cells, progenitor cells, dendritic cells, and natural killer cells.

Methods of Use: Compounds and/or compositions (e.g., pharmaceutical compositions) provided herein have a variety of uses, including uses in research, analysis, and/or therapy.

Also provided herein are methods for inhibiting a TAM kinase, the method including a step of contacting the TAM kinase with an amount of a provided compound or pharmaceutically acceptable salt thereof that inhibits the kinase. The kinase can be one or more of the TAM kinases TYRO3, AXL and/or MERTK. In some embodiments, the kinase is TYRO3; in others, it is AXL; and in others, it is MERTK.

In some embodiments, compounds of the present disclosure, prodrugs thereof, pharmaceutically acceptable salts thereof, and compositions (e.g., pharmaceutical compositions containing the compound(s), prodrug(s), and/or salt(s) are for use in medicine. In some embodiments, these compounds provide a method of treating and/or preventing (e.g., delaying the onset of) a disease or disorder responsive to inhibition of a TAM kinase (e.g., TYRO3, AXL and/or MERTK). The disease can be cancer. In some embodiments, compounds of the present disclosure can be used in methods of enhancing an effect, the methods including a step of administering to a subject an amount of a provided compound, thereby treating or preventing the condition. The provided compound can be a compound described herein or a pharmaceutically acceptable salt thereof, and the amount can be a therapeutically effective amount. In some embodiments, compounds of the present disclosure and pharmaceutically acceptable salts thereof are useful in the treatment of cancers or as therapeutics for the treatment of cancers, such as leukemias, lymphomas, gastric cancers, prostate cancers, breast cancers, pituitary adenomas, NSCLCs, melanomas, glioblastomas, ovarian cancers and rhabdomyosarcomas. In some embodiments, compounds of the present disclosure are for use in preventing a cancer by reducing a subject's risk of developing the cancer or delaying the onset of one or more signs or symptoms of the cancer including any or more of the types of cancer just listed.

Pharmaceutical Compositions: In another aspect, the present invention provides pharmaceutical compositions that include a compound of formula I, a prodrug thereof, a pharmaceutically acceptable salt thereof, any of which can be included in combination with a pharmaceutically acceptable carrier (e.g., an excipient).

The pharmaceutical compositions can include optical isomers, diastereomers, or pharmaceutically acceptable salts of the compounds disclosed herein. The compound of formula I that is included in the pharmaceutical composition may be covalently attached to a carrier moiety. Alternatively, the compound of formula I included in the pharmaceutical composition is not covalently linked to a carrier moiety.

A compound of the invention, a prodrug thereof, or a pharmaceutically acceptable salt thereof can be administered alone (i.e., the method or use constitutes giving a subject a single type of compound, prodrug, or salt) or can be coadministered to the subject (i.e., the method or use constitutes giving a subject two or more types of the compound, prodrug, and/or salt (i.e., a plurality of compounds, prodrugs, and/or salts)). Coadministration is meant to include simultaneous or sequential administration of the plurality of compounds, prodrugs, and/or salts. The methods described here can also include a step of administering, when desired, other active substances, including the additional therapeutic agents discussed below, and the invention encompasses pharmaceutical compositions that include a single type of compound, prodrug, or salt; a plurality of compounds, prodrugs, and/or salts; and either a single type of compound, prodrug, or salt, or a plurality thereof in combination with another active substance (e.g., another active pharmaceutical ingredient).

Combinations: Any one or more of the compounds described herein may also be used in combination with one or more additional therapeutic agents. The present disclosure thus provides, in a further aspect, a combination comprising a compound described herein or a pharmaceutically acceptable salt thereof together with at least one additional therapeutic agent. In some embodiments, the additional therapeutic agent is a provided compound. In some embodiments, the additional therapeutic agent includes a boron atom.

When a provided compound is used in combination with a second therapeutic agent active against the same disease or disease state, the dose of each compound may differ from that when the compound is used alone. Appropriate doses will be readily appreciated by one of ordinary skill in the art. It will be appreciated that the amount of a provided compound required for use in treatment will vary with the nature of the condition being treated and the age and the condition of the patient and will be ultimately at the discretion of the attendant physician or veterinarian.

It will be appreciated that, in some embodiments, provided compounds are utilized in combination with (e.g., administered to subjects receiving) therapy (e.g., standard of care therapy) for the treatment of cancer. In some such embodiments, the cancer is one or more of breast cancer, ovarian cancer, glioblastoma, pancreatic ductal adenocarcinoma, NSCLC, colorectal cancer, leukemia, lymphoma, gastric cancer, prostate cancer, pituitary adenoma, melanoma, or rhabdomyosarcoma. In some embodiments, a provided compound and/or pharmaceutical composition is administered to a cancer (e.g., cancer cells) resistant to a checkpoint inhibitor. In some embodiments, the cancer is associated with elevated levels of myeloid infiltration compared to a reference cell or population of reference cells (e.g., a normal cell or population of cells).

Alternatively or additionally, in some embodiments, provided compounds are utilized in combination with (e.g., administered to subjects receiving) immunotherapy. In some embodiments, such immunotherapy comprises or consists of checkpoint inhibitor therapy, vaccine therapy (e.g., cancer vaccine therapy), and/or cell therapy (e.g., CAR-T therapy and/or CAR-NK therapy). In some embodiments, provided combinations are administered to subjects who have or will receive antibody therapy, cell therapy (e.g., CAR-T therapy and/or CAR-NK therapy), chemotherapy, hormone therapy (e.g., that reduces level of hormone and/or hormone receptor and/or inhibits hormone-receptor interaction or one or more downstream effects thereof), radiation therapy, and/or surgical therapy.

Formulations: Compounds of the present disclosure can be prepared and administered in a wide variety of oral, parenteral, and topical dosage forms. Thus, the compounds of the present disclosure can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds of the present disclosure can be administered transdermally. It is also envisioned that multiple routes of administration (e.g., intramuscular, oral, transdermal) can be used to administer the compounds of the present disclosure. Accordingly, the present disclosure also provides pharmaceutical compositions comprising a pharmaceutically acceptable carrier and one or more compounds of the present disclosure.

The pharmaceutical compositions can include pharmaceutically acceptable carriers that are either solid or liquid. Solid form preparations include powders, tablets, pills, capsules, cachets, suppositories, and dispersible granules. A solid carrier can be one or more substances that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material (e.g., a compound of the invention or pharmaceutical composition can be surrounded by a capsule, which is thus in association with the compound or composition). Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In some embodiments, suitable carriers for parenteral administration will be selected for human administration.

In particular, carriers for parenteral administration include aqueous solutions of dextrose, saline, pure water, ethanol, glycerol, glycerol formal, polyethylene glycol, propylene glycol, peanut oil, sesame oil, polyoxyethylene-block polymers, pyrrolidine, N-methyl pyrrolidione, and the like. Ampoules are convenient unit dosages. The compounds of the present disclosure can also be incorporated into liposomes or administered via transdermal pumps or patches. Pharmaceutical admixtures suitable for use in the present disclosure include those described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

Pharmaceutical compositions of the present invention may additionally include components to provide sustained release and/or comfort (e.g., high molecular weight, anionic mucomimetic polymers, gelling polysaccharides, and finely-divided drug carrier substrates). Such components are discussed in U.S. Pat. Nos. 4,911,920; 5,403,841; 5,212,162; and 4,861,760. The entire contents of these patents are incorporated herein by reference in their entirety for all purposes.

Effective Dosage: Pharmaceutical compositions provided by the present disclosure include compositions wherein the active ingredient is contained in a therapeutically effective amount, i.e., in an amount effective to achieve its intended purpose. The actual amount effective for a particular application will depend, inter alfa, on the condition being treated.

The dosage and frequency (single or multiple doses) of compound(s) administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods, compounds, and compositions of the present disclosure.

For any compound described herein, the therapeutically effective amount can be initially determined from, or analyzed in part using, cell culture assays.

Therapeutically effective amounts for use in humans may also be determined from animal models (e.g., a dose for humans can be formulated to achieve a concentration that has been found to be effective in animals). The dosage in humans can be adjusted by, for example, monitoring kinase inhibition, other markers, and/or signs and symptoms of the disease or disorder being treatedand adjusting the dosage upwards or downwards.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under the circumstances is reached. In some embodiments, the dosage range is 0.001% to 10% w/v. In some embodiments, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the patient's disease.

### EXAMPLES

The following analytical instruments were used in the synthesis and analysis of the compounds of the invention:

| | |
|---|---|
| LCMS | Shimadzu UFLC MS: LCMS-2020 |
| | Agilent Technologies 1200 series MS: Agilent Technologies 6110 |
| | Agilent Technologies 1200 series MS: LC/MSD VL |
| NMR | BRUKER AVANCE 111/400 MHz |
| Prep-HPLC | Gilson GX-281 systems: instruments GX-A, GX-B, GX-C, GX-D, |
| | GX-E, GX-F, GX-G and GX-H |

### Example 1: Synthesis of Intermediates

### A. tert-butyl((1r,4r)-4-((5-bromo-2-propyl-1-tosyl-1H-pyrrolo[2,3-b]pyridine-4-yl)amino) cyclohexyl)carbamate

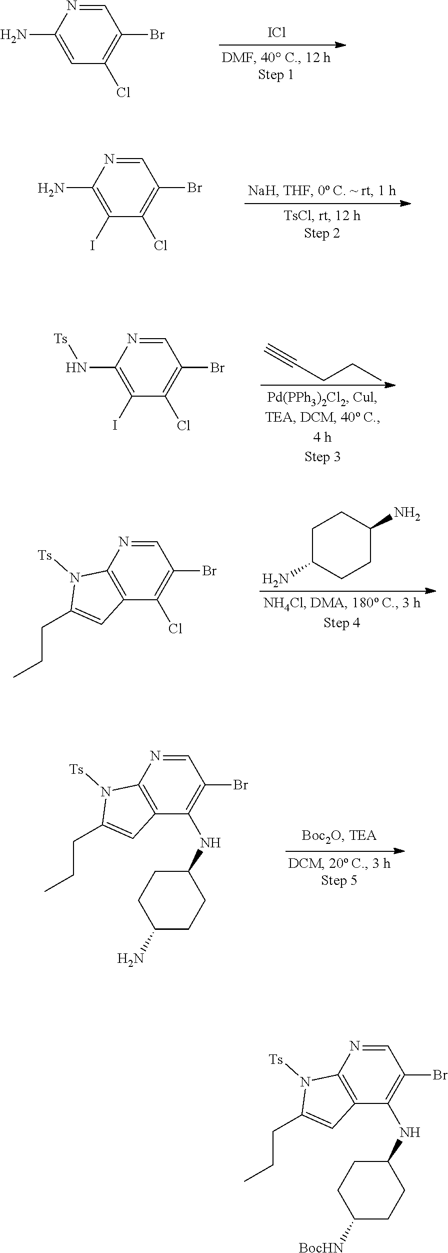

### Step 1: 5-bromo-4-chloro-3-iodopyridin-2-amine

To a solution of 5-bromo-4-chloro-pyridin-2-amine (15 g, 72 mmol) in N,N-dimethylformamide (150 mL) was added dropwise a solution of iodine monochloride (1 M in dichloromethane, 260 mL). The reaction mixture was stirred at 40° C. for 12 hours. Upon completion, the mixture was poured into water (500 mL) then extracted with dichloromethane (3×500 mL). Organic layers were combined, washed with brine (2×500 mL), dried over anhydrous sodium sulfate, filtered and then concentrated in vacuo to give a red oil. The red oil was poured into ice water (500 mL), filtered and then concentrated in vacuo to afford 5-bromo-4-chloro-3-iodopyridin-2-amine (22 g, 95% purity, 87% yield) as a red solid.

### Step 2: N-(5-bromo-4-chloro-3-iodopyridin-2-yl)-4-methylbenzenesulfonamide

To a solution of sodium hydride (7.2 g, 0.18 mol, 60% purity) in tetrahydrofuran (200 mL) at 0° C., was added dropwise a solution of 5-bromo-4-chloro-3-iodopyridin-2-amine (20 g, 60 mmol) in tetrahydrofuran (200 mL). The mixture was then warmed up to room temperature and stirred for 1 hour. A solution of 4-methylbenzenesulfonyl chloride (34 g, 0.18 mol) in tetrahydrofuran (100 mL) was added dropwise at room temperature. The mixture was stirred at this temperature for another 12 hours. Upon completion, the mixture was poured into water (100 mL), extracted with ethyl acetate (2×500 mL). Organic layers were combined, washed with brine (2×100 mL), dried over anhydrous sodium sulfate, filtered and concentrated in vacuo. The residue was purified by silica gel chromatography to yield N-(5-bromo-4-chloro-3-iodopyridin-2-yl)-4-methylbenzenesulfonamide (19 g, 65% yield) as a gray solid.

### Step 3: 5-bromo-4-chloro-2-propyl-1-tosyl-1H-pyrrolo[2,3-b]pyridine

A solution of N-(5-bromo-4-chloro-3-iodopyridin-2-yl)-4-methylbenzenesulfonamide (17 g, 35 mmol), pent-1-yne (2.6 g, 38 mmol, 3.8 mL), Pd(PPh₃)₂C1₂ (2.5 g, 3.5 mmol), copper(I) iodide (1.3 g, 7.0 mmol) and triethylamine (11 g, 0.10 mol) in dichloromethane (170 mL) was stirred at 40° C. for 4 hours under nitrogen. Upon completion, the mixture was poured into water (100 mL) and extracted with dichloromethane (2×200 mL). Organic layers were combined, washed with brine (2×100 mL), dried over anhydrous sodium sulfate, filtered and concentrated in vacuo. The residue was purified by silica gel chromatography to yield 5-bromo-4-chloro-2-propyl-1-tosyl-1H-pyrrolo[2,3-b]pyridine (13 g, 79% yield) as a yellow solid.

### Step 4: (1r,4r-N45-bromo-2-propyl-1-tosyl-1H-pyrrolo[2,3-b]pyridin-4-yl) cyclohexane-1,4-diamine

To a mixture of 5-bromo-4-chloro-2-propyl-1-tosyl-1H-pyrrolo[2,3-b]pyridine (5.0 g, 12 mmol) and (1r,4r-cyclohexane-1,4-diamine (6.7 g, 58 mmol) in N,N-dimethylacetamide (50 mL) was added ammonium chloride (0.13 g, 2.3 mmol). The mixture was heated at 180° C. for 3 hours. The reaction mixture was then poured into ice water (200 mL) and then filtered. The filter residue was concentrated in vacuo to yield (1r,4r-N1-(5-bromo-2-propyl-1-tosyl-1H-pyrrolo[2,3-b]pyridin-4-yl)cyclohexane-1,4-diamine (5 g, crude) as a greenish blue solid.

### Step 5: tert-butyl(1r,4r)-4((5-bromo-2-propyl-1-tosyl-1H-pyrrolo[2,3-b]pyridin-4-yl) amino)cyclohexyl)carbamate

To a solution of (1r,4r-N¹-(5-bromo-2-propyl-1-tosyl-1H-pyrrolo[2,3-b]pyridin-4-yl) cyclohexane-1,4-diamine (5.0 g, 9.9 mmol) in dichloromethane (250 mL) was added triethylamine (1.3 g, 13 mmol) and di-tent-butyl dicarbonate (3.2 g, 15 mmol) under nitrogen. The mixture was stirred at room temperature for 3 hours. Upon completion, the mixture was added to water (100 mL) and extracted with dichloromethane (2×250 mL). The combined organic phases were washed with brine (2×100 mL), dried over anhydrous sodium sulfate and then concentrated in vacuo. The residue was purified by silica gel chromatography to give tert-butyl((1r,4)-4-((5-bromo-2-propyl-1-tosyl-1H-pyrrolo[2,3-b]pyridin-4-yl)amino)cyclo-hexyl)carbamate (3.4 g, 49% yield over two steps) as a yellow solid.

### B. tert-butyl ((1r,4r)-4((5-bromo-2-((E)-prop-1-en-1-yl)-1-tosyl-1H-pyrrolo[2,3-b]pyridine -4-yl)amino)cyclohexyl)carbamate

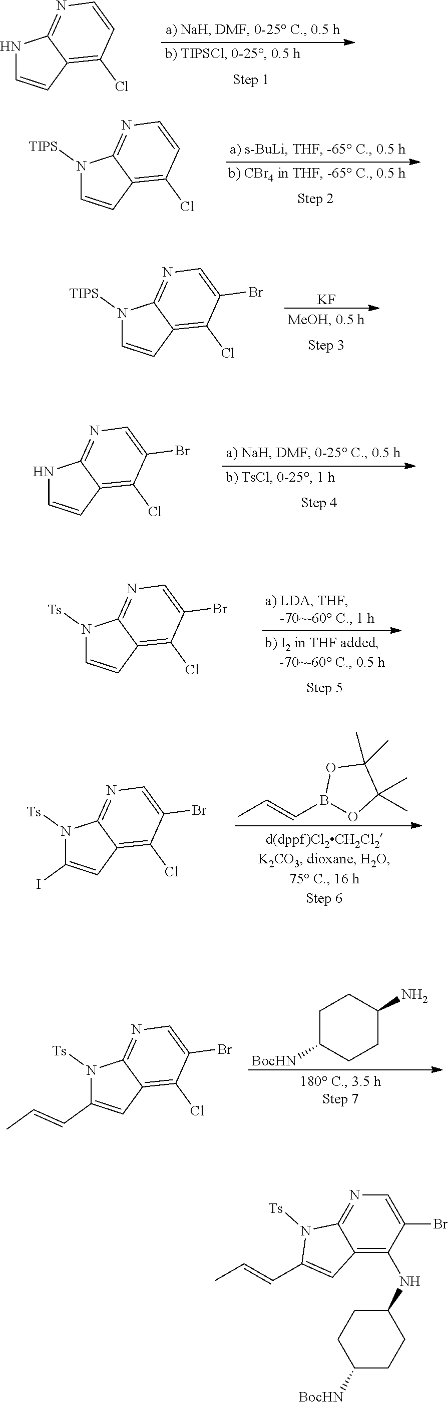

### Step 1: 4-chloro-1-(triisopropylsilyl)-1H-pyrrolo[2,3-b]pyridine

To a solution of 4-chloro-1H-pyrrolo[2,3-b]pyridine (30 g, 196 mmol) in dimethylformamide (300 mL) was added sodium hydride (10 g, 255 mmol, 60% purity) at 0° C. under nitrogen. The mixture was stirred at 25° C. for 0.5 hour, then cooled to 0° C. Triisopropyl-chlorosilane (49 g, 255 mmol) was added to the mixture at 0° C. The reaction was stirred at room temperature for 0.5 hour. Upon completion, the mixture was quenched with water (500 mL),extracted with ethyl acetate (3×300 mL). The organic layer was washed by water (200 mL), brine (2×200 mL), dried over anhydrous sodium sulfate and concentrated in vacuo. The residue was purified by silica gel chromatography to afford 4-chloro-1-(triisopropylsilyl)-1H-pyrrolo[2,3-b]pyridine (56.8 g, 93% yield) as a colorless oil.

### Step 2: 5-bromo-4-chloro-1-(triisopropylsilyl)-1H-pyrrolo[2,3-b]pyridine

To a solution of 4-chloro-1-(triisopropylsilyl)-1H-pyrrolo[2,3-b]pyridine (20 g, 64 mmol) in anhydrous tetrahydrofuran (300 mL) was added sec-Butyllithium (1.3 M, 110 mL) at −65˜−60° C. under nitrogen. The mixture was stirred at −65° C. for 0.5 hour. Then carbon tetrabromide (26 g, 77 mmol) in anhydrous tetrahydrofuran (20 mL) was added dropwise to the solution. The mixture was stirred at −65° C. for another 0.5 hour. Upon completion, the mixture was quenched with aqueous saturated ammonium chloride solution (300 mL). The mixture was extracted with ethyl acetate (2×200 mL). Organic layers were combined, washed with water (100 mL), brine (100 mL) sequentially, dried over anhydrous sodium sulfate, filtered and concentrated in vacuo. The residue was purified by silica gel chromatography to afford 5-bromo-4-chloro-1-(triisopropylsilyl)-1H-pyrrolo[2,3-b]pyridine (28.8 g, 92% yield) as a yellow oil.

### Step 3: 5-bromo-4-chloro-1H-pyrrolo[2,3-b]pyridine

To a solution of 5-bromo-4-chloro-1-(triisopropylsilyl)-1H-pyrrolo[2,3-b]pyridine (29 g, 59 mmol) in methanol (300 mL) was added potassium fluoride (3.8 g, 65 mmol). The mixture was stirred at room temperature for 0.5 hour. Upon completion, the mixture was filtered and the filtered cake was washed with dichloromethane. The white solid was dried under vacuum to give the title compound (8.3 g, 60% yield), which was used for the next step directly.

### Step 4: 5-bromo-4-chloro-1-tosyl-1H-pyrrolo[2,3-b]pyridine

To a solution of 5-bromo-4-chloro-1H-pyrrolo[2,3-b]pyridine (15.7 g, 68 mmol) in dimethylformamide (150 mL) was added sodium hydride (3.5 g, 88 mmol, 60% purity) at 0° C. under nitrogen. The mixture was stirred at room temperature for 0.5 hour, then treated with 4-methylbenzene-1-sulfonyl chloride (21 g, 108 mmol). The mixture was stirred at room temperature for another one hour. Upon completion, the mixture was poured into ice water (400 mL). The precipitation was filtered. The resulting solid was dissolved into ethanol (80 mL). The mixture was heated to 100° C., then cooled to 40-50° C. The slurry was filtered to afford 5-bromo-4-chloro-1-tosyl-1H-pyrrolo[2,3-b]pyridine (22.5 g, 86% yield) as a white solid.

### Step 5: 5-bromo-4-chloro-2-iodo-1-tosyl-1H-pyrrolo[2,3-b]pyridine

To a solution of 5-bromo-4-chloro-1-tosyl-1H-pyrrolo[2,3-b]pyridine (8.5 g, 22 mmol) in anhydrous tetrahydrofuran (100mL) was added lithium diisopropylamide (3.5 g, 33 mmol) at −70 to −60° C. under nitrogen. The mixture was stirred at −70° C. for 1 hour, then iodine (8.4 g, 33 mmol) in tetrahydrofuran (50 mL) was added to the reaction mixture at −70° C. to −60° C. The mixture was stirred at −70° C. for 0.5 hour. Upon completion, the mixture was quenched by aqueous saturated sodium thiosulfate (100 mL), and the mixture was extracted with ethyl acetate (2×150 mL). Organic layers were combined, washed with water, brine sequentially, dried over anhydrous sodium sulfate, filtered and concentrated in vacuo. The residue was purified by silica gel chromatography. The purified product was washed with petroleum ether/ethyl acetate=50:1. The precipitated product was filtered to afford 5-bromo-4-chloro-2-iodo-1-tosyl-1H-pyrrolo [2,3-b]pyridine (5.9 g, 47% yield) as a white solid.

### Step 6: (E)-5-bromo-4-chloro-2-(prop-1-en-1-yl)-1-tosyl-1H-pyrrolo[2,3-b]pyridine

To a mixture of 5-bromo-4-chloro-2-iodo-1-tosyl-1H-pyrrolo[2,3-b]pyridine (2.3 g, 4.5 mmol), potassium carbonate (1.9 g, 13.5 mmol) and Pd(dppf)Cl₂CH₂Cl₂ (0.37 g, 0.045 mmol) in a mixture of dioxane (40 mL) and water (8 mL) was added 4,4,5,5-tetramethyl-2-[(E)-prop-1-enyl]-1,3,2-dioxaborolane (0.79 g, 4.7 mmol) under nitrogen. The mixture was stirred at 75° C. for 16 hours. Upon completion, the mixture was diluted with ethyl acetate (200 mL), washed with water (80 mL) and brine (80 mL) sequentially, dried over anhydrous sodium sulfate, filtered and concentrated in vacuo. The residue was purified by silica gel chromatography to afford (E)-5-bromo-4-chloro-2-(prop-1-en-1-yl)-1-tosyl-1H-pyrrolo [2,3-b]pyridine (1.7 g, 76% yield) as a white solid.

### Step 7: tert-butyl ((1r,4r)-4-((5-bromo-24(E)-prop-1-en-1-yl)-1-tosyl-1H-pyrrolo[2,3-b]pyridine-4-yl)amino)cyclohexyl)carbamate

A mixture of (E)-5-bromo-4-chloro-2-(prop-1-en-1-yl)-1-tosyl-1H-pyrrolo [2,3-b]pyridine (0.5 g, 1.2 mmol) and tert-butyl N-(4-aminocyclohexyl)carbamate (1.0 g, 4.7 mmol) was heated to 180° C. for 3.5 hours. Upon completion, the mixture was diluted with a mixture of ethyl acetate:methanol (1:1) (100 mL). The mixture was sonicated and filtered. The filtrate was concentrated in vacuo. The residue was purified by silica gel chromatography to give tert-butyl ((1r,4r)-4-((5-bromo-2-((E)-prop-1-en-1-yl)-1-tosyl-1H-pyrrolo[2,3-b]pyridine -4-yl)amino)cyclohexyl)carbamate (0.22 g, 11% yield) as a white solid.

### C. (1r,4r)-4—O—bromo-2-propyl-1-tosyl-1H-pyrrolo[2,3-b]pyridin-4-yl)amino) cyclohexanol

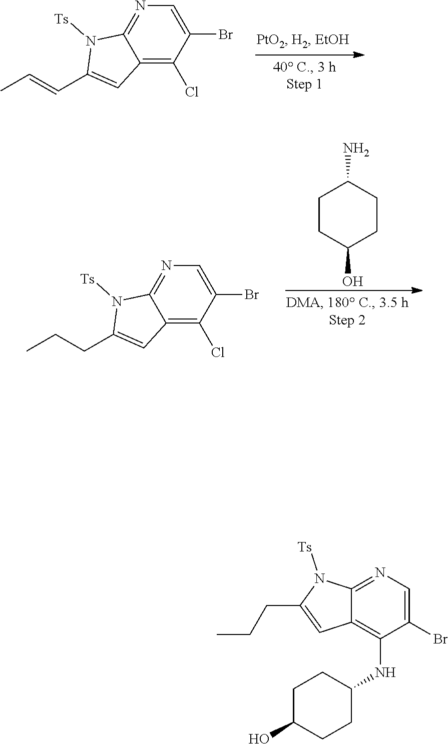 [SMILES: C/C=C/c1cc2c(Cl)c(Br)cnc2n1S(=O)(=O)c1ccc(C)cc1 ; CCCc1cc2c(Cl)c(Br)cnc2n1S(=O)(=O)c1ccc(C)cc1 ; N[C@H]1CC[C@H](O)CC1 ; CCCc1cc2c(N[C@H]3CC[C@H](O)CC3)c(Br)cnc2n1S(=O)(=O)c1ccc(C)cc1]

### Step 1: 5-bromo-4-chloro-2-propyl-1-tosyl-1H-pyrrolo[2,3-b]pyridine

To a solution of 5-bromo-4-chloro-2-[(E)-prop-1-enyl]-1-(p-tolylsulfonyl) pyrrolo[2,3-b]pyridine (800 mg, 2 mmol) in ethanol (EtOH; 20 mL) was added PtO₂ (platinum dioxide; 427 mg, 2 mmol) under N₂. The suspension was degassed under vacuum and purged with H₂ several times. The mixture was stirred under H₂ (50 psi) at 40° C. for 3 hours. Upon completion, the mixture was diluted with dichloromethane (DCM; 50 mL) and filtered. The filtrate was concentrated in vacuo. The residue was purified by column chromatography to give 5-bromo-4-chloro-2-propyl-1-tosyl-1H-pyrrolo[2,3-b]pyridine (700 mg, 87% yield) as a white solid.

### Step 2: (1r,4r)-4-((5-bromo-2-propyl-1-tosyl-1H-pyrrolo[2,3-b]pyridin-4-yl)amino) cyclohexanol

This step was carried out similar to Step 7 of Example 1, part B substituting 1-hydroxy-4-aminocyclohexyl for tert-butyl N-(4-aminocyclohexyl)carbamate.

### D. 5-bromo-4-chloro-2-methyl-1-tosyl-1H-pyrrolo[2,3-b]pyridine

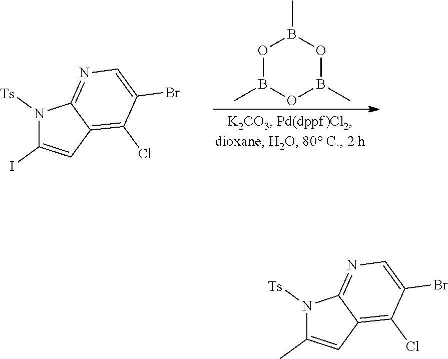

To a mixture of 5-bromo-4-chloro-2-iodo-1-tosyl-1H-pyrrolo[2,3-b]pyridine (1.0 g, 2.0 mmol), potassium carbonate (0.81g, 5.9 mmol) and Pd(dppf)Cl₂ (0.14 g, 0.2 mmol) in a mixture of dioxane (20 mL) and water (5 mL) was added 2,4,6-trimethyl-1,3,5,2,4,6-trioxatriborinane (0.50 g, 2.0 mmol, 50% purity) under nitrogen. The mixture was stirred at 80° C. for 2 hours. Upon completion, the mixture was diluted with ethyl acetate (100 mL) and washed with water (40 mL), brine (2 ×20 mL) sequentially. The organic layer was dried over anhydrous sodium sulfate, filtered and concentrated in vacuo. The residue was purified by silica gel chromatography to afford 5-bromo-4-chloro-2-methyl-1-tosyl-1H-pyrrolo[2,3-b] pyridine (0.56 g, 1.1 mmol, 55% yield) as a white solid.

### E. tert-butyl ((1r,4r)-4-((5-((4-(1H-imidazol-1-yl)benzyl)carbamoyl)-2-iodo-1-(methoxymethyl)-1H-pyrrolo[2,3-b]pyridin-4-yl)amino)cyclohexyl)carbamate

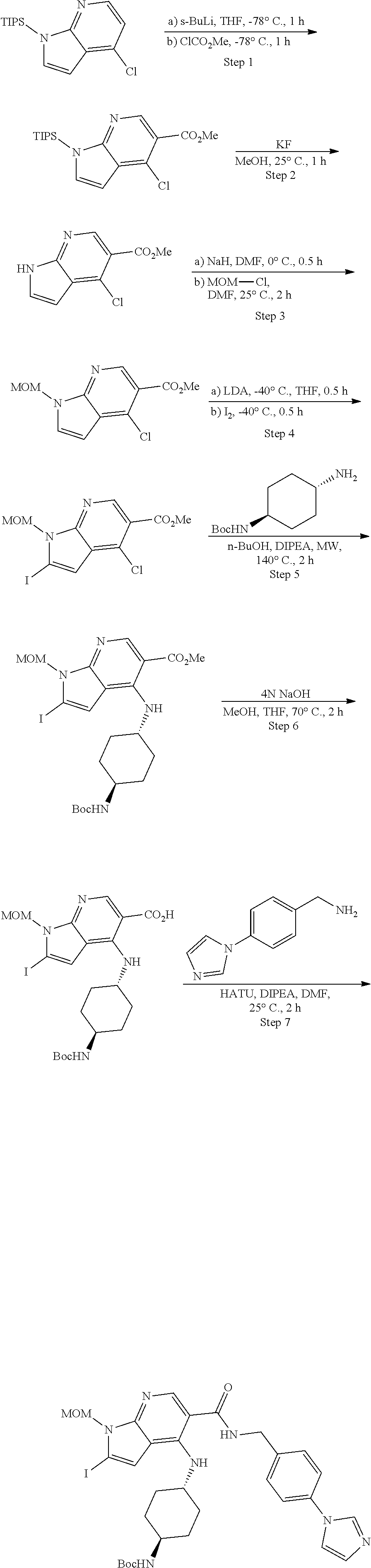

### Step 1: methyl 4-chloro-1-(triisopropylsilyl)-1H-pyrrolo[2,3-b]pyridine-5-carboxylate

A solution of (4-chloropyrrolo[2,3-b]pyridin-1-yl)-triisopropyl-silane (20 g, 64 mmol) in THF (200 mL) was cooled to -78° C. under N₂. sec-BuLi was added (1.3 M, 75 mL) dropwise over 30 minutes under N₂, while the inner temperature was maintained below −60° C. It was stirred at −78° C. for 1 hour. To this mixture was added a solution of methyl carbonochloridate (9.2 g, 97 mmol, 7.5 mL) in THF (50 mL) dropwise at −78° C. over a period of 12 minutes under N₂. The reaction mixture was warmed to room temperature over a period of 1 hour and stirred at room temperature for another one hour. Upon completion, the reaction mixture was quenched by addition of aqueous saturated NH₄Cl (200 mL) at 0° C. and then diluted with water (100 mL). The mixture was extracted with ethyl acetate (3×100 mL). Organic layers were combined, washed with brine (2×50 mL), dried over Na₂SO₄, filtered and concentrated under reduced pressure to give the title compound (26 g, crude) as a red oil, which was used in the next step without further purification.

### Step 2: methyl 4-chloro-1H-pyrrolo[2,3-b]pyridine-5-carboxylate

The synthesis was carried out similarly to Step 3 of Example 1, part B, using the product from the previous step as the starting material.

### Step 3: methyl 4-chloro-1-(methoxymethyl)-1H-pyrrolo[2,3-b]pyridine-5-carboxylate

To a solution of methyl 4-chloro-1H-pyrrolo[2,3-b]pyridine-5-carboxylate (2.3 g, 11 mmol) in dimethylformamide (DMF; 20 mL) was added NaH (0.67 g, 17 mmol, 60% purity) under nitrogen. The mixture was stirred at 0° C. for 0.5 hours. To this was added chloromethyl methyl ether (1.1 g, 13 mmol, 1 mL) and the reaction was stirred at 25° C. for 2 hours. Upon completion, the reaction mixture was quenched by addition aqueous saturated NH₄Cl solution (20 mL) and diluted with water (10 mL). The mixture was extracted with ethyl acetate (3×40 mL). Organic layers were combined, washed with brine (2×25 mL), dried over anhydrous sodium sulfate, filtered and concentrated in vacuo. The residue was purified by column chromatography to give methyl 4-chloro-1-(methoxymethyl)-1H-pyrrolo[2,3-b]pyridine-5-carboxylate (2.3 g, 8.6 mmol, 78% yield) as a yellow solid.

### Step 4: methyl 4-chloro-2-iodo-1-(methoxymethyl)-1H-pyrrolo[2,3-b]pyridine-5-carboxylate

This step was carried out similarly to Step 5 of Example 1, part B, using the product from the previous step as the starting material.

### Step 5: methyl-4-(((1r,4r)-4-((tert-butoxycarbonyl)amino)cyclohexyl)amino)-2-iodo-1-(methoxymethyl)-1H-pyrrolo[2,3-b]pyridine-5-carboxylate

Methyl 4-chloro-2-iodo-1-(methoxymethyl)pyrrolo[2,3-b]pyridine-5-carboxylate (1.6 g, 4 mmol), tert-butyl N-(4-aminocyclohexyl)carbamate (1.4 g, 6.0 mmol) and N,N-diisopropyl ethylamine (DIPEA; 12 mmol, 2.2 mL) were taken up into a microwave tube in n-BuOH (10 mL). The sealed tube was heated at 140° C. for 2 hours via microwave irradiation. The mixture was concentrated in vacuo. The residue was purified by column chromatography to give methyl4-(((1r,4r)-4-((tert-butoxycarbonyl)amino)cyclohexyl)amino)-2-iodo-1-(methoxymethyl)-1H-pyrrolo[2,3-b]pyridine-5-carboxylate (1.3 g, 2 mmol, 51% yield, 93% purity) as a yellow solid.

### Step 6: 4-(((1r,4r)-4-((tert-butoxycarbonyl)amino)cyclohexyl)amino)-2-iodo-1-(methoxymethyl)-1H-pyrrolo[2,3-b]pyridine-5-carboxylic acid

To a solution of methyl 4-[[4-(tert-butoxycarbonylamino)cyclohexyl]amino]-2-iodo-1-(methoxymethyl)pyrrolo[2,3-b]pyridine-5-carboxylate (1.0 g, 1.8 mmol) in THF (5 mL) and MeOH (5 mL) was added NaOH (2 M, 5 mL). It was stirred at 70° C. for 2 hours. The mixture was concentrated under reduced pressure. The residue was dissolved in water (30 mL). The aqueous solution was adjusted pH to 4 with 1 N HCl, then extracted with ethyl acetate (3×60 mL). Organic layers were combined, dried over anhydrous sodium sulfate, filtered and concentrated in vacuo to give the title compound (1.0 g, crude) as a yellow solid.

### Step 7: tert-butyl ((1r,4r)-4-((5-((4-(1H-imidazol-1-yl)benzyl)carbamoyl)-2-iodo-1-(methoxymethyl)-1H-pyrrolo[2,3-b]pyridin-4-yl)amino)cyclohexyl)carbamate

To a solution of 4-[[4-(tert-butoxycarbonylamino)cyclohexyl]amino]-2-iodo-1-(methoxymethyl)pyrrolo[2,3-b]pyridine-5-carboxylic acid (1.4 g, 2.5 mmol) in DMF (10 mL) was added HATU ((dimethylamino)-N,N-dimethyl(3H-[1,2,3]triazolo[4,5-b]yridine-3-yloxy)methaniminium hexafluorophosphate; 1.1 g, 3.0 mmol). The mixture was stirred at 25° C. for 0.5 hour. To this was added (4-imidazol-1-ylphenyl)methanamine (0.48 g, 2.8 mmol) and DIPEA (0.97 g, 7.5 mmol, 1.3 mL). The reaction was stirred at 25° C. for 1.5 hours. Upon completion, the reaction mixture was quenched by addition water (20 mL), and then extracted with ethyl acetate (3×50 mL). Organic layers were combined, washed with brine (2×25 mL), dried over anhydrous sodium sulfate, filtered and concentrated in vacuo. The residue was purified by column chromatography to give the title compound (1.1 g, 1.4 mmol, 57% yield, 91% purity) as a yellow solid.

### F. tert-butyl ((1r,4r)-4-((5-((4-(1H-imidazol-1-yl)benzyl)carbamoyl)-2-iodo-1-((2-(trimethylsilyl)ethoxy)methyl)-1H-pyrrolo[2,3-b]pyridin-4-yl)amino)cyclohexyl) carbamate

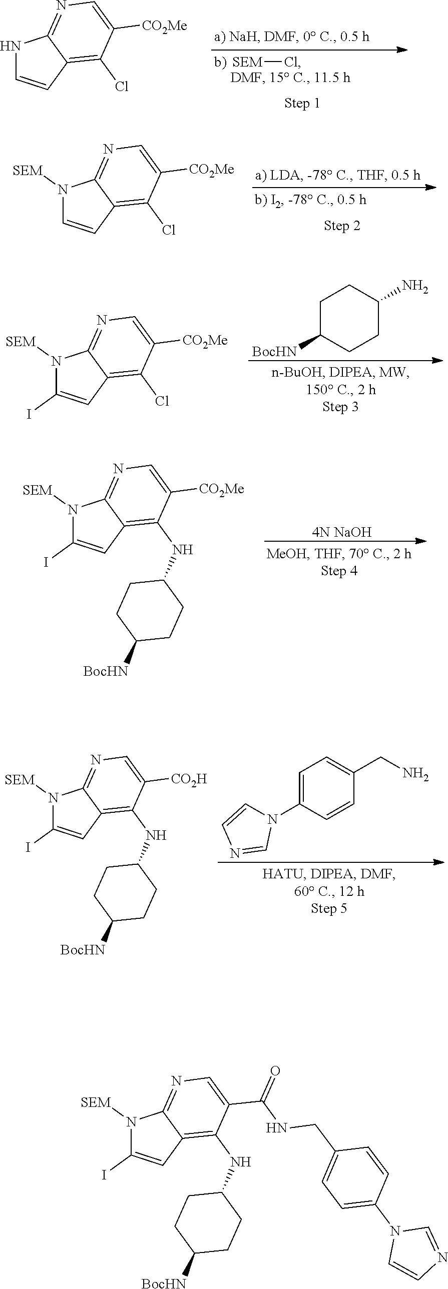

This synthesis was carried out under identical conditions as that described in Steps 3-7 of Example 1, part E, above, except for the use of SEM-C₁ in place of MOM-C₁ in Step 1.

### G. 6-chloro-N-methylpyrimidin-4-amine

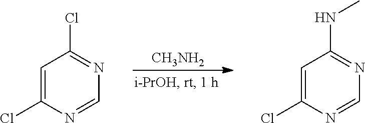

To a suspension of 4,6-dichloropyrimidine (0.50 g, 3.4 mmol) in propan-2-ol (10 mL) was added dropwise methylamine (2 M, 3.4 mL). After addition was completed, the reaction mixture was stirred at room temperature for 1 hour. Upon completion, the reaction mixture was concentrated in vacuo. The residue was dissolved with dichloromethane (50 mL), washed with water (10 mL), brine (2×10 mL) sequentially. The organic phase was collected and concentrated to give 6-chloro-N-methylpyrimidin-4-amine (0.42 g, 86% yield) as a white solid.

### H. 6-chloro-N-isopropylpyrimidin-4-amine

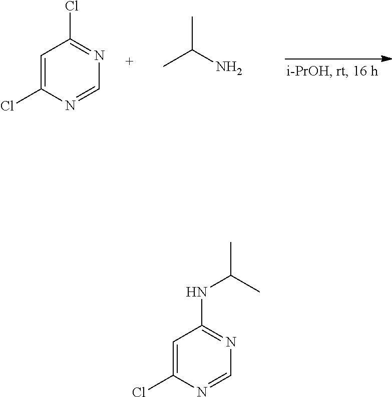

The synthesis was carried out as described above in Example 1, part G.

### I. 4((6-chloropyridin-3-yl)sulfonyl)morpholine

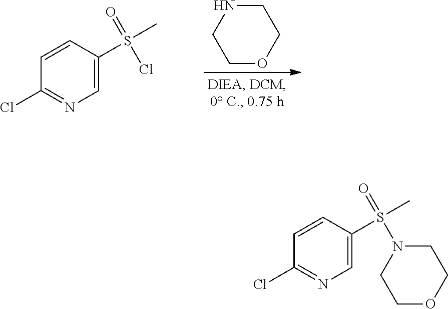

A solution of 6-chloropyridine-3-sulfonyl chloride (500 mg, 2.36 mmol) in dichloromethane (20 mL) was added morpholine (206 mg, 2.36 mmol) at 0° C. The resulting mixture was stirred at 0° C. for 15 min and then DIPEA (458 mg, 3.54 mmol) was added dropwise. The mixture was stirred at 0° C. for 0.5 hour. Upon completion, the mixture was concentrated in vacuo to give the title compound (600 mg, 97% yield) as a white solid.

### J. 1-((3r,5r,7r)-adamantan-1-yl)-N4-((6-bromopyridin-3-yl)methyl) methanamine

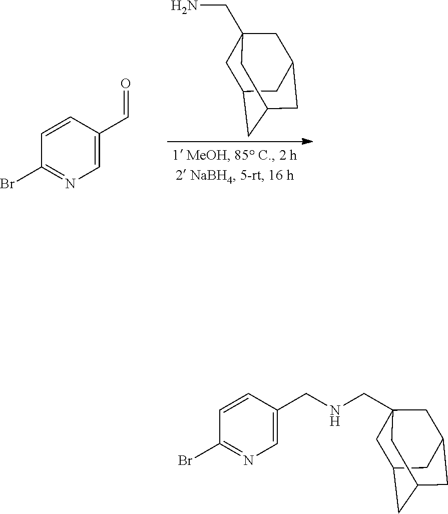 [SMILES: O=Cc1ccc(Br)nc1 ; NCC12CC3CC(CC(C3)C1)C2 ; Brc1ccc(CNCC23CC4CC(CC(C4)C2)C3)cn1]

A mixture of 6-bromopyridine-3-carbaldehyde (300 mg, 1.6 mmol) and 1-adamantylmethanamine (319 mg, 1.9 mmol) in MeOH (5 mL) was heated to 85° C. for 2 hours under nitrogen. Then the mixture was cooled to 5° C. (ice bath), and diluted with more MeOH (5 mL). To this mixture, NaBH₄ (sodium tetrahydroborate; 122 mg, 3.2 mmol) was added. The resulting mixture was slowly allowed to warm up to room temperature and stirred for 16 hours. Upon completion, the mixture was concentrated in vacuo. The residue was quenched by water (30 mL). The mixture was extracted with ethyl acetate (2×50 mL). The organic layer was washed with water (20 mL) and brine (20 mL) sequentially, dried over Na₂SO₄, filtered and concentrated in vacuo. The residue was purified by column chromatography to give 1-((3r,5r,7r)-adamantan-1-yl)-N-((6-bromopyridin-3-yl)methyl) methanamine (500 mg, 93% yield) as a colorless oil.

### K. 2-bromo-5-(pyrrolidin-1-ylmethyl)pyridine

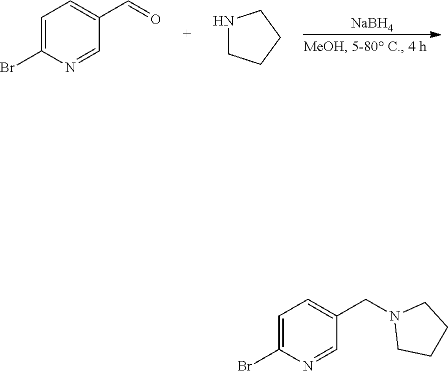 [SMILES: O=Cc1ccc(Br)nc1 ; C1CCNC1 ; "NaBH4" ; Brc1ccc(CN2CCCC2)cn1]

The synthesis was carried out similarly to that described in Example 1, part J, using the above-indicated starting materials.

### L. tert-butyl (1-pyrimidin-2-yl)piperidin-4-yl)carbamate

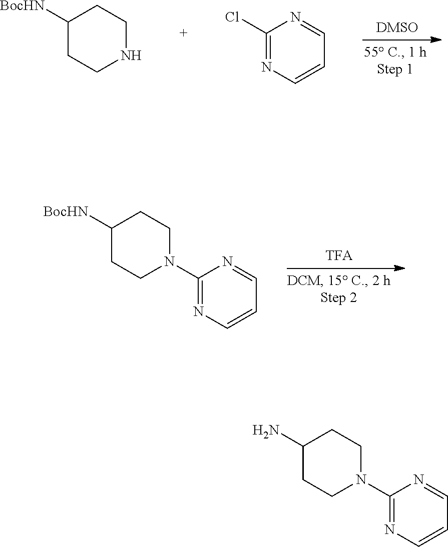 [SMILES: CC(C)(C)OC(=O)NC1CCNCC1 ; Clc1ncccn1 ; CC(C)(C)OC(=O)NC1CCN(c2ncccn2)CC1 ; NC1CCN(c2ncccn2)CC1]

### Step 1: tert-butyl N-(1-pyrimidin-2-yl-4-piperidyl)carbamate

To a mixture of tert-butyl N-(4-piperidyl)carbamate (0.60 g, 3.0 mmol) and 2-chloropyrimidine (0.12 g, 1.0 mmol) in dimethylsulfoxide (DMSO; 1 mL) was stirred at 55° C. for 1 hour. Upon completion, the mixture was poured into aqueous NH₄Cl solution (20 mL, 10%) and filtered to give the title compound (0.8 g, 97% yield) as a white solid.

### Step 2: tert-butyl (1-(pyrimidin-2-yl)piperidin-4-yl)carbamate

To a solution of tert-butyl N-(1-pyrimidin-2-yl-4-piperidyl)carbamate (70 mg, 0.25 mmol) in DCM (2 mL) was added TFA (0.29 g, 2.5 mmol). The mixture was stirred at room temperature for 2 hours. Upon completion, the mixture was concentrated in vacuo and treated with MTBE (methyl tert-butyl ether; 5 mL). The resulting slurry mixture was filtered to give the title compound (36 mg, 52% yield) as a white solid.

### M. (E)-5-bromo-4-chloro-2-(prop-1-en-1-yl)-1H-pyrrolo[2,3-b]pyridine

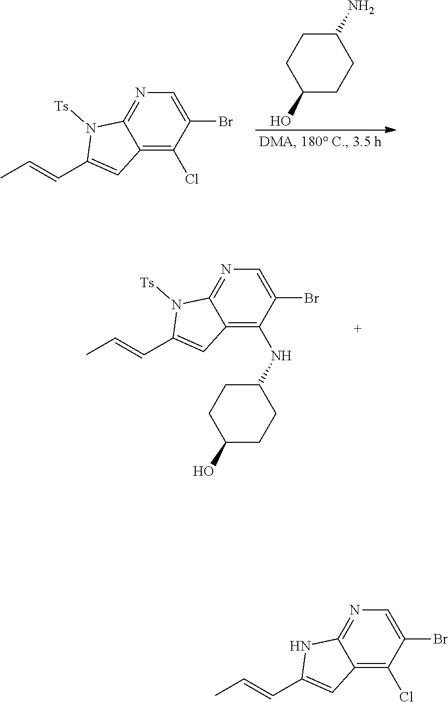

To a mixture of 5-bromo-4-chloro-2-[(E)-prop-1-enyl]-1-(p-tolylsulfonyl)pyrrolo [2,3-b]pyridine (500 mg, 1.17 mmol) and 4-aminocyclohexanol (1.4 g, 11.7 mmol) in a microwave tube was added dimethyl adipate (DMA; 5 mL). The sealed tube was heated at 180° C. for 3.5 hours under nitrogen. Upon completion, the mixture was diluted in ethyl acetate (50 mL), washed with water (20 mL) and brine (20 mL) sequentially. The organic phase was dried over Na₂SO₄, filtered and concentrated in vacuo. The crude was purified by column chromatography to give (1r,4r)-4-((5-bromo-2-((E)-prop-1-en-1-yl)-1-tosyl-1H-pyrrolo[2,3-b]pyridin-4-yl)amino)cyclohexanol (65 mg) as a white solid and (E)-5-bromo-4-chloro-2-(prop-1-en-1-yl)-1H-pyrrolo[2,3-b]pyridine (200 mg, 59% yield) as a white solid.

### N. tert-butyl ((1r,4r)-4-((5-((4-fluorophenyl)carbamoyl)-2-iodo-1-((2-(trimethylsilyl) ethoxy)methyl)-1H-pyrrolo[2,3-b]pyridin-4-yl)amino)cyclohexyl)carbamate

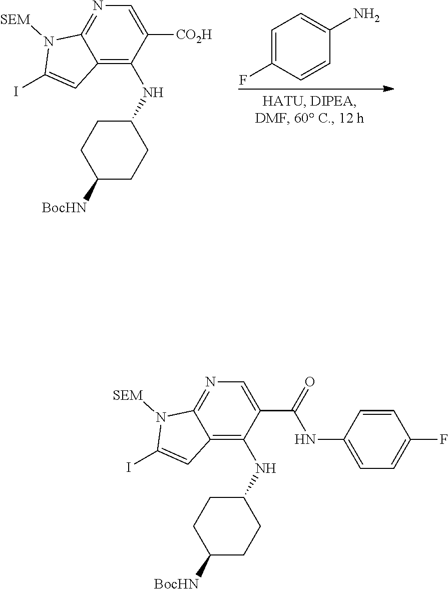 [SMILES: CC(C)(C)OC(=O)N[C@H]1CC[C@H](Nc2c(C(=O)O)cnc3c2cc(I)n3COCC[Si](C)(C)C)CC1 ; Nc1ccc(F)cc1 ; CC(C)(C)OC(=O)N[C@H]1CC[C@H](Nc2c(C(=O)Nc3ccc(F)cc3)cnc3c2cc(I)n3COCC[Si](C)(C)C)CC1]

To a solution of 4-[[4-(tert-butoxycarbonylamino)cyclohexyl]amino]-2-iodo-1-(2-trimethyl silylethoxymethyl)pyrrolo[2,3-b]pyridine-5-carboxylic acid (200 mg) in DMF (3 mL) was added HATU (144 mg). After the mixture was stirred at 60° C. for 0.5 h, 4-fluoroaniline (45 mg) and DIPEA (123 mg) were added. The mixture was stirred at 60° C. for 11.5 h. Upon completion, the reaction mixture was quenched by addition water (50 mL) at 25° C., and then diluted with water (10 mL) and extracted with ethyl acetate (3×30 mL). The combined organic layers were washed with brine (2×25 mL), dried over Na₂SO₄, filtered and concentrated under reduced pressure to give a residue. The residue was purified by prep-TLC to give the title compound (174 mg, 74% yield) as a yellow solid.

### O. 5-bromo-4-chloro-2-ethyl-1-tosyl-1H-pyrrolo[2,3-b]pyridine

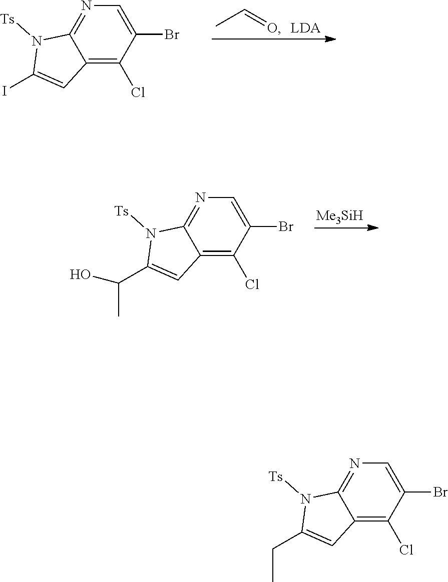

### Step 1: 1-(5-bromo-4-chloro-1-tosyl-1H-pyrrolo[2,3-b]pyridin-2-yl)ethanol

To a solution of 5-bromo-4-chloro-1-(p-tolylsulfonyl)pyrrolo[2,3-b]pyridine (1.0 g, 2.6 mmol) in THF (5 mL) was added LDA (417 mg, 3.9 mmol). The mixture was stirred at −70˜−60° C. for 1 hour, then acetaldehyde (571 mg, 13 mmol) in THF (5 mL) was added to the mixture, the mixture was stirred at −70˜−60° C. for 2 hours. Upon completion, the reaction mixture was quenched by addition saturated NH₄Cl (40 mL) at −70˜−60° C., and then diluted with EA (40 mL) and extracted with EA (3×30 mL). The combined organic layers were washed with brines (2×30 mL), dried over anhydrous Na₂SO₄, filtered and concentrated under reduced pressure to give a residue. The residue was purified by column chromatography to give 1-(5-bromo-4-chloro-1-tosyl-1H-pyrrolo[2,3-b]pyridin-2-yl)ethanol (545 mg, 44% yield).

### Step 2: 5-bromo-4-chloro-2-ethyl-1-tosyl-1H-pyrrolo[2,3-b]pyridine

To a solution of 1-[5-bromo-4-chloro-1-(p-tolylsulfonyl)pyrrolo[2,3-b]pyridin-2-yl]ethanol (400 mg, 0.931 mmol) in DCM (20 mL) was dropwise added triethylsilane (1.6 g, 14 mmol) and diethyl oxonium(trifluoro)boranuide (528 mg, 3.7 mmol) at −78° C., then the mixture was stirred at 40° C. for 16 hours. Upon completion, the reaction mixture was added water 30 mL, and then extracted with DCM (3×20 mL). The combined organic layers were dried over anhydrous Na₂SO₄, filtered and concentrated under reduced pressure to give a residue. The residue was purified by column chromatography to give 5-bromo-4-chloro-2-ethyl-1-tosyl-1H-pyrrolo[2,3-b]pyridine (330 mg, 84% yield).

P. 1-(5-bromo-4-chloro-1-tosyl-1H-pyrrolo[2,3-b]pyridin-2-yl)butan-1-ol

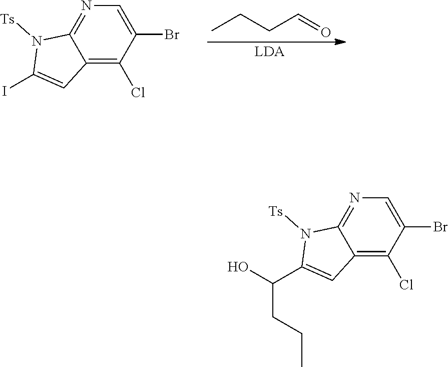

1-(5-bromo-4-chloro-1-tosyl-1H-pyrrolo[2,3-b]pyridin-2-yl)butan-1-ol was synthesized by following the procedure set forth in Step 1 of Example 1, part O.

### Example 2: Synthesis of Compound 108

Compound 108 was synthesized according to General Scheme 1. The step numbers indicated below correspond to the steps shown in that scheme.

### Step 1: 5-bromo-4-chloro-2-propyl-1H-pyrrolo[2,3-b]pyridine

This step was carried out as described in Step 1 of Example 1, part C. (E)-5-bromo-4-chloro-2-(prop-1-en-1-yl)-1H-pyrrolo[2,3-b]pyridine is used as starting material and heated for 6 hours instead of 3 hours to afford the desired product as white solid.

### Step 2: (1r,4r-N1-(5-bromo-2-propyl-1H-pyrrolo[2,3-b]pyridin-4-yl)cyclohexane -1,4-diamine

This step was carried out as described in Step 2 of Example 1, part C, except for heating 14 hours instead of 3.5 hours. This condition affords the desired product as a white solid.

### Step 3: (1r,4r)-N1-(5-(1-methyl-1H-pyrazol-4-yl)-2-propyl-1H-pyrrolo[2,3-b]pyridine-4-yl)cyclohexane-1,4-diamine hydrochloride

A mixture of N4-(5-bromo-2-propyl-1H-pyrrolo[2,3-b]pyridin-4-yl)cyclohexane-1,4-diamine (60 mg, 0.12 mmol), 1-methyl-4-(4,4,5,5-tetramethyl-1,3,2-dioxaborolan-2-yl)pyrazole (48 mg, 0.23 mmol), Pd(dppf)Cl₂ (9 mg, 0.01 mmol) and K₂CO₃ (48 mg, 0.35 mmol) in dioxane (0.5 mL) and H₂O (0.1 mL) was heated to 80° C. for 2 hours under N₂. Upon completion, the mixture was diluted with ethyl acetate (50 mL) and washed with water (20 mL) and brine (20 mL) sequentially. The organic layer dried over Na₂SO₄, filtered and concentrated in vacuo. The residue was purified by prep-HPLC (high pressure liquid chromatography) to afford the product as TFA salt, which was dissolved with water (10 mL), added 0.2 N HCl (0.88 mL), and then lyophilized to give (1r,4r)-N1-(5-(1-methyl-1H-pyrazol-4-yl)-2-propyl-1H-pyrrolo[2,3-b] pyridine-4-yl)cyclohexane-1,4-diamine hydrochloride (13.6 mg) as a white solid. The structure is confirmed by LCMS (liquid chromatography mass spectrometry) and ¹H-NMR (nuclear magnetic resonance).

### Example 3: Synthesis of Compound 106

Compound 106 was synthesized according to General Scheme 1. The step numbers indicated below correspond to the steps shown in that general scheme.

### Step 3: (1r,4r)-4-((5-(4-(morpholinomethyl)phenyl)-2-propyl-1-tosyl-1H-pyrrolo[2,3-b]pyridin-4-yl)amino)cyclohexanol

To a mixture of 4-[[5-bromo-2-propyl-1-(p-tolylsulfonyl)pyrrolo[2,3-b]pyridin-4-yl]amino] cyclohexanol (70 mg, 0.14 mmol), 4-[[4-(4,4,5,5-tetramethyl-1,3,2-dioxaborolan-2-yl)phenyl]methyl]morpholine (126 mg, 0.42 mmol) and K₃PO₄ (88 mg, 0.42 mmol) in DME (1.2 mL) was added [2-(2-aminophenyl)phenyl]-chloro-palladium; dicyclohexyl-[3-(2,4,6-triisopropylphenyl)phenyl]phosphane (11 mg, 0.01 mmol) under N2. The mixture was stirred at 90° C. for 16 hours. Upon completion, the mixture was diluted with ethyl acetate (60 mL), washed with water (20 mL) and brine (20 mL) sequentially. The organic layer was dried over Na₂SO₄, filtered and concentrated in vacuo. The residue was purified by prep-TLC to give (1r,4r)-4-((5-(4-(morpholinomethyl)phenyl)-2-propyl-1-tosyl-1H-pyrrolo[2,3-b] pyridin-4-yl)amino)cyclohexanol (70 mg, 57% yield) as a yellow solid.

### Step 7: (1r,4r)-4-((5-(4-(morpholinomethyl)phenyl)-2-propyl-1H-pyrrolo[2,3-b]pyridin-4-yl)amino)cyclohexanol hydrochloride

To a solution of 4-[[5-[4-(morpholinomethyl)phenyl]-2-propyl-1-(p-tolylsulfonyl) pyrrolo[2,3-b]pyridin-4-yl]amino]cyclohexanol (70 mg, 0.08 mmol) in MeOH (1 mL) and H₂O (1 mL) was added NaOH (16 mg, 0.4 mmol). The mixture was stirred at 90° C. for 12 hours. Upon completion, the mixture was concentrated in vacuum. The solution was concentrated in vacuo. The residue was purified by prep-HPLC to give the title product as TFA salt, which was dissolved with water (10 mL), added 0.2 N HCl (0.622 mL), and then lyophilized to give (1r,4r)-4-((5-(4-(morpholinomethyl)phenyl)-2-propyl-1H-pyrrolo[2,3-b] pyridin-4-yl)amino)cyclohexanol hydrochloride (12.0 mg) as a white solid. The structure is confirmed by LCMS and NMR.

### Example 4. Synthesis of Compound 111

Compound 111 was synthesized according to General Scheme 1. The step numbers indicated below correspond to the steps shown in that general scheme.

### Step 4&5: Tert-butyl ((1r,4r)-4-((5-(6-(methylamino)pyrimidin-4-yl)-2-propyl-1-tosyl-1H-pyrrolo[2,3-b]pyridin-4-yl)amino)cyclohexyl)carbamate

To a solution of tert-butyl ((1r,4r)-4-((5-bromo-2-propyl-1-tosyl-1H-pyrrolo[2,3-b] pyridin-4-yl)amino)cyclohexyl)carbamate (0.15 g, 0.25 mmol) and 4,4,5,5-tetramethyl-2-(4,4,5,5-tetramethyl-1,3,2-dioxaborolan-2-yl)-1,3,2-dioxaborolane (0.19 g, 0.74 mmol) in dioxane (1.50 mL) was added Pd(dppf)Cl₂.CH₂Cl₂ (10 mg, 0.012 mmol) and potassium acetate (73 mg, 0.74 mmol) under nitrogen. The mixture was stirred at 90° C. for 4 hours, then cooled to room temperature. To the reaction mixture was added 6-chloro-N-methylpyrimidin-4-amine (35 mg, 0.25 mmol), potassium carbonate (68 mg, 0.49 mmol), water (0.3 mL) and Pd(PPh₃)₄(terakis(triphenylphosphine) palladium(0); 29 mg, 0.025 mmol). The resulting mixture was degassed and then purged with nitrogen for three times, then stirred at 90° C. for 4 hours. Upon completion, the mixture was poured into water (5 mL), and then extracted with ethyl acetate (2×20 mL). Organic layers were combined, washed with brine (2×5 mL), dried over anhydrous sodium sulfate, filtered and concentrated in vacuo. The residue was purified by prep-TLC to yield tert-butyl ((1r,4r)-4-((5-(6-(methylamino)pyrimidin-4-yl)-2-propyl-1-tosyl-1H -pyrrolo[2,3-b]pyridin-4-yl)amino)cyclohexyl)carbamate (70 mg, 34% purity) as a light white solid.

### Step 7: Synthesis of tert-butyl ((1r,4r)-4-((5-(6-(methylamino)pyrimidin-4-yl)-2-propyl-1H-pyrrolo [2,3-b]pyridin-4-yl)amino)cyclohexyl)carbamate

To a solution of tert-butyl ((1r,4r)-4-((5-(6-(methylamino)pyrimidin-4-yl)-2-propyl -1-tosyl-1H-pyrrolo[2,3-b]pyridin-4-yl)amino)cyclohexyl)carbamate (70 mg, 0.11 mmol) in tetrahydrofuran (1 mL) and methanol (1 mL) was added cesium carbonate (0.11 g, 0.33 mmol) under nitrogen. The mixture was stirred at 70° C. for 12 hours. Upon completion, the mixture was concentrated in vacuo to afford crude product, which was purified by silica gel chromatography to yield tert-butyl ((1r,4r)-4-((5-(6-(methylamino)pyrimidin-4-yl)-2-propyl-1H-pyrrolo [2,3-b]pyridin-4-yl)amino)cyclohexyl)carbamate (20 mg) as a yellow solid.

### Step 8: (1r,4r)-N¹-(5-(6-(methylamino)pyrimidin-4-yl)-2-propyl-1H-pyrrolo[2,3-b]pyridine-4-yl)cyclohexane-1,4-diamine hydrochloride

A solution of tert-butyl ((1r,4r)-4-((5-(6-(methylamino)pyrimidin-4-yl)-2-propyl-1H-pyrrolo [2,3-b]pyridin-4-yl)amino)cyclohexyl)carbamate (20 mg, 0.042 mmol) in dichloromethane (1 mL) and trifluoroacetic acid (1 mL) was stirred at room temperature for 0.5 hour. Upon completion, the mixture was concentrated in vacuo to give crude product, which was purified by prep-HPLC to afford the title compound as TFA salt. The TFA salt was dissolved with water (20 mL), added aqueous HCl solution (0.2 M, 0.30 mL), and then lyophilized to afford (1r,4r-N1-(5-(6-(methylamino)pyrimidin-4-yl)-2-propyl-1H-pyrrolo[2,3-b] pyridine-4-yl)cyclohexane-1,4-diamine hydrochloride (12.0 mg) as a yellow solid. The structure is confirmed by LCMS and NMR.

### Example 5. Synthesis of Compound 118

Compound 118 was synthesized according to General Scheme 1, as depicted above. The step numbers indicated below correspond to the steps shown in that general scheme. Starting material: tert-butyl((1r,4r)-4-((5-(2-chloropyrimidin-4-yl)-2-propyl-1-tosyl-1H-pyrrolo [2,3-b]pyridin-4-yl)amino)cyclohexyl)carbamate

### Steps 4 and 5: tert-butyl((1r,4r)-4-((5-(2-chloropyrimidin-4-yl)-2-propyl-1-tosyl-1H-pyrrolo[2,3-b]pyridin-4-yl)amino)cyclohexyl)carbamate

These steps were carried out in a similar manner to that described in Example 4, steps 4 and 5 using tert-butyl ((1r,4r)-4-((5-bromo-2-propyl-1-tosyl-1H-pyrrolo[2,3-b]pyridin-4-yl)amino) cyclohexyl)carbamate and 2,4-dichloropyrimidine in the reaction to yield the title compound.

### Step 6: tert-butyl ((1r,4r)-4-((2-propyl-5-(2-(pyrrolidin-1-yl)pyrimidin-4-yl)-1-tosyl-1H-pyrrolo[2,3-b]pyridin-4-yl)amino)cyclohexyl)carbamate

To a solution of tert-butyl ((1r,4r)-4-((5-(2-chloropyrimidin-4-yl)-2-propyl-1-tosyl-1H-pyrrolo[2,3-b]pyridin-4-yl)amino)cyclohexyl)carbamate (60 mg, 0.094 mmol) in pyrrolidine (1 mL) was added DIPEA (30 mg, 0.23 mmol). The mixture was stirred at 90° C. for 1 hour. Upon completion, the mixture was concentrated in vacuo to give tert-butyl((1r,4r)-4-((2-propyl-5-(2-(pyrrolidin-1-yl)pyrimidin-4-yl)-1-tosyl-1H-pyrrolo[2,3-b]pyridin-4-yl)amino)cyclohexyl)carbamate (60 mg, crude).

### Step 7: Tert-butyl((1r,4r)-4-((2-propyl-5-(2-(pyrrolidin-1-yl)pyrimidin-4-yl)-1H-pyrrolo [2,3-b]pyridin-4-yl)amino)cyclohexyl)carbamate

Tert-butyl((1r,4r)-4-((2-propyl-5-(2-(pyrrolidin-1-yl)pyrimidin-4-yl)-1-tosyl-1H-pyrrolo [2,3-b]pyridin-4-yl)amino)cyclohexyl)carbamate from the prior step was converted to the title compound by following the procedure set forth in Step 7 of Example 4.

### Step 8: (1r,4r)-N1-(2-propyl-5-(2-(pyrrolidin-1-yl)pyrimidin-4-yl)-1H-pyrrolo[2,3-b]pyridine-4-yl)cyclohexane-1,4-diamine hydrochloride

Tert-butyl((1r,4r)-4-((2-propyl-5-(2-(pyrrolidin-1-yl)pyrimidin-4-yl)-1H-pyrrolo [2,3-b]pyridin-4-yl)amino)cyclohexyl) carbamate from the prior step was converted into the title compound by following the procedure set forth in Step 8 of Example 4.

### Example 6. Synthesis of Compound 100

Compound 100 was synthesized according to general Scheme 2. The step numbers indicated below correspond to the steps shown in that general scheme.

### Step 1: ethyl 4-(((1r,4r)-4-((tert-butoxycarbonyl)amino)cyclohexyl)amino)-2-((E)-prop-1-en-1-yl)-1-tosyl-1H-pyrrolo[2,3-b]pyridine-5-carboxylate

To a solution of tert-butyl((1r,4r)-4-((5-bromo-2-((E)-prop-1-en-1-yl)-1-tosyl-1H-pyrrolo [2,3-b]pyridin-4-yl)amino)cyclohexyl)carbamate (0.24 g, 0.40 mmol) in ethanol (20 mL) was added triethylamine (0.12 g, 1.2 mmol), PdCl₂(PhCN)₂ (38 mg, 991 μmol) and DPPF (1,1′-Bis(diphenylphosphino)ferrocene; 0.13 g, 0.23 mmol) under argon. The suspension was degassed under vacuum, then purged with carbon monoxide for several times. The mixture was stirred under carbon monoxide (3 MPa) at 130° C. for 16 hours. Upon completion, the reaction mixture was filtered and the filtrate was concentrated in vacuo. The crude product was purified by silica gel chromatography to give ethyl 4-(((1r,4r)-4-((tert-butoxycarbonyl)amino)cyclohexyl)amino)-2-((E)-prop-1-en-1-yl) -1-tosyl-1H-pyrrolo[2,3-b]pyridine-5-carboxylate (0.22 g, 77% yield) as a yellow solid.

### Step 2: Ethyl 4-(((1r,4r)-4-((tert-butoxycarbonyl)amino)cyclohexyl)amino)-2-propyl-1-tosyl-1H-pyrrolo[2,3-b]pyridine-5-carboxylate

To a solution of ethyl 4-(((1r,4r)-4-((tert-butoxycarbonyl)amino)cyclohexyl) amino)-2-((E)-prop-1-en-1-yl)-1-tosyl-1H-pyrrolo[2,3-b]pyridine-5-carboxylate (0.17 g, 0.40 mmol) in ethanol (20 mL) was added platinum dioxide (10%, 0.05 g) under nitrogen. The suspension was degassed under vacuum and then purged with hydrogen several times. The mixture was stirred under hydrogen (50 psi) at 40° C. for 16 hours. Upon completion, the reaction mixture was filtered and the filtrate was concentrated in vacuo. The crude product was purified by prep-TLC to afford ethyl 4-(((1r,4r)-4-((tert-butoxycarbonyl)amino)cyclohexyl) amino)-2-propyl-1-tosyl-1H-pyrrolo[2,3-b]pyridine-5-carboxylate (72 mg, 58% yield) as a white solid.

### Step 3: 4-(((1 r,4r)-4-((tert-butoxycarbonyl)amino)cyclohexyl)amino)-2-propyl-1H-pyrrolo[2,3-b]pyridine-5-carboxylic acid

To a solution of ethyl 4-(((1r,4r)-4-((tert-butoxycarbonyl)amino)cyclohexyl)amino) -2-propyl-1-tosyl-1H-pyrrolo[2,3-b]pyridine-5-carboxylate (72 mg, 0.099 mmol) in ethyl alcohol (1 mL) and water (1 mL) was added sodium hydroxide (0.12 g, 3.0 mmol). The mixture was stirred at 90° C. for 16 hours. Upon completion, the mixture was concentrated in vacuo. The residue was diluted with water (20 mL), and the mixture was extracted with ethyl acetate (20 mL). The aqueous layer was adjusted to pH=7 with 4 M acetic acid. The mixture was extracted with ethyl acetate (2×40 mL), washed with water (20 mL), dried over anhydrous sodium sulfate, filtered and concentrated in vacuo to afford 4-(((1r,4r)-4-((tert-butoxycarbonyl)amino)cyclohexyl)amino)-2-propyl-1H-pyrrolo[2,3-b]pyridine-5-carboxylic acid (35 mg, 85% yield) as a white solid.

### Step 4: Tert-butyl ((1r,4r)-4-((5-((4-fluorophenyl)carbamoyl)-2-propyl-1H-pyrrolo[2,3-b]pyridin-4-yl)amino)cyclohexyl)carbamate

To a mixture of 4-(((1r,4r)-4-((tert-butoxycarbonyl)amino)cyclohexyl)amino)-2-propyl-1H-pyrrolo[2,3-b]pyridine-5-carboxylic acid (30 mg, 72 μmol) and 4-fluoroaniline (24 mg, 0.22 mmol) in pyridine (0.4 mL) was added EDCI (1-ethyl-3-(3-dimethyllaminopropyl)-carbodiimide hydrochloride; 28 mg, 0.14 mmol) at room temperature. The mixture was stirred for 6 hours. Upon completion, the mixture was concentrated in vacuo to afford tert-butyl ((1r,4r)-4-((5-((4-fluorophenyl) carbamoyl)-2-propyl-1H-pyrrolo[2,3-b] pyridin-4-yl)amino)cyclohexyl)carbamate (37 mg, crude) as a brown oil.

### Step 5: 4-(((1r,4r)-4-aminocyclohexyl)amino)-N-(4-fluorophenyl)-2-propyl-1H-pyrrolo [2,3-b]pyridine-5-carboxamide hydrochloride

To a solution of tert-butyl ((1r,4r)-4-((5-((4-fluorophenyl)carbamoyl)-2-propyl-1H-pyrrolo [2,3-b]pyridin-4-yl)amino)cyclohexyl)carbamate (37 mg, crude) in dichloromethane (5 mL) was added trifluoroacetic acid (82 mg, 0.72 mmol). The mixture was stirred at room temperature for 2 hours. Upon completion, the mixture was concentrated in vacuo to give crude product. The crude was purified by prep-HPLC to afford the title product as TFA salt, which was dissolved with water (10 mL), added 0.2 N HCl (0.68 mL), and then lyophilized to afford 4-(((1r,4r)-4-aminocyclohexyl)amino)-N-(4-fluorophenyl)-2-propyl-1H-pyrrolo [2,3-b]pyridine-5-carboxamide hydrochloride (6.1 mg) as a white solid. The structure is confirmed by LCMS and NMR.

### Example 7. Synthesis of Compound 119

Compound 119 was synthesized according to General Scheme 3. The step numbers indicated below correspond to the steps shown in that general scheme.

### Step 1: Tert-butyl((1r,4r)-4-((5-((4-(1H-imidazol-1-yl)benzyl)carbamoyl)-1-(methoxymethyl)-2-vinyl-1H-pyrrolo[2,3-b]pyridin-4-yl)amino)cyclohexyl)carbamate

To a solution of tert-butyl ((1r,4r)-4-((5-((4-(1H-imidazol-1-yl)benzyl)carbamoyl)-2-iodo-1-(methoxymethyl)-1H-pyrrolo[2,3-b]pyridin-4-yl)amino)cyclohexyl)carbamate (0.15 g, 0.2 mmol) in t-butyl alcohol (3 mL) and H₂O (0.5 mL) were added 4,4,5,5-tetramethyl-2-vinyl-1,3,2-dioxaborolane (99 mg, 0.6 mmol), [2-(2-aminophenyl)phenyl]-chloro-palladium; tritert-butylphosphane (11 mg, 0.002 mmol) and Cs₂CO₃ (0.21 g, 0.6 mmol). The reaction was stirred at 50° C. for 3 hours under N₂. Upon completion, the solvent was removed and the resulting residue was diluted with ethyl acetate (30 mL). The solution was washed with water (3×15 mL), brine (20 mL) sequentially, filtered and concentrated to give tert-butyl((1r,4r)-4-((5-((4-(1H-imidazol-1-yl)benzyl)carbamoyl)-1-(methoxymethyl)-2-vinyl-1H-pyrrolo[2,3-b]pyridin-4-yl)amino)cyclohexyl)carbamate (0.13 g, crude) as a yellow solid. The crude product was used in the next step without further purification.

### Step 2: Tert-butyl(1r,4r)-4-((5-((4-(1H-imidazol-1-yl)benzyl)carbamoyl)-2-ethyl-1-(methoxymethyl)-1H-pyrrolo[2,3-b]pyridin-4-yl)amino)cyclohexyl)carbamate

To a solution of tert-butyl ((1r,4r)-4-((5-((4-(1H-imidazol-1-yl)benzyl)carbamoyl)-1-(methoxymethyl)-2-vinyl-1H-pyrrolo[2,3-b]pyridin-4-yl)amino)cyclohexyl)carbamate (0.13 g, 0.17 mmol) in MeOH (4 mL) were added Pd/C (37 mg, 0.02 mmol, 5% purity). The mixture was stirred at room temperature for 4 hours under H₂ (15 psi). Upon completion, the reaction slurry was filtered and the filtrate was concentrated to give tert-butyl((1r,4r)-4-((5-((4-(1H-imidazol-1-yl)benzyl)carbamoyl)-2-ethyl-1-(methoxymethyl)-1H-pyrrolo[2,3-b]pyridin-4-yl)amino)cyclohexyl)carbamate (0.12 g, crude) as a dark yellow oil. The crude product was used in the next step without further purification.

### Step 3: N-(4-(1H-imidazol-1-yl)benzyl)-4-(((1r,4r)-4-aminocyclohexyl)amino)-2-ethyl-1H-pyrrolo[2,3-b]pyridine-5-carboxamide

To a solution of tert-butyl N-[4-[[2ethyl-5-[(4-imidazol-1-ylphenyl)methylcarbamoyl]-1-(methoxymethyl)pyrrolo[2,3-b]pyridin-4-yl]amino]cyclohexyl]carbamate (0.12 g, 0.19 mmol) in n-butyl alcohol (1 mL), was added HCl (4 M, 0.4 mL). The mixture was stirred at 140° C. under microwave irradiation for 1 hour. Upon completion, the reaction mixture was filtered and the filtrate was concentrated. The residue was purified by prep-HPLC to give the title product as TFA salt, which was dissolved with water (10 mL), added 1 N HCl (0.5 mL), and then lyophilization to give N-(4-(1H-imidazol-1-yl)benzyl)-4-(((1r,4r)-4-aminocyclohexyl) amino)-2-ethyl-1H-pyrrolo[2,3-b]pyridine-5-carboxamide (23 mg, HCl salt) as a yellow solid. The structure was confirmed by LCMS and ¹HNMR.

### Example 8. Synthesis of Compound 120

Compound 120 was synthesized according to General Scheme 3. The step numbers indicated below correspond to the steps shown in that general scheme.

### Step 1: tert-butyl ((1r,4r)-4-((5-44-(1H-imidazol-1-yl)benzyl)carbamoyl)-2-(cyclopent -1-en-1-yl)-14(2-(trimethylsilyl)ethoxy)methyl)-1H-pyrrolo[2,3-b]pyridin-4-yl)amino) cyclohexyl)carbamate

Tert-butyl N-[4-[[5-[(4-imidazol-1-ylphenyl)methylcarbamoyl]-2-iodo-1-(2-trimethylsilylethoxymethyppyrrolo[2,3-b]pyridin-4-yl]amino]cyclohexyl]carbamate (0.10 g, 0.13 mmol), 2-(cyclopenten-1-yl)-4,4,5,5-tetramethyl-1,3,2-dioxaborolane (30 mg, 0.15 mmol), K₃PO₄ (1.5 M, 0.25 mL) and Xphos-Pd-G3 (9 mg, 0.01 mmol) in DMA (3 mL) were degassed and then heated to 60° C. for 12 hours under N₂. The reaction mixture was quenched by addition water (10 mL) at room temperature, and then diluted with water (20 mL). The mixture was extracted with ethyl acetate (3 ×20 mL). Organic layers were combined, washed with brine (30 mL), dried over Na₂SO₄, filtered and concentrated in vacuo to give tert-butyl ((1r,4r)-4-((5-((4-(1H-imidazol-1-yl)benzyl)carbamoyl)-2-(cyclopent-1-en-1-yl)-1-((2-(trimethylsilypethoxy)methyl)-1H-pyrrolo[2,3-b]pyridin-4-yl)amino)cyclohexyl)carbamate (110 mg, crude) as a yellow solid.

### Step 2: Tert-butyl ((1r,4r)-4-((5-((4-(1H-imidazol-1-yl)benzyl)carbamoyl)-2-cyclopentyl-1-((2-(trimethylsilyl)ethoxy)methyl)-1H-pyrrolo[2,3-b]pyridin-4-yl) amino)cyclohexyl)carbamate

The title product was synthesized from the product of the previous step as described in step 2 of Example 7, except the reaction was heated for 18 hours instead of 4 hours.

### Step 4: N-(4-(1H-imidazol-1-yl)benzyl)-4-(((1r,4r)-4-aminocyclohexyl)amino)-2-cyclopentyl-1H-pyrrolo [2,3-b]pyridine-5-carboxamide

A solution of tert-butyl N-[4-[[2-cyclopentyl-5-[(4-imidazol-1-ylphenyl)methylcarbamoyl]-1-(2-trimethylsilylethoxymethyl)pyrrolo[2,3-b]pyridin-4-yl]amino]cyclohexyl]carbamate (80 mg, 0.11 mmol) in DCM (1 mL) was added TFA (1 mL, 14 mmol). The reaction was stirred at room temperature for 1 hour. The volatile was removed and the residue was added MeOH (2 mL) and ethane-1,2-diamine (0.45 g, 7 mmol, 0.5 mL). The mixture was stirred at 50° C. for 1 hour. The mixture was concentrated in vacuo, then purified by prep-HPLC. Fractions were collected, concentrated and lyophilized to give the title compound (7 mg) as a white solid. The structure was confirmed by LCMS and NMR.

### Example 9. Synthesis of Compound 128

Compound 128 was synthesized according to General Scheme 3. The step numbers indicated below correspond to the steps shown in that general scheme.

### Step 1: tert-butyl N-[4-[[5[(4-fluorophenyl)carbamoyl]-1-(2-trimethylsilylethoxymethyl)-2-(2-trimethylsilylethynyl)pyrrolo[2,3-b]pyridin-4-yl]amino]cyclohexyl]carbamate

A mixture of tert-butyl N-[4-[[5-[(4-fluorophenyl)carbamoyl]-2-iodo-1-(2-t rimethylsilylethoxymethyl)pyrrolo[2,3-b]pyridin-4-yl]amino]cyclohexyl]carbamate (176 mg), ethynyl(trimethyl)silane (72 mg, 0.1 mL), TEA (triethylamine; 0.1 mL), CuI (9 mg) and Pd(PPh₃)₂Cl₂ (17 mg) in THF (5 mL) was degassed and purged with N₂ for three times, and then the mixture was stirred at room temperature for 4 hours under N₂ atmosphere. Upon completion, the mixture was filtered through celite to remove catalyst and inorganic salts. The filtrate was concentrated in vacuo and the resulting residue was purified by prep-TLC to give the title compound (140 mg) as a yellow solid.

### Step 4: [4-1(4-aminocyclohexyl)amino]-2-ethynyl-N-(4-fluorophenyl)-1H-pyrrolo[2,3-b]pyridine-5-carboxamide

A solution of tert-butyl N-[4-[[5-[(4-fluorophenyl)carbamoyl]-1-(2-trimethylsilylethoxymethyl)-2-(2-trimethylsilylethynyl)pyrrolo[2,3-b]pyridin-4-yl]amino]cyclohexyl]carbamate (135 mg) in DCM (2 mL) was added TFA (trifluoroacetic acid; 2 mL). The mixture was stirred at room temperature for 12 hours. The mixture was concentrated, then treated with MeOH (2 mL) and ethane-1,2-diamine (0.2 mL). After the reaction was stirred at room temperature for 12 hours, K₂CO₃ (20 mg) was added and the reaction was stirred for another 1 hour. Upon completion, the mixture was concentrated under reduced pressure to give a residue. The residue was purified by prep-HPLC. After prep-HPLC, the mixture was concentrated in vacuo to remove organic solvent, then lyophilized to give the title compound (13 mg) as a light yellow solid. The structure was confirmed by LCMS and ¹H NMR.

### Example 10. Synthesis of Compound SY-4475

Compound SY-4475 was synthesized according to General Scheme 4. The step numbers indicated below correspond to the steps shown in that general scheme.

### Step 1: methyl 4-chloro-2-(2-hydroxybutan-2-yl)-1-((2-(trimethylsilyl)ethoxy)methyl)-pyrrolo[2,3-b]pyridine-5-carboxylate

To a solution of methyl 4-chloro-1-(2-trimethylsilylethoxymethyl)pyrrolo[2,3-b]pyridine-5-carboxylate (2.0 g, 5.9 mmol) in THF (tetrahydrofuran; 50 mL) was added LDA (lithium diisopropylamide; 2 M, 4.1 mL) at −78° C. under N₂. It was stirred at −78° C. for 0.5 hour. After the mixture was added butan-2-one (0.63 mL, 7.0 mmol) in THF (50 mL) at −78° C. The mixture was stirred at −78° C. for another 0.5 hour. Upon completion, the reaction was quenched by addition of sat. NH₄Cl (2 mL). The reaction mixture was concentrated in vacuo and the resulting residue was purified by silica gel column chromatography to afford the title compound (1.63 g) as light yellow oil.

### Step 2: methyl 4-(((1r,4r)-4-((tert-butoxycarbonyl)amino)cyclohexyl)amino)-2-(2-hydroxybutan-2-yl)-1-((2-(trimethylsilyl)ethoxy)methyl)-1H-pyrrolo[2,3-b]pyridine-5-carboxylate

Methyl 4-chloro-2-(1-hydroxy-1-methyl-propyl)-1-(2-trimethylsilylethoxymethyl) pyrrolo[2,3-b]pyridine-5-carboxylate (413 mg, 1.0 mmol), tert-butyl N-(4-aminocyclohexyl)carbamate (257 mg, 1.2 mmol) and DIPEA (0.523 mL, 3.0 mmol) were taken up into a microwave tube in n-BuOH (4.0 mL). The sealed tube was heated at 140° C. for 2 hrs under microwave irradiation. Upon completion, the reaction mixture was concentrated in vacuo and the resulting residue was diluted with water (60 mL) and extracted with dichloromethane (150 mL). The organic layer was washed with brine (60 mL), dried over Na₂SO₄, filtered and concentrated in vacuo to give a residue. The residue was purified by silica gel column chromatography to afford the title compound (270 mg, 46% yield) as a yellow solid.

### Step 3: 4-(((1r,4r)-4-((tert-butoxycarbonyl)amino)cyclohexyl)amino)-2-(2-hydroxybutan-2-yl)-1-((2-(trimethylsilyl)ethoxy)methyl)-1H-pyrrolo[2,3-b]pyridine-5-carboxylic acid

The title product was synthesized as described in Step 6 of Example 1, part E, above, from the product of the previous step.

### Step 4: tert-butyl ((1r,4r)-4-((5-((4-fluorophenyl)carbamoyl)-2-(2-hydroxybutan-2-yl)-1-((2-(trimethylsilyl) ethoxy)methyl)-1H-pyrrolo[2,3-b]pyridin-4-yl)amino)cyclohexyl)carbamate

The title product was synthesized as described in Example 1, part N, from the product of the previous step.

### Step 5: 4-(((1r,4r)-4-aminocyclohexyl)amino)-2-(E)-but-2-en-2-yl)-N-(4-fluorophenyl)-1H-pyrrolo[2,3-b]pyridine-5-carboxamide

The solution of tert-butyl N-[4-[[5-[(4-fluorophenyl)carbamoyl]-2-(1-hydroxy-1-methylpropyl)-1-(2-trimethylsilylethoxymethyl)pyrrolo[2,3-b]pyridin-4-yl]amino]cyclohexyl]carbamate (120 mg, 0.179 mmol) in DCM (2 mL) was added TFA (1.0 mL, 13.5 mmol). Then the mixture was stirred at room temperature for 2 hours. Upon completion, the mixture was concentrated in vacuum and the solution of the residue in MeOH (methanol; 2 mL) was added ethane-1,2-diamine (1.0 mL, 9.6 mmol). Then the mixture was stirred at room temperature for 12 hours. The reaction mixture was concentrated under reduced pressure to afford the title compound (70 mg, crude) as a yellow solid.

### Step 6: 4-(((1r,4r)-4-aminocyclohexyl)amino)-2-(sec-butyl)-N-(4-fluorophenyl)-1H-pyrrolo[2,3-b]pyridine-5-carboxamide

To a solution of 4-[(4-aminocyclohexyl)amino]-N-(4-fluorophenyl)-2-[(E)-1-methylprop-1-enyl]-1H-pyrrolo[2,3-b]pyridine-5-carboxamide (70 mg, 0.166 mmol) and NH₃.H₂O (1 mL, 6.5 mmol, 25% purity) in MeOH (5 mL) was added Pd/C (35 mg, 0.017 mmol) under N₂. The suspension was degassed under vacuum and purged with H₂ several times. The mixture was stirred under H₂ (15 psi) at 25° C. for 36 hours. Upon completion, the reaction mixture was filtered through a pad of celite. The residue was purified by prep-HPLC. After prep-HPLC purification, the eluent was concentrated to remove organic solvent. The residual aqueous solution was lyophilized to give the title compound (17.0 mg, 24% yield) was obtained as a white solid. The structure was confirmed by LCMS and NMR.

### Example 11. Synthesis of Compound 135

Compound 135 was synthesized according to General Scheme 3. The step numbers indicated below correspond to the steps shown in that general scheme.

### Step 1. tert-butyl ((1r,4r)-4-((5-((4-fluorophenyl)carbamoyl)-2-(trifluoromethyl)-1-((2-(trimethylsilyl)ethoxy)methyl)-1H-pyrrolo[2,3-b]pyridin-4-yl)amino)cyclohexyl) carbamate

A solution of tert-butyl N-[4-[[5-[(4-fluorophenyl)carbamoyl]-2-iodo-1-(24 rimethylsilylethoxymethyl)pyrrolo[2,3-b]pyridin-4-yl]amino]cyclohexyl]carbamate (100 mg, 0.138 mmol), methyl 2,2-difluoro-2-fluorosulfonyl-acetate (0.053 mL, 0.415 mmol) and CuI (53 mg, 0.276 mmol) in DMF (5 mL) was stirred at 80° C. for 15 hours. Upon completion, the reaction mixture was concentrated under reduced pressure to remove solvent. The residue was purified by prep-TLC (thin layer chromatography) to afford the title compound (45 mg, crude) as a light-yellow solid.

### Step 4: 4-(((1r,4r)-4-aminocyclohexyl)amino)-N-(4-fluorophenyl)-2-(trifluoromethyl)-1H-pyrrolo[2,3-b]pyridine-5-carboxamide hydrochloride

This step was carried out as described in Step 4 of Example 8, from the product of the prior step. This condition affords the desired product as a yellow solid. The structure was confirmed by LCMS and ¹H NMR.

### Example 12. Synthesis of Compound 138

Compound 138 was synthesized according to General Scheme 3. The step numbers indicated below correspond to the steps shown in that general scheme.

### Step 1. tert-butyl((1r,4r)-4-((2-cyclobutyl-5-((4-fluorophenyl)carbamoyl)-1-((2-(trimethylsilyl)ethoxy)methyl)-1H-pyrrolo[2,3-b]pyridin-4-yl)amino) cyclohexyl)carbamate

A mixture of tert-butyl N-[4-[[5-[(4-fluorophenyl)carbamoyl]-2-iodo-1-(2-trimethylsilyl ethoxymethyl)pyrrolo[2,3-b]pyridin-4-yl]amino]cyclohexyl]carbamate (0.13 g, 0.16 mmol), bromocyclobutane (0.073 mL, 0.78 mmol), tris(trimethylsilyl)silane (0.24 mL 0.78 mmol), dichloronicke1;1,2-dimethoxyethane (1.7 mg, 0.0078 mmol), 4-tert-butyl-2-(4-tert-butyl-2-pyridyl)pyridine (2.1 mg, 0.0078 mmol), bis[2-(2-pyridyl)phenyl]iridium(1+);4-tert-butyl-2-(4-tert-butyl-2-pyridyl)pyridine;hexafluorophosphate (2.8 mg, 0.0031 mmol) and sodium carbonate (33 mg, 0.32 mmol) in dimethoxyethane (2 mL) was stirred under nitrogen atmosphere and blue light irradiation for 16 hours at 50° C. Upon completion, the reaction mixture was diluted with ethyl acetate (20 mL). The mixture was washed by brine (2×10 mL). The organic lay was dried, filtered and concentrated to dryness. The residue was purified by column chromatography as an impurity product. The product was further purified by prep-TLC to give the title compound (10 mg) as a yellow solid.

### Step 4: 4-(((1r,4r)-4-aminocyclohexyl)amino)-2-cyclobutyl-N-(4-fluorophenyl)-1H-pyrrolo[2,3-b]pyridine-5-carboxamide hydrochloride

This step was carried out as described in Step 4 of Example 8, from the product of the prior step. This condition affords the desired product as a white solid. The structure was confirmed by LCMS and ¹H NMR.

The synthetic protocols and intermediates described above were used to prepare other compounds disclosed herein as indicated in Table 2, below. The NMR and LC-MS data obtained for these compounds are shown below. Those of skill in the art will be readily able to make other compounds of the invention based on the general synthesis schemes, intermediate synthesis protocols and specific compound synthesis protocols set forth herein.

TABLE 2
Synthesis and Physicochemical Properties of Exemplary Compounds.

| Compound | Synthetic | | MS |
|---|---|---|---|
| Number | Protocol | ¹H NMR | (M + 1) |
| 100 | 2 | ¹H NMR (400 MHz, CD₃OD) δ 8.43 (s, 1H), 7.65-7.62 (m, | 410.1 |
| | | 2H), 7.15-7.10 (m, 2H), 6.56 (s, 1H), 4.15 (m, 1H), 3.24-3.21 | |
| | | (m, 1H), 2.79 (t, J = 7.2 Hz, 2H), 2.35-2.32 (m, 1H), 2.19- | |
| | | 2.16 (m, 2H), 1.84-1.57 (m, 6H), 1.04 (t, J = 7.2 Hz, 3H). | |
| 101 | 2 | ¹H NMR (400 MHz, CD₃OD) δ 8.40 (s, 1H), 7.65-7.61 (m, | 411.3 |
| | | 2H), 7.14-7.10 (m, 2H), 6.56 (s, 1H), 4.12 (m, 1H), 3.71-3.70 | |
| | | (m, 1H), 2.78 (t, J = 7.6 Hz, 2H), 2.21 (m, 2H), 2.04 (m, 2H), | |
| | | 1.83-1.74 (m, 2H), 1.61-1.53 (m, 4H), 1.04 (t, J = 7.2 Hz, 3H). | |
| 102 | 2 | ¹H NMR (400 MHz, CD₃OD) δ 9.49 (s, 1H), 9.22 (s, 1H), | 473.2 |
| | | 8.35 (s, 1H), 8.09 (t, J = 2.0 Hz, 1H), 7.80-7.66 (m, 5H), 6.55 | |
| | | (s, 1H), 4.66-4.65 (m, 1H), 4.11 (m, 1H), 3.70 (m, 1H), 2.78 | |
| | | (t, J = 7.6 Hz, 2H), 2.22 (m, 2H), 2.02 (m, 2H), 1.83-1.73 (m, | |
| | | 2H), 1.54-1.52 (m, 4H), 1.03 (t, J = 7.6 Hz, 3H). | |
| 103 | 2 | ¹H NMR (400 MHz, CD₃OD) δ 8.45 (s, 1H), 7.67-7.63 (m, | 382.1 |
| | | 2H), 7.14-7.09 (m, 2H), 6.59 (s, 1H), 4.18-4.09 (m, 1H), | |
| | | 3.26-3.21 (m, 1H), 2.48 (s, 3H), 2.34-2.31 (m, 2H), 2.18- | |
| | | 2.16 (m, 2H), 1.78-1.69 (m, 2H), 1.65-1.56 (m, 2H). | |
| 104 | 2 | ¹H NMR (400 MHz, CD₃OD) δ 8.61 (d, J = 5.2 Hz, 2H), 8.33 | 478.3 |
| | | (s, 1H), 7.00 (t, J = 5.2 Hz, 1H), 6.52 (s, 1H), 4.62 (d, J = 13.6 | |
| | | Hz, 2H), 4.30-4.24 (m, 1H), 4.10 (m, 1H), 3.72-3.70 (m, 1H), | |
| | | 3.45 (t, J = 12.4 Hz, 2H), 2.76 (t, J = 7.6 Hz, 2H), 2.22-2.17 | |
| | | (m, 4H), 2.05-2.02 (m, 2H), 1.82-1.72 (m, 4H), 1.59-1.52 | |
| | | (m, 4H), 1.04 (t, J = 7.6 Hz, 3H). | |
| 105 | 1 | ¹H NMR (400 MHz, CD₃OD) δ 8.60 (d, J = 2.0 Hz, 1H), 8.28 | 450.2 |
| | | (s, 1H), 7.95-7.93 (m, 1H), 7.85 (d, J = 8.0 Hz, 1H), 6.49 (s, | |
| | | 1H), 4.13-4.11 (m, 1H), 3.74-3.68 (m, 7H), 2.76 (t, J = 7.6 | |
| | | Hz, 2H), 2.60-2.54 (m, 4H), 2.25-2.20 (m, 2H), 2.05-2.01 (m, | |
| | | 2H), 1.83-1.73 (m, 2H), 1.60-1.50 (m, 4H), 1.04 (t, J = 7.6 | |
| | | Hz, 3H). | |
| 106 | 1 | ¹H NMR (400 MHz, CD₃OD) δ 7.77 (d, J = 8.0 Hz, 2H), 7.63 | 449.3 |
| | | (s, 1H), 7.58 (d, J = 8.0 Hz, 2H), 6.54 (s, 1H), 4.49 (s, 2H), | |
| | | 4.08-4.02 (m, 3H), 3.87 (t, J = 12.0 Hz, 3H), 3.57-3.56 (m, | |
| | | 1H), 3.46 (d, J = 12.4 Hz, 2H), 3.26 (d, J = 11.6 Hz, 2H), 2.80 | |
| | | (t, J = 7.6 Hz, 2H), 2.07-2.05 (m, 2H), 1.98-1.95 (m, 2H), | |
| | | 1.85-1.75 (m, 2H), 1.50-1.44 (m, 4H), 1.04 (t, J = 7.6 Hz, 3H). | |
| 107 | 1 | ¹H NMR (400 MHz, CD₃OD) δ 8.01 (s, 1H), 7.72 (s, 1H), | 411.3 |
| | | 7.64 (s, 1H), 6.49 (s, 1H), 4.69 (t, J = 5.6 Hz, 2H), 4.10-4.05 | |
| | | (m, 1H), 3.76 (t, J = 5.6 Hz, 2H), 3.66-3.60 (m, 1H), 3.01 | |
| | | (s, 6H), 2.78 (t, J = 7.6 Hz, 2H), 2.12-2.09 (m, 2H), 2.03-1.99 | |
| | | (m, 2H), 1.82-1.74 (m, 2H), 1.66-1.57 (m, 2H), 1.52-1.44 | |
| | | (m, 2H), 1.04 (t, J = 7.2 Hz, 3H). | |
| 108 | 1 | ¹H NMR (400 MHz, CD₃OD) δ 8.02 (s, 1H), 7.82 (s, 1H), | 353.3 |
| | | 7.69 (s, 1H), 6.54 (s, 1H), 4.20-4.10 (m, 1H), 4.05 (s, 3H), | |
| | | 3.20-3.10 (m, 1H), 2.79 (t, J = 7.6 Hz, 2H), 2.23-2.10 (m, | |
| | | 4H), 1.84-1.56 (m, 6H), 1.03 (t, J = 7.2 Hz, 3H). | |
| 109 | 1 | ¹H NMR (400 MHz, CD₃OD) δ 8.88 (s, 1H), 8.42 (s, 1H), | 528.3 |
| | | 8.27 (d, J = 7.2 Hz, 1H), 8.08 (d, J = 7.6 Hz, 1H), 6.59 (s, | |
| | | 1H), 4.41 (s, 2H), 4.22-4.20 (m, 1H), 3.80-3.70 (m, 1H), | |
| | | 2.83-2.79 (m, 4H), 2.30-2.26 (m, 2H), 2.10-2.05 (m, 5H), | |
| | | 1.84-1.60 (m, 18H), 1.06 (t, J = 7.6 Hz, 3H). | |
| 110 | 3 | ¹H NMR (400 MHz, CD₃OD) δ 8.33 (s, 1H), 8.14 (s, 1H), | 506.3 |
| | | 7.84-7.77 (m, 2H), 7.61-7.52 (m, 5H), 7.46 (t, J = 7.6 Hz, | |
| | | 2H), 7.38-7.30 (m, 1H), 7.16 (s, 1H), 6.93 (s, 1H), 4.61 (s, | |
| | | 2H), 4.10-4.05 (m, 1H), 2.98-2.87 (m, 1H), 2.32-2.30 (m, | |
| | | 2H), 2.08-2.06 (m, 2H), 1.41-1.03 (m, 4H). | |
| 111 | 1 | ¹H NMR (400 MHz, D₂O) δ 8.59 (s, 1H), 7.86 (s, 1H), 6.88 | 380.2 |
| | | (s, 1H), 6.48 (s, 1H), 4.14-4.09 (m, 1H), 3.22-3.17 (m, 1H), | |
| | | 3.03 (s, 3H), 2.71 (t, J = 7.2 Hz, 2H), 2.21-2.08 (m, 4H), 1.71- | |
| | | 1.44 (m, 6H), 0.88 (t, J = 7.2 Hz, 3H). | |
| 112 | 1 | ¹H NMR (400 MHz, D₂O) δ 8.56 (s, 1H), 7.84 (s, 1H), 6.78 | 408.2 |
| | | (s, 1H), 6.49 (s, 1H), 4.35-4.25 (m, 1H), 4.14-4.09 (m, 1H), | |
| | | 3.22-3.17 (m, 1H), 2.71 (t, J = 7.2 Hz, 2H), 2.21-2.28 (m, | |
| | | 2H), 2.11-2.08 (m, 2H), 1.72-1.63 (m, 2H), 1.63-1.43 | |
| | | (m, 4H), 1.24 (d, J = 6.8 Hz, 6H), 0.88 (t, J = 7.2 Hz, 3H). | |
| 113 | 1 | ¹H NMR (400 MHz, D₂O) δ 8.84 (d, J = 4.8 Hz, 1H), 8.40 | 350.2 |
| | | (dd, J₁ = 8.0 Hz, J₂ = 6.8 Hz, 1H), 8.21 (s, 1H), 8.13 (d, J = | |
| | | 8.0 Hz, 1H), 7.86-7.83 (m, 1H), 6.61 (s, 1H), 4.20-4.15 (m, | |
| | | 1H), 3.24-3.18 (m, 1H), 2.82 (t, J = 7.6 Hz, 2H), 2.32-2.29 | |
| | | (m, 2H), 2.19-2.16 (m, 2H), 1.85-1.59 (m, 6H), 1.05 (t, J = | |
| | | 7.6 Hz, 3H). | |
| 114 | 1 | ¹H NMR (400 MHz, D₂O) δ 8.60 (d, J = 2.8 Hz, 1H), 8.29 | 368.2 |
| | | (s, 1H), 7.98 (dd, J1 = 8.8 Hz, J2 = 4.0 Hz, 1H), 7.85-7.80 | |
| | | (m, 1H), 6.57 (s, 1H), 4.20-4.15 (m, 1H), 3.29-3.23 (m, 1H), | |
| | | 2.80 (t, J = 7.6 Hz, 2H), 2.36-2.33 (m, 2H), 2.21-2.18 (m, | |
| | | 2H), 1.84-1.60 (m, 6H), 1.04 (t, J = 7.6 Hz, 3H). | |
| 115 | 1 | ¹H NMR (400 MHz, CD₃OD) δ 9.02 (d, J = 2.0 Hz, 1H), 8.51 | 499.3 |
| | | (s, 1H), 8.29 (dd, J₁ = 8.4 Hz, J₂ = 2.4 Hz, 1H), 8.19 (d, J = | |
| | | 9.2 Hz, 1H), 6.60 (s, 1H), 4.24-4.20 (m, 1H), 3.76 (t, J = 4.8 | |
| | | Hz, 4H), 3.27-3.25 (m, 1H), 3.70 (t, J = 4.8 Hz, 4H), 2.81 (t, | |
| | | J = 7.6 Hz, 2H), 2.40-2.35 (m, 2H), 2.25-2.29 (m, 2H), 1.85- | |
| | | 1.69 (m, 6H), 1.05 (t, J = 7.6 Hz, 3H). | |
| 116 | 1 | ¹H NMR (400 MHz, D₂O) δ 8.57 (s, 1H), 7.88 (s, 1H), 6.92 | 366.1 |
| | | (s, 1H), 6.48 (s, 1H), 4.18-4.04 (m, 1H), 3.26-3.13 (m, | |
| | | 1H), 2.70 (t, J = 7.2 Hz, 2H), 2.21-2.28 (m, 2H), 2.11-2.08 | |
| | | (m, 2H), 1.71-1.63 (m, 2H), 1.62-1.43 (m, 4H), 0.88 (t, | |
| | | J = 7.2 Hz, 3H). | |
| 117 | 1 | ¹H NMR (400 MHz, D₂O) δ 8.06 (s, 1H), 7.77 (d, J = 3.2 Hz, | 356.1 |
| | | 1H), 7.48 (d, J = 3.6 Hz, 1H), 6.33 (s, 1H), 3.95-3.92 (m, 1H), | |
| | | 3.24-3.19 (m, 1H), 2.64 (t, J = 7.2 Hz, 2H), 2.22-2.02 (m, | |
| | | 4H), 1.70-1.40 (m, 6H), 0.88 (t, J = 7.2 Hz, 3H). | |
| 118 | 1 | ¹H NMR (400 MHz, D₂O) δ 8.40 (s, 1H), 8.09 (d, J = 6.8 Hz, | 420.2 |
| | | 1H), 7.13 (d, J = 6.8 Hz, 1H), 6.46 (s, 1H), 4.18-4.13 (m, 1H), | |
| | | 3.70-3.52 (m, 4H), 3.26-3.20 (m, 1H), 2.68 (t, J = 7.2 Hz, | |
| | | 2H), 2.30-2.27 (m, 2H), 2.14-2.07 (m, 6H), 1.69-1.56 (m, | |
| | | 4H), 1.55-1.43 (m, 2H), 0.87 (t, J = 7.2 Hz, 3H). | |
| 119 | 3 | ¹H NMR (400 MHz, CD₃OD) δ 9.46 (s, 1H), 8.38 (s, 1H), | 458.2 |
| | | 8.08 (s, 1H), 7.84-7.82 (m, 1H), 7.82-7.61 (m, 4H), 6.53 (s, | |
| | | 1H), 4.66 (s, 2H), 4.22-4.07 (m, 1H), 3.25 (t, J = 11.2 Hz, 1H), | |
| | | 2.84 (q, J = 7.6 Hz, 2H), 2.34 (d, J = 12.8 Hz, 2H), 2.19 (d, | |
| | | J = 11.6 Hz, 2H), 1.78-1.55 (m, 4H), 1.37 (t, J = 7.6 Hz, 3H). | |
| 120 | 1 | ¹H NMR (400 MHz, CD₃OD) δ 8.13 (d, J = 2.4 Hz, 1H), 7.98 | 365.2 |
| | | (s, 1H), 7.77 (d, J = 8.8 Hz, 1H), 7.73-7.69 (m, 1H), 6.59 (s, | |
| | | 1H), 4.18-4.10 (m, 1H), 3.20-3.15 (m, 1H), 2.81 (t, J = 7.6 | |
| | | Hz, 2H), 2.25-2.23 (m, 2H), 2.17-2.14 (m, 2H), 1.85-1.60 | |
| | | (m, 6H), 1.04 (t, J = 7.6 Hz, 3H). | |
| 121 | 2 | ¹H NMR (400 MHz, CD₃OD) δ 9.49 (s, 1H), 9.26-9.23 (m, | 472.3 |
| | | 1H), 8.40 (s, 1H), 8.09 (s, 1H), 7.79 (s, 1H), 7.74 (d, J = 8.8 | |
| | | Hz, 2H), 7.67 (d, J = 8.8 Hz, 2H), 6.56 (s, 1H), 4.66-4.65 (m, | |
| | | 2H), 4.17-4.12 (m, 1H), 3.24-3.21 (m, 1H), 2.78 (t, J = 7.6 | |
| | | Hz, 2H), 2.33-2.30 (m, 2H), 2.19-2.16 (m, 2H), 1.83-1.55 | |
| | | (m, 6H), 1.03 (t, J = 7.6 Hz, 3H). | |
| 122 | 1 | ¹H NMR (400 MHz, CD₃OD) δ 8.86 (d, J = 1.2 Hz, 1H), 8.42 | 381.3 |
| | | (s, 1H), 7.36 (d, J = 1.2 Hz, 1H), 6.58 (s, 1H), 4.23-4.20 (m, | |
| | | 1H), 4.07 (s, 3H), 3.28-3.23 (m, 1H), 2.79 (t, J = 7.6 Hz, 2H), | |
| | | 2.37-2.35 (m, 2H), 2.21-2.18 (m, 2H), 1.84-1.62 (m, 6H), | |
| | | 1.04 (t, J = 7.6 Hz, 3H). | |
| 123 | 1 | ¹H NMR (400 MHz, CD₃OD δ 8.65 (s, 1H), 8.32 (d, J = 6.8 | 366.3 |
| | | Hz, 1H), 7.46 (d, J = 6.8 Hz, 1H), 6.62 (s, 1H), 4.27-4.21 (m, | |
| | | 1H), 3.30-3.25 (m, 1H), 2.80 (t, J = 7.6 Hz, 3H), 2.36-2.33 | |
| | | (m, 2H), 2.23-2.20 (m, 2H), 1.91-1.69 (m, 6H), 1.04 (t, J = | |
| | | 7.2 Hz, 3H). | |
| 124 | 2 | ¹H NMR (400 MHz, CD₃OD) δ 8.28 (s, 1H), 6.54 (s, 1H), | 400.4 |
| | | 4.16-4.08 (m, 2H), 4.01-3.98 (m, 2H), 3.55-3.49 (m, 2H), | |
| | | 3.27-3.21 (m, 1H), 2.78 (t, J = 7.6 Hz, 2H), 2.33-2.30 (m, | |
| | | 2H), 2.19-2.16 (m, 2H), 1.92-1.80 (m, 2H), 1.78-1.55 (m, | |
| | | 8H), 1.03 (t, J = 7.6 Hz, 3H). | |
| 125 | 1 | ¹H NMR (400 MHz, D₂O) δ 8.67 (d, J = 1.8 Hz, 1H), 8.06 | 434.2 |
| | | (dd, J = 2.4, 8.4 Hz, 1H), 7.95 (s, 1H), 7.74 (d, J = 8.4 Hz, | |
| | | 1H), 6.33 (s, 1H), 4.45 (s, 2H), 3.95-3.80 (m, 1H), 3.70-3.59 | |
| | | (m, 1H), 3.53 (m, 2H), 3.27-3.10 (m, 2H), 2.62 (t, J = 7.6 | |
| | | Hz, 2H), 2.23-1.83 (m, 8H), 1.61 (m, 2H), 1.49-1.25 (m, | |
| | | 4H), 0.86 (t, J = 7.2 Hz, 3H). | |
| 126 | 3 | ¹H NMR (400 MHz, CD₃OD) δ 8.24 (s, 1H), 8.14 (s, 1H), | 498.3 |
| | | 7.62-7.50 (m, 5H), 7.16 (s, 1H), 6.29 (s, 1H), 4.59 (s, 2H), | |
| | | 4.0-3.90 (m, 1H), 3.20-3.16 (m, 1H), 2.92-2.90 (m, 1H), | |
| | | 2.30-2.26 (m, 2H), 2.20-2.00 (m, 4H), 1.93-1.70 (m, 6H), | |
| | | 1.54-1.39 (m, 4H). | |
| 127 | 1 | ¹H NMR (400 MHz, CD₃OD) δ 8.45 (s, 1H), 8.40 (d, J = 9.2 | 365.2 |
| | | Hz, 1H), 8.01 (d, J = 9.2 Hz, 1H), 6.63 (s, 1H), 4.28 (s, 1H), | |
| | | 3.29-3.20 (m, 1H), 2.88-2.76 (m, 5H), 2.41-2.38 (m, 2H), | |
| | | 2.26-2.17 (m, 2H), 1.85-1.69 (m, 6H), 1.09 (t, J = 7.2 Hz, | |
| | | 3H). | |
| 128 | 3 | ¹H NMR (400 MHz, CD₃OD) δ 8.42 (s, 1H), 7.62-7.59 (m, | 392.1 |
| | | 2H), 7.15-7.06 (m, 2H), 6.89 (s, 1H), 3.96-3.93 (m, 1H), | |
| | | 3.86 (s, 1H), 2.87-2.81 (m, 1H), 2.34-2.20 (m, 2H), 2.08- | |
| | | 1.96 (m, 2H), 1.48-1.38 (m, 4H). | |
| 129 | 3 | ¹H NMR (400 MHz, CD₃OD) δ 9.52 (t, J = 1.2 Hz, 1H), 8.41 | 472.2 |
| | | (s, 1H), 8.11 (t, J = 2.0 Hz, 1H), 7.81 (t, J = 2.0 Hz, 1H), 7.76 | |
| | | (dd, J1 = 2.0 Hz, J2 = 8.4 Hz, 2H), 7.69 (d, J = 8.4 Hz, 2H), | |
| | | 6.52 (s, 1H), 4.67 (s, 2H), 4.20-4.14 (m, 1H), 3.29-3.24 (m, | |
| | | 1H), 3.19-3.12 (m, 1H), 2.36-2.33 (m, 2H), 2.22-2.19 (m, | |
| | | 2H), 1.78-1.55 (m, 4H), 1.41 (s, 3H), 1.40 (s, 3H). | |
| 130 | 1 | ¹H NMR (400 MHz, CD₃OD) δ 8.81 (s, 1H), 8.38-8.36 (m, | 422.2 |
| | | 1H), 8.23 (s, 1H), 8.11 (d, J = 8.4 Hz, 1H), 6.63 (s, 1H), 4.77 | |
| | | (s, 2H), 4.22-4.17 (m, 1H), 3.90-3.84 (m, 1H), 3.26-3.20 (m, | |
| | | 1H), 2.83 (t, J = 7.6 Hz, 2H), 2.34-2.32 (m, 2H), 2.19-2.18 | |
| | | (m, 2H), 1.85-1.67 (m, 6H), 1.30 (d, J = 6.0 Hz, 6H), 1.07 (t, | |
| | | J = 7.6 Hz, 3H). | |
| 131 | 1 | ¹H NMR (400 MHz, CD₃OD) δ 8.12 (d, J = 6.8 Hz, 1H), 7.99 | 365.2 |
| | | (s, 1H), 7.00 (d, J = 2.4 Hz, 1H), 6.92 (J₁ = 2.4 Hz, J₂ = 7.2 | |
| | | Hz, 1H), 6.61 (s, 1H), 4.18-4.10 (m, 1H), 3.20-3.15 (m, 1H), | |
| | | 2.81 (t, J = 7.2 Hz, 2H), 2.23-2.20 (m, 2H), 2.20-2.15 (m, | |
| | | 2H), 1.85-1.66 (m, 6H), 1.04 (t, J = 7.6 Hz, 3H). | |
| 132 | 3 | ¹H NMR (400 MHz, D₂O) δ 8.48 (d, J = 5.6 Hz, 2H), 8.07 (s, | 463.4 |
| | | 1H), 6.92 (t, J = 5.6 Hz, 1H), 6.44 (s, 1H), 4.45-4.41 (m, 2H), | |
| | | 4.20-4.13 (m, 1H), 4.09-4.04 (m, 1H), 3.41-3.35 (t, J = 11.6 | |
| | | Hz, 2H), 3.30-3.24 (m, 1H), 2.75-2.69 (q, J = 7.6 Hz, 2H), | |
| | | 2.26-2.23 (m, 2H), 2.15-2.12 (m, 4H), 1.72-1.48 (m, 6H), | |
| | | 1.23 (t, J = 7.6 Hz, 3H). | |
| 133 | 4 | ¹H NMR (400 MHz, DMSO-d₆) δ 11.35 (s, 1H), 9.09 (d, | 500.2 |
| | | J = 8.0 Hz, 1H), 8.85 (s, 1H), 8.33 (s, 1H), 8.23 (d, J = 0.8 Hz, | |
| | | 1H), 7.72 (s, 1H), 7.61 (d, J = 8.4 Hz, 2H), 7.45 (d, J = 8.0 Hz, | |
| | | 2H), 7.10 (s, 1H), 6.19-6.16 (m, 1H), 4.45 (d, J = 5.2 Hz, 2H), | |
| | | 3.79 (s, 1H), 2.63 (t, J = 7.4 Hz, 2H), 2.05-2.03 (m, 2H), 1.82- | |
| | | 1.80 (m,2H), 1.70-1.61 (m, 2H), 1.39-1.18 (m, 10H), 0.88 | |
| | | (t, J = 6.8 Hz, 3H). | |
| 134 | 4 | ¹H NMR (400 MHz, CD₃OD) δ 8.30 (s, 1H), 7.61-7.57 (m, | 424.1 |
| | | 2H), 7.11-7.07 (m, 2H), 6.29 (s, 1H), 4.00-3.95 (m, 1H), | |
| | | 2.87-2.82 (m, 2H), 2.27-2.25 (m, 2H), 2.04-1.99 (m, 2H), | |
| | | 1.80-1.70 (m, 2H), 1.47-1.36 (m, 4H), 1.34 (d, J = 6.8 Hz, | |
| | | 3H), 0.93 (t, J = 7.6 Hz, 3H). | |
| 135 | 3 | ¹H NMR (400 MHz, CD₃OD) δ 8.66 (s, 1H), 7.68-7.64 (m, | 436.1 |
| | | 2H), 7.31 (s, 1H), 7.13 (t, J = 8.8 Hz, 2H), 4.24-4.19 (m, 1H), | |
| | | 3.25-3.22 (m, 1H), 2.35-2.32 (m, 2H), 2.20-2.18 (m, 2H), | |
| | | 1.77-1.62 (m, 4H). | |
| 136 | 3 | ¹H NMR (400 MHz, CD₃OD) δ 8.47 (s, 1H), 7.69-7.65 (m, | 396.2 |
| | | 2H), 7.16-7.12 (m, 2H), 6.59 (s, 1H), 4.21-4.16 (m, 1H), | |
| | | 3.29-3.23 (m, 1H), 2.86 (q, J = 7.6 Hz, 2H), 2.37-2.34 (m, | |
| | | 2H), 2.21-2.18(m, 2H), 1.76-1 62 (m, 4H), 1.39 (t, J = 7.6 | |
| | | Hz, 3H). | |
| 137 | 1 | ¹H NMR (400 MHz, DMSO-d₆) δ 11.31 (s, 1H), 7.88 (s, | 381.2 |
| | | 1H), 6.30 (s, 1H), 5.21 (d, J = 4.8 Hz, 1H), 5.05 (d, J = 8.8 | 383.2 |
| | | Hz, 1H), 4.65-4.60 (m, 1H), 3.84-3.82 (m, 1H), 2.60-2.58 | |
| | | (m, 1H), 1.99 (d, J = 10.4 Hz, 2H), 1.81 (d, J = 11.6 Hz, 2H), | |
| | | 1.73 (q, J = 7.2 Hz, 2H), 1.42-1.24 (m. 6H), 0.89 (t, J = 7.2 | |
| | | Hz, 3H). | |
| 138 | 3 | ¹H NMR (400 MHz, CD₃OD) δ 8.44 (s, 1H), 7.66-7.63 (m, | 422.2 |
| | | 2H), 7.12 (t, J = 8.8 Hz, 2H), 6.57 (s, 1H), 4.21-4.15 (m, 1H), | |
| | | 3.78-3.69 (m, 1H), 3.28-3.24 (m, 1H), 2.46-2.42 (m, 2H), | |
| | | 2.35-2.30 (m, 4H), 2.20-2.13 (m, 3H), 1.99-1.97 (m, 1H), | |
| | | 1.77-1.61 (m, 4H). | |
| 139 | 2 | ¹H NMR (400 MHz, CD₃OD) δ 8.20 (s, 1H), 6.51 (s, 1H), | 330.2 |
| | | 4.11-4.08 (m, 1H), 3.25-3.15 (m, 1H), 2.88 (s, 3H), 2.76 (t, | |
| | | J = 7.2 Hz, 2H), 2.33-2.30 (d, J = 12.4 Hz, 2H), 2.18-2.15 | |
| | | (d, J = 11.2 Hz, 2H), 1.80-1.72 (m, 2H), 1.71- 1.61 (m, 4H), | |
| | | 1.02 (t, J = 7. 2Hz, 3H). | |
| 140 | 2 | ¹H NMR (400 MHz, CD₃OD) δ 8.60 (s, 1H), 6.61 (s, 1H), | 331.2 |
| | | 4.23-4.17 (m, 1H), 3.94 (s, 3H), 3.27-3.24 (m, 1H), 2.79 (t, | |
| | | J = 7.6 Hz, 2H), 2.36-2.33 (m, 2H), 2.21-2.18 (m, 2H), 1.82- | |
| | | 1.67 (m, 6H), 1.02 (t, J = 7.2 Hz, 3H). | |
| 141 | 1 | ¹H NMR (400 MHz, D₂O) δ 8.50 (d, J = 5.2 Hz, 2H), 8.09 (s, | 477.3 |
| | | 1H), 6.93 (t, J = 5.2 Hz, 1H), 6.46 (s, 1H), 4.67-4.43 (m, 2H), | |
| | | 4.20-4.19 (m, 1H), 4.09-4.08 (m, 1H), 3.39 (t, J = 12 Hz, 2H), | |
| | | 3.30-3.29 (m, 1H), 2.70 (t, J = 7.2 Hz, 2H), 2.28-2.25 (m, | |
| | | 2H), 2.17-2.14 (m, 4H), 1.72-1.53 (m, 8H), 0.90 (t, J = 7.6 | |
| | | Hz, 3H). | |
| 142 | 2 | ¹H NMR (400 MHz, CD₃OD) δ 8.17 (s, 1H), 6.53 (s, 1H), | 351.1 |
| | | 4.23-4.13 (m, 1H), 3.24-3.23 (m, 1H), 2.79 (t, J = 7.6 Hz, | 353.0 |
| | | 2H), 2.27-2.23 (m, 2H), 2.20-2.17 (m, 2H), 1.81-1.71 (m, | |
| | | 6H), 1.03 (t, J = 7.4 Hz, 3H). | |
| 143 | 1 | ¹H NMR (400 MHz, CD₃OD) δ 8.53 (s, 1H), 8.22 (s, 1H), | 452.2 |
| | | 7.85 (dd, J₁ = 2.4 Hz, J₂ = 8.4 Hz, 1H), 7.76 (d, J = 8.4 Hz, | |
| | | 1H), 6.52 (s, 1H), 5.00 (q, J = 6.4 Hz, 1H), 4.04-4.03 (m, | |
| | | 1H), 3.74-3.67 (m, 5H), 3.60 (s, 2H), 2.54-2.53 (m, 4H), | |
| | | 2.25 (d, J = 10.8 Hz, 2H), 2.03 (d, J =11.2 Hz, 2H), 1.61 | |
| | | (d, J = 6.4 Hz, 3H), 1.54-1.47 (m, 4H). | |
| 144 | 1 | ¹H NMR (400 MHz, CD₃OD) δ 8.88 (d, J =1.6 Hz, 1H), 8.43 | 436.3 |
| | | (s, 1H), 8.25 (dd, J₁ = 2.0, J₂ = 8.4 Hz, 1H), 8.09 (d, J = 8.4 | |
| | | Hz, 1H), 6.58 (s, 1H), 4.55 (s, 2H), 4.22 (s, 1H), 4.10 (d, J = | |
| | | 11.2 Hz, 2H), 3.86 (t, J = 12 Hz, 2H), 3.75 (s, 1H), 3.49 (d, | |
| | | J = 12.8 Hz, 2H), 3.29 (s, 2H), 2.86 (q, J = 7.6 Hz, 2H), 2.30- | |
| | | 2.27 (m, 2H), 2.08-2.06 (m, 2H), 1.64-1.58 (m, 4H), 1.39 | |
| | | (t, J = 7.6 Hz, 3H). | |
| 145 | 4 | ¹H NMR (400 MHz, CD₃OD) δ 8.36 (s, 1H), 7.58 (q, J = 4.4 | 426.1 |
| | | Hz, 2H), 7.09 (t, J = 8.8 Hz, 2H), 6.52 (s, 1H), 4.53 (q, J = 6.4 | |
| | | Hz, 1H), 4.05-3.95 (m, 1H), 3.30 (s, 3H), 3.06-2.95 (m, 1H), | |
| | | 2.35-2.25 (m, 2H), 2.13-2.03 (m, 2H), 1.60-1.40 (m, 7H). | |
| 146 | 4 | ¹H NMR (400 MHz, CD₃OD) δ 8.36 (s, 1H), 7.60-7.56 (m, | 440.3 |
| | | 2H), 7.10-7.05 (m, 2H), 6.53 (s, 1H), 4.25 (t, H = 7.2Hz, 1H), | |
| | | 4.00-3.97 (m, 1H), 3.29 (s, 3H), 2.89-2.85 (m, 1H), 2.27-2.24 | |
| | | (m, 2H), 2.03-1.79 (m, 4H), 1.52-1.44 (m, 4H), 0.93 (t, J = | |
| | | 7.6 Hz, 3H). | |
| 147 | 4 | ¹H NMR (400 MHz, CD₃OD) δ 8.42 (s, 1H), 7.69-7.56 (m, | 438.2 |
| | | 2H), 7.17-7.06 (m, 2H), 6.55 (s, 1H), 4.15 (br s, 1H), 3.23 | |
| | | (br s, 1H), 2.74-2.63 (m, 1H), 2.33 (br d, J = 13.2 Hz, 2H), | |
| | | 2.17 (br d, J = 11.2 Hz, 2H), 1.95-1.55 (m, 8H), 0.98-0.84 | |
| | | (m, 6H). | |
| 148 | 4 | ¹H NMR (400 MHz, CD₃OD) δ 8.33 (s, 1H), 7.63-7.53 (m, | 412.1 |
| | | 2H), 7.13-7.03 (m, 2H), 6.49 (s, 1H), 4.97 (q, J = 6.8 Hz, | |
| | | 1H), 3.97 (m, 1H), 2.83 (m, 1H), 2.24 m, 2H), 2.01 (m, 2H), | |
| | | 1.58 (d, J = 6,4 Hz, 3H), 1.51-1.35 (m, 4H). | |
| 149 | 4 | ¹H NMR (400 MHz, CD₃OD) δ 8.29 (s, 1H), 7.58 (dd, J = | 438.2 |
| | | 4.8, 8.8 Hz, 2H), 7.08 (t, J = 8.8 Hz, 2H), 6.28 (s, 1H), 3.95 | |
| | | (br s, 1H), 2.96-2.88 (m, 1H), 2.83 (br s, 1H), 2.28-2.18 (m, | |
| | | 2H), 2.06-1.93 (m, 2H), 1.79-1.67 (m, 1H), 1.65-1.54 (m, | |
| | | 1H), 1.49-1.26 (m, 9H), 0.94 (t, J = 7.6 Hz, 3H). | |
| 150 | 4 | ¹H NMR (400 MHz, CD₃OD) δ 8.44 (s, 1H), 7.70-7.59 (m, | 438.2 |
| | | 2H), 7.12 (t, J = 8.8 Hz, 2H), 6.54 (s, 1H), 4.22-4.11 (m, 1H), | |
| | | 3.28-3.19 (m, 1H), 3.08-2.96 (m, 1H), 2.33 (br d, J = 11.6 | |
| | | Hz, 2H), 2.18 (br d, J = 11.2 Hz, 2H), 1.81-1.55 (m, 6H), | |
| | | 1.45-1.28 (m, 5H), 0.96 (t, J = 7.2 Hz, 3H). | |
| 151 | 4 | ¹H NMR (400 MHz, CD₃OD) δ 8.44 (s, 1H), 7.70-7.59 (m, | 424.2 |
| | | 2H), 7.16-7.09 (m, 2H), 6.54 (s, 1H), 4.23-4.10 (m, 1H), | |
| | | 3.28-3.19 (m, 1H), 2.99-2.87 (m, 1H), 2.39-2.29 (m, 2H), | |
| | | 2.23-2.13 (m, 2H), 1.84-1.57 (m, 6H), 1.38 (d, J = 6.8 | |
| | | Hz, 3H), 0.95 (t, J = 7.6 Hz, 3H). | |
| 152 | 4 | ¹H NMR (400 MHz, CD₃OD) δ 8.35 (s, 1H), 7.61-7.55 (m, | 440.2 |
| | | 2H), 7.08 (t, J = 9.0 Hz, 2H), 6.50 (s, 1H), 4.63 (q, J = 6.8 | |
| | | Hz, 1H), 4.05-3.87 (m, 1H), 3.57-3.46 (m, 2H), 2.96-2.76 | |
| | | (m, 1H), 2.34-2.17 (m, 2H), 2.07-1.96 (m, 2H), 1.56 (d, J = | |
| | | 6.6 Hz, 3H), 1.50-1.33 (m, 4H), 1.20 (t, J = 6.8 Hz, 3H). | |
| 153 | 4 | ¹H NMR (400 MHz, CD₃OD) δ 8.35 (s, 1H), 7.61-7.56 (m, | 453.5 |
| | | 2H), 7.08 (t, J = 8.8 Hz, 2H), 6.52 (s, 1H), 4.33 (t, J = 7.2 Hz, | |
| | | 1H), 3.96 (br s, 1H), 3.27 (s, 3H), 2.81 (br s, 1H), 2.30-2.19 | |
| | | (m, 2H), 2.01 (br s, 2H), 1.96-1.87 (m, 1H), 1.85-1.74 (m, | |
| | | 1H), 1.48-1.28 (m, 6H), 0.95 (t, J = 7.6 Hz, 3H). | |
| 154 | 4 | ¹H NMR (400 MHz, DMSO-d₆) δ 11.45 (br s, 1H), 10.04 (s, | 439.5 |
| | | 1H), 8.78 (d, J = 8.0 Hz, 1H), 8.40 (s, 1H), 7.67 (dd, J = 5.2, | |
| | | 9.2 Hz, 2H), 7.16 (t, J = 8.8 Hz, 2H), 6.67 (br d, J = 8.0 Hz, | |
| | | 1H), 6.34 (br s, 1H), 4.62 (t, J = 6.4 Hz, 1H), 3.83 (br s, 1H), | |
| | | 2.95 (br s, 1H), 2.07 (br s,2H), 1.93-1.82 (m, 2H), 1.74 (q, | |
| | | J = 7.2 Hz, 2H), 1.44-1.23 (m, 6H), 0.89 (t, J = 7.6 Hz, 3H). | |
| 155 | 4 | ¹H NMR (400 MHz, CD₃OD) δ 8.33 (s, 1H), 7.60 (dd, J = | 440.2 |
| | | 5.2, 9.2 Hz, 2H), 7.10 (t, J = 8.8 Hz, 2H), 6.38 (s, 1H), | |
| | | 4.02-3.92 (m, 1H), 3.68-3.62 (m, 1H), 3.61-3.55 (m, 1H), | |
| | | 3.41 (s, 3H), 3.25-3.14 (m, 1H), 2.88-2.76 (m, 1H), 2.32- | |
| | | 2.22 (m, 2H), 2.06-1.96 (m, 2H), 1.51-1.41 (m, 4H), 1.38 | |
| | | (d, J = 7 .2 Hz, 3H). | |
| 156 | 4 | ¹H NMR (400 MHz, CD₃OD) δ 8.46 (s, 1H), 7.72-7.61 (m, | 424.2 |
| | | 2H), 7.14 (t, J = 8.4 Hz, 2H), 6.56 (s, 1H), 4.26-4.11 (m, | |
| | | 1H), 3.29-3.20 (m, 1H), 2.95 (q, J = 6.8 Hz, 1H), 2.41-2.30 | |
| | | (m, 2H), 2.26-2.13 (m, 2H), 1.87-1.57 (m, 6H), 1.40 (d, | |
| | | J = 6.8 Hz, 3H), 0.97 (t, J = 7.2 Hz, 3H). | |
| 157 | 4 | ¹H NMR (400 MHz, CD₃OD) 8.60 (d, J = 5.2 Hz, 2H), 8.34 | 492.3 |
| | | (s, 1H), 6.98 (t, J = 5.2 Hz, 1H), 6.52 (s, 1H), 4.65 (d, J = | |
| | | 13.6 Hz, 2H), 4.32-4.23 (m, 1H), 4.13 (br s, 1H), 3.74 (br s, | |
| | | 1H), 3.48-3.38 (m, 2H), 2.92 (m, 1H), 2.26-2.15 (m, 4H), | |
| | | 2.10-2.03 (m, 2H), 1.85-1.70 (m, 4H), 1.62-1.51 (m, 4H), | |
| | | 1.38 (d, J = 7.2 Hz, 3H), 0.95 (t, J = 7.2 Hz, 3H). | |
| 158 | 4 | ¹H NMR (400 MHz, CD₃OD) δ 8.20 (s, 1H), 6.40 (s, 1H), | 496.3 |
| | | 4.07-3.96 (m, 2H), 3.74 (d, J = 12.8 Hz, 2H), 3.61 (br s, 1H), | |
| | | 3.31 (t, J = 5.6 Hz, 4H), 3.06 (t, J = 11.6 Hz, 2H), 2.84-2.75 | |
| | | (m, 1H), 2.15-2.07 (m, 2H), 2.00-1.91 (m, 4 H), 1.89-1.82 | |
| | | (m, 2H), 1.71-1.62 (m, 1H), 1.60-1.54 (m, 2H), 1.44 (t, J = | |
| | | 9.6 Hz, 4H), 1.26 (d, J = 7.2 Hz, 3H), 0.84 (t, J = 7.6 Hz, 3H). | |
| 159 | 1 | ¹H NMR (400 MHz, CD₃OD) δ 8.89 (d, J = 2.0 Hz, 1H), 8.36 | 514.3 |
| | | (br s, 1H), 8.12 (d, J = 2.4 Hz, 1H), 8.10 (d, J = 2.4 Hz, 1H), | |
| | | 6.33 (s, 1H), 4.06 (br s, 1H), 3.78-3.72 (m, 4H), 3.72-3.60 | |
| | | (m, 1H), 3.11-3.03 (m, 4H), 2.90-2.79 (m, 1H), 2.26 (br s, | |
| | | 2H), 2.08-2.01 (m, 2H), 1.82-1.74 (m, 1H), 1.73-1.64 | |
| | | (m, 1H), 1.51 (br t, J = 9.2 Hz, 4H), 1.35 (d, J = 7.2 Hz, 3H), | |
| | | 0.94 (t, J = 7.2 Hz, 3H). | |

### Example 13. IMAP Assay

Activities of compounds against each of TYRO3, AXL, and MERTK as well as against FLT3, were determined using an immobilized metal ion affinity-based fluorescence polarization (“IMAP”) assay. A fluorescently-labeled substrate peptide was phosphorylated in the kinase reaction, after which a binding solution containing large trivalent metal-based nanoparticles was added and the phosphorylated substrate binds to these beads. This reduces the rotational speed of the substrate which is then detected using fluorescence polarization as a read-out.

The substrates used for each enzyme are as follows: AXL: 5F1-KKKKEEIYFFFG-NH₂(SEQ ID NO:1); FLT3: 5F1-KKKKEEIYFFF-NH₂ (SEQ ID NO:2); MERTK: 5F1-EFPIYDFLPAKKK-NH₂ (SEQ ID NO:3); and TYRO3: 5F1-EFPIYDFLPAKKK-NH₂ (SEQ ID NO:4).

This assay was performed in a 384-well, polystyrene microplate in a final volume of 8 μl. Substrate (final concentration=400 nM), ATP (final concentrations=24.7μM for AXL; 2.74 μM for FLT3; 2.69 μM for MERTK; and 54.0 μM for TYRO3), compounds and enzyme (final concentrations=2.5 nM for AXL, FLT3 and TYRO3; 1.5 nM for MERTK) were pipetted in this order into the appropriate reaction buffer (AXL: 50 mM HEPES, pH 8.0, 1 mM DTT, 10 mM MgCl₂, 0.01% BRU35, 0.01% Triton X-100; FLT3: 50 mM HEPES, pH 8.0, 1 mM DTT, 10 mM MgCl₂, 0.01% Triton X-100, 0.01% NP40; MERTK/TYRO3: 50 mM HEPES, pH 8.0, 1 mM DTT, 10 mM MgCl₂, 0.01% Triton X-100) and incubated at room temperature for varying times (AXL, FLT3 and MERTK: 240 minutes; TYRO3: 60 minutes). IMAP binding solution was then dispensed, the plate incubated for 60 minutes at room temperature and the fluorescence polarization measured. Data were analyzed using XLfit.

The results of this assay are set forth in Table 3. In Table 3 “A” indicates an inhibitory constant (Kᵢ) of less than 100 nM; “B” a Kᵢ of between 100 nM and 1 μM; “C” a Kᵢ of greater than 1 μM to 10 μM; and “D” a Kᵢ of greater than 10 μM.

TABLE 3
Activity of Exemplary Compounds of the Invention
against AXL,FLT3, MERTK and TYRO3.

| Compound | AXL | FLT3 | MERTK | TYRO3 |
|---|---|---|---|---|
| 100 | C | A | A | A |
| 101 | D | B | B | B |
| 102 | B | A | A | A |
| 103 | B | A | A | C |
| 104 | C | B | A | A |
| 105 | B | A | A | A |
| 106 | D | B | B | B |
| 107 | C | B | B | B |
| 108 | C | B | A | A |
| 109 | B | A | A | A |
| 110 | C | C | A | A |
| 111 | B | A | A | A |
| 112 | B | A | A | A |
| 113 | C | A | A | A |
| 114 | B | A | A | A |
| 115 | A | A | A | A |
| 116 | B | A | A | A |
| 117 | B | A | A | A |
| 118 | B | A | A | A |
| 119 | B | A | A | A |
| 120 | C | A | A | A |
| 121 | B | A | A | A |
| 122 | B | A | A | A |
| 123 | A | A | A | A |
| 124 | B | B | A | A |
| 125 | B | A | A | A |
| 126 | B | A | A | A |
| 127 | B | A | A | A |
| 128 | C | A | B | B |
| 129 | B | A | A | A |
| 130 | C | A | A | A |
| 131 | C | A | B | A |
| 132 | B | A | A | A |
| 133 | B | B | A | A |
| 134 | C | B | A | A |
| 135 | B | B | A | A |
| 136 | B | A | A | A |
| 137 | C | C | B | B |
| 138 | C | A | A | A |
| 139 | B | A | A | A |
| 140 | C | A | A | A |
| 141 | B | B | A | A |
| 142 | B | B | A | A |
| 143 | C | A | A | A |
| 144 | B | A | A | A |
| 145 | D | C | B | B |
| 146 | D | C | B | A |
| 147 | C | C | B | A |
| 148 | D | B | B | B |
| 149 | C | C | B | A |
| 150 | C | C | A | A |
| 151 | C | B | A | A |
| 152 | D | D | B | B |
| 153 | D | D | C | A |
| 154 | D | D | C | B |
| 155 | D | C | B | A |
| 156 | C | B | A | A |
| 157 | D | D | C | C |
| 158 | C | B | A | A |
| 159 | D | D | C | C |

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, every possible subgroup of the elements is also disclosed, and any element(s) can be removed from the group (i.e., explicitly excluded). It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

One of ordinary skill in the art will recognize, or be able to ascertain using no more than routine experimentation, many equivalents to the specific embodiments of the disclosure described and claimed herein. Such equivalents are intended to be encompassed by the following claims.

1. A compound of structural formula I: 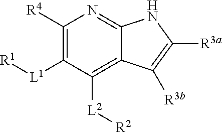
or a pharmaceutically acceptable salt thereof, wherein:
L¹ is a bond, C₁-C₃ alkylene, —CH═CH—, —C≡C—*, —N(R⁵)—C(O)—*, —N(R⁵)—C(O)—CH₂—*, —C(O)—N(R⁵)—*, —C(O)—N(R⁵)—CH₂^{-*}, —O—(C₀-C₂ alkylene)—*, —N(R⁵)—S(O)₂—*, —N(R⁵)—S(O)₂—CH₂—*, —S(O)₂—N(R⁵)—*, —S(O)₂—N(R⁵)—CH₂—*, —N(R⁵)—(C₀-C₂ alkylene)—*, —O—C(O)—*, —O—C(O)—CH₂—*, —C(O)—O—*, or —C(O)—O—CH₂—*, wherein “*” represents a portion of L₁ bound to R¹, and R⁵ is hydrogen, C₁-C₄ alkyl, or C₃-C₇ cycloalkyl, wherein any alkylene, alkyl or cycloalkyl portion of L¹, if present, is optionally substituted;
R¹ is halogen, C₁-C₃ alkyl, aryl, heteroaryl, heterocyclyl or carbocyclyl, wherein R¹ is optionally substituted with up to four different substituents, wherein when L¹ is a bond, C₁ alkylene, —NH—, —C(O)—O—*, or —O—, R¹ is other than C₁ alkyl or C₁ alkyl substituted with halogen; or when L¹ is a bond, R¹ is other than cyclopropyl;
L² is —O—(C₀-C₃ alkylene)-† or —N(R⁶)—(C₀-C₃ alkylene)-†, wherein “†” represents a portion of L² bound to R², R⁶ is hydrogen, C₁-C₄ alkyl, or C₃-C₆ cycloalkyl, wherein any alkylene alkyl or cycloalkyl portion of L², if present, is optionally substituted;
R² is C₁-C₃ alkyl, aryl, heteroaryl, heterocyclyl or carbocyclyl, wherein R² is optionally substituted with up to four different substituents;
R^{3a} is optionally substituted —C₁-C₆ alkyl, —C₂-C₆ alkenyl, —C₂-C₆ alkynyl, —(C₁-C₆ alkylene)—O—(C₁-C₆ alkyl), —(C₀-C₃ alkylene)-aryl, —(C₀-C₃ alkylene)-carbocyclyl, —(C₀-C₃ alkylene)-heterocyclyl, or —(C₀-C₃ alkylene)-heteroaryl;
R^{3b} is hydrogen, halogen, or optionally substituted —C₁-C₄ alkyl; and
R⁴ is hydrogen, halogen, —C₁-C₄ alkyl, or —O—(C₁-C₄ alkyl), wherein any alkyl portion of
R⁴ is optionally substituted.

2. The compound of claim 1, wherein
L¹ is —C(O)—NH—CH₂—*, —C(O)—NH—*, C(O)—O—*, or a bond; or
L¹ is a bond and R¹ is other than cyclopropyl; or,
L¹ is C₁ alkylene, —NH—, —C(O)—O—*, or —O—, and R¹ is other than C₁ alkyl or C₁ alkyl substituted with halogen.

3. The compound of claim 1, wherein R¹ is halogen, —CH₃, or a ring selected from the group consisting of phenyl, cyclohexyl, piperidinyl, azetidinyl, pyridinyl, morpholinyl, pyrimidinyl, pyridazinyl, thiazolyl, tetrahydropyranyl, and pyrazolyl, wherein the ring is optionally substituted.

4. The compound of claim 3, wherein R¹ is halogen, —CH₃, or a ring selected from the group consisting of phenyl, piperidinyl, pyridinyl, pyrimidinyl, pyridazinyl, thiazolyl, tetrahydropyranyl, and pyrazolyl, wherein the ring is optionally substituted.

5. The compound of claim 3, wherein R¹ is a ring substituted with
—R^{1a}-R^{1b}, wherein:
R^{1a} is a bond, —C₁-C₃ alkylene —S(O)₂—, —CH₂—NH—CH₂—, or —C(O)—NH—, and
R^{1b} is hydrogen, —F, —N(C₁-C₄ alkyl)₂, —NH(C₁-C₄ alkyl), —NH₂, —O—(C₁-C₄ alkyl), imidazolyl, morpholinyl, pyrimidinyl, adamantyl, piperidinyl, tetrahydropyranyl, tetrahydrofuranyl, pyrrolidinyl, tetrahydropyrimidinyl, or pyrazolyl, wherein the imidazolyl, morpholinyl, pyrimidinyl, adamantyl, piperidinyl, tetrahydropyranyl, tetrahydrofuranyl, pyrrolidinyl, tetrahydropyrimidinyl, or pyrazolyl is optionally substituted with up to two independent halogen substituents.

6. The compound of claim 5, wherein:
R^{1a} is a bond, —C₁-C₃ alkylene, —S(O)₂—, or —CH₂—NH—CH₂—, and
R^{1b} is hydrogen, —F, —N(C₁-C₄ alkyl)₂, —NH(C₁-C₄ alkyl), —NH₂, —O—(C₁-C₄ alkyl), imidazolyl, morpholinyl, pyrimidinyl, adamantyl, pyrrolidinyl, tetrahydropyrimidinyl, or pyrazolyl, wherein the imidazolyl, morpholinyl, pyrimidinyl, adamantyl, pyrrolidinyl, tetrahydropyrimidinyl, or pyrazolyl is optionally substituted with a single halogen substituent.

7. The compound of claim 3, wherein R¹ is —Br, —CH₃, —CH₂—O—CH(CH₃)₂, —C(O)NHCH₃, 1-(2-dimethylaminoethyl)pyrazol-4-yl, 1-(piperidin-4-yl)pyrazol-4-yl, 1-(pyrimidin-2-yl)piperidin-4-yl, 1-fletrahydrofuran-3-yl)pyrazol-4-yl, 1-(tetrahydrofuran-3-ylmethyl)pyrazol-4-yl, 1-(tetrahydropyran-4-yl)pyrazol-4-yl, 1-(tetrahydropyran-4-ylmethyl)pyrazol-4-yl, 1-(tetrahydropyran-4-ylsulfonyl)azetidin-3-yl, 1-(tetrahydropyran-4-ylsulfonyl)piperidin-4-yl, 1-methylazetidin-3-yl, 1-methylpiperidin-4-yl, 1-methylpyrazol-4-yl, 2-(pyrrolidin-1-yl)pyrimidin-4-yl, 2-aminopyrimidin-4-yl, 4-(1H-imidazol-1-yl)phenyl, 4-(morpholin-4-yl)piperidin-1-yl, 4-(morpholin-4-ylmethyl)cyclohexyl, 4-(morpholin-4-ylmethyl)phenyl, 4-(morpholin-4-ylmethyl)phenyl, 4-aminopyridin-2-yl, 4-fluorophenyl, 5-((((adamant-1-yl)methyl)amino)methyl)pyridin-2-yl, 5-(morpholin-4-ylmethyl)pyridin-2-yl, 5-(morpholin-4-ylsulfonyl)-pyridin-2-yl, 5-aminopyridin-2-yl, 5-fluoropyridin-2-yl, 5-isopropxymethylpyridin-2-yl, 5-(pyrrolidin-1-ylmethyl)pyridin-2-yl, 6-(isopropylamino)pyrimidin-4-yl, 6-(methylamino)pyrimidin-4-yl, 6-aminopyrimidin-4-yl, 6-methoxypyrimidin-4-yl, 6-methylpyridazin-3-yl, morpholin-2-yl, pyridin-2-yl, tetrahydropyran-4-yl, 1-(1,4,5,6-tetrahydropyrimidin-2-yl)piperidin-4-yl, or thiazol-2-yl.

8. The compound of claim 7, wherein R¹ is —CH₃, 1-(2-dimethylaminoethyl)pyrazol-4-yl, 1-(pyrimidin-2-yl)piperidin-4-yl, 1-methylpyrazol-4-yl, 2-(pyrrolidin-1-yl)pyrimidin-4-yl, 2-aminopyrimidin-4-yl, 4-(1H-imidazol-1-yl)phenyl, 4-(morpholin-4-ylmethyl)phenyl, 4-(morpholin-4-ylmethyl)phenyl, 4-aminopyridin-2-yl, 4-fluorophenyl, 5-((((adamant-1-yl)methyl)amino)methyl)pyridin-2-yl, 5-(morpholin-4-ylmethyl)pyridin-2-yl, 5-(morpholin-4-ylsulfonyl)-pyridin-2-yl, 5-aminopyridin-2-yl, 5-fluoropyridin-2-yl, 5-(pyrrolidin-1-ylmethyl)pyridin-2-yl, 6-(isopropylamino)pyrimidin-4-yl, 6-(methylamino)pyrimidin-4-yl, 6-aminopyrimidin-4-yl, 6-methoxypyrimidin-4-yl, 6-methylpyridazin-3-yl, pyridin-2-yl, tetrahydropyran-4-yl, 1-(1,4,5,6-tetrahydropyrimidin-2-yl)piperidin-4-yl, or thiazol-2-yl.

9. The compound of claim 1, wherein L² is —NH— or —O—.

10. The compound of claim 9, wherein L² is —NH—.

11. The compound of claim 1, wherein R² is cyclohexyl, bicyclo[2.2.2]octyl, or bicyclo[1.1.1]pentyl, and wherein R² is optionally substituted.

12. The compound of claim 11, wherein R² is optionally substituted cyclohexyl.

13. The compound of claim 9, wherein R² is 4-aminocyclohexyl, 4-hydroxycyclohexyl, 4-aminobicyclo[2.2.2]octyl, or 4-amino-bicyclo[1.1.1]pentyl.

14. The compound of claim 1, wherein R^{3a} is hydrogen, C₁-C₅ alkyl, C₂-C₅ alkynyl, C₁-C₄ alkylene-O—C₁-C₄ alkyl, C₃-C₆ cycloalkyl, phenyl, benzyl, or an oxygen-containing heterocyclyl, wherein the C₁-C₅ alkyl is optionally substituted with one or more substituents independently selected from halogen, hydroxyl and —OCH₃; and wherein the C₃-C₆ cycloalkyl, phenyl, benzyl, or oxygen-containing containing heterocyclyl is optionally substituted with one or more C₁-C₃ alkyl.

15. The compound of claim 14, wherein R^{3a} is hydrogen, C₁-C₅ alkyl, C₂-C₅ alkynyl, or unsubstituted phenyl, wherein the C₁-C₅ alkyl is optionally substituted with one or more substituents independently selected from halogen and hydroxyl.

16. The compound of claim 14, wherein R^{3a} is hydrogen, methyl, ethyl, n-propyl, isopropyl, n-butyl, sec-butyl, n-pentyl, —CF₃, —C≡CH, hydroxymethyl, methoxymethyl, 1-hydroxyethyl, 2-hydroxyethyl, 1-methoxyethyl, 2-methoxyethyl, 2-fluoroethyl, 1-(fluoromethyl)ethyl, 1-(hydroxymethyl)ethyl, 1-methyl-2-methoxyethyl, trifluoromethyl, 1-hydroxybutyl, 4-hydroxybutyl, 1,4-dihydroxybutyl, 1,4-dimethoxybutyl, 1-(hydroxymethyl)butyl, 1,1,4-trifluorobutyl, n-amyl, sec-amyl, cyclopropyl, 2-ethylcyclopropyl, 2-methylcyclopropyl, cyclobutyl, 3-methylcyclobutyl, cyclopentyl, phenyl, benzyl, oxetan-3-yl, tetrahydrofuran-2-yl, 1-methoxypropyl, pentan-2-yl, pentan-3-yl, 1-hydroxyethyl, 1-ethoxyethyl, 1-methoxybutyl or 1-methoxypropan-2-yl.

17. The compound of claim 16, wherein R^{3a} is hydrogen, methyl, ethyl, n-propyl, n-pentyl, isopropyl, sec-butyl, —C≡CH, cyclobutyl, 1-hydroxyethyl, cyclopentyl, or phenyl.

18. The compound of claim 1, wherein R^{3b} is hydrogen or —Cl.

19. The compound of claim 18, wherein R^{3b} is hydrogen.

20. The compound of claim 1, wherein R⁴ is hydrogen.

21. A pharmaceutical composition comprising a compound of claim 1.

22. A method of treating a cancer associated with overexpression of a TAM kinase or unwanted activity of a TAM kinase in a subject, the method comprising administering, to the subject, the pharmaceutical composition of claim 21.

23. The method of claim 22, wherein the cancer is associated with elevated myeloid infiltration or the cancer is resistant to a checkpoint inhibitor.

24. The method of claim 23, wherein the cancer is a breast cancer, an ovarian cancer, a glioblastoma, a pancreatic ductal adenocarcinoma, a non-small cell lung cancer (NSCLC), a colorectal cancer, a leukemia, a lymphoma, a gastric cancer, a prostate cancer, a pituitary adenoma, a melanoma or a rhabdomyosarcoma.
